(19) United States
(12) Patent Application Publication
(10) Pub. No.: US 2020/0207726 A1
(43) Pub. Date: Jul. 2, 2020

(54) INDOLINYL SULFONAMIDE AND RELATED COMPOUNDS FOR USE AS AGONISTS OF RORY AND THE TREATMENT OF DISEASE

(71) Applicant: Lycera Corporation, Ann Arbor, MI (US)

(72) Inventors: Chad A. VanHuis, Hartland, MI (US); Thomas D. Aicher, Ann Arbor, MI (US); Peter L. Toogood, Ann Arbor, MI (US)

(21) Appl. No.: 16/564,239

(22) Filed: Sep. 9, 2019

Related U.S. Application Data
(63) Continuation of application No. PCT/US2018/021657, filed on Mar. 9, 2018.
(60) Provisional application No. 62/469,584, filed on Mar. 10, 2017.

(51) Int. Cl. C07D 263/56; C07D 209/18; C07D 209/30

(52) U.S. Cl. CPC C07D 263/56; C07D 209/30; C07D 209/18

(57) ABSTRACT

The invention provides aryl indolinyl sulfonamide and related compounds, pharmaceutical compositions, methods of promoting RORγ activity and/or increasing the amount of IL-17 in a subject, and therapeutic uses of the indolinyl sulfonamide and related compounds, such as treating medical conditions in which activation of immune response is beneficial.

### CROSS-REFERENCE TO RELATED APPLICATIONS

This application is a continuation of International Patent Application No. PCT/US2018/021657, filed Mar. 9, 2018, which claims the benefit of and priority to U.S. Provisional Patent Application Ser. No. 62/469,584, filed Mar. 10, 2017; the contents of each of which are hereby incorporated by reference in their entirety.

### FIELD OF THE INVENTION

The invention provides indolinyl sulfonamide and related compounds, pharmaceutical compositions, methods of promoting RORγ activity and/or increasing the amount of IL-17 in a subject, and therapeutic uses of the indolinyl sulfonamide and related compounds, such as treating medical conditions in which activation of immune response is beneficial.

### BACKGROUND

Retinoid-related orphan receptors (ROR) are reported to have an important role in numerous biological processes. See, for example, Dussault et al. in Mech. Dev. (1998) vol. 70, 147-153; and Andre et al. in EMBO J. (1998) vol. 17, 3867-3877. Scientific investigations relating to each of retinoid-related orphan receptors RORα, RORβ, and RORγ have been described in the literature. See, for example, Hirose et al. in Biochem. Biophys. Res. Commun. (1994) vol. 205, 1976-1983; Giguere et al. in Genes. Dev. (1994) vol. 8, 538-553; Medvedev et al. in Gene (1996) vol. 181, 199-206; Ortiz et al. in Mol. Endocrinol. (1995) vol. 9, 1679-1691; and A. M. Jetten in Curr Drug Targets Inflamm Allergy (2004) vol. 3, 395-412). Continuing research in this field is spurred by the promise of developing new therapeutic agents to treat medical disorders associated with retinoid-related orphan receptor activity.

RORγ has been reported to be expressed in high concentration in various tissues, such as thymus, kidney, liver, muscle, and certain fat tissue. See, for example, Hirose et al. in Biochem. Biophys. Res. Commun. (1994) vol. 205, 1976-1983; Medvedev et al. in Gene (1996) vol. 181, 199-206; Ortiz et al. in Mol. Endocrinol. (1995) vol. 9, 1679-1691; and He et al. in Immunity (1998) vol. 9, 797-806. Two isoforms of RORγ have been identified and are referred to as γ1 and γ2 (also referred to as RORγt). See, for example, He et al. in Immunity (1998) vol. 9, 797-806. Expression of the γ2 isoform has been reported to appear in, for example, double-positive thymocytes. See, for example, He et al. in Immunity (1998) vol. 9, 797-806; and Villey et al. in Eur. J. Immunol. (1999) vol. 29, 4072-4080. RORγt plays a critical role in regulating differentiation of Th17 cells, a subset of T helper lymphocytes. See, for example, Ivanov et al. in Cell (2006) vol. 126, 1121-1133. Th17 cells are important for recruiting tumor-killing cytotoxic CD8+ T cells and natural killer cells into the tumor microenvironment. The level of Th17 cells correlated positively with patient survival or slower disease progression in certain cancers. See, for example, Kryczek et al. in Blood (2009) vol 114, 1141-1149; and Sfanos et al. in Clinical Cancer Research (2008) vol 14, 3254-3261. Compounds capable of enhancing RORγt activity are thus contemplated to provide a therapeutic benefit in the treatment of cancer.

Cancer continues to be a significant health problem despite the substantial research efforts and scientific advances reported in the literature for treating this disease. Some of the most frequently diagnosed cancers include prostate cancer, breast cancer, and lung cancer. Prostate cancer is the most common form of cancer in men. Breast cancer remains a leading cause of death in women. Current treatment options for these cancers are not effective for all patients and/or can have substantial adverse side effects.

Accordingly, a need exists for improved treatments for cancer. The present invention addresses this need and provides other related advantages.

### SUMMARY

The invention provides indolinyl sulfonamide and related compounds, pharmaceutical compositions, methods of promoting RORγ activity and/or increasing the amount of IL-17 in a subject, and therapeutic uses of the indolinyl sulfonamide and related compounds, such as treating medical conditions in which activation of immune response is beneficial. In particular, one aspect of the invention provides a collection of aryl indolinyl sulfonamide and related compounds, such as a compound represented by Formula I:

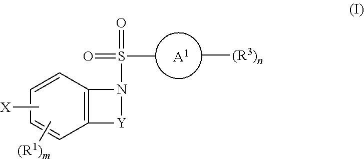

or a pharmaceutically acceptable salt thereof; wherein the variables are as defined in the detailed description. Further description of additional collections of aryl indolinyl sulfonamide and related compounds are described in the detailed description. The compounds may be part of a pharmaceutical composition comprising a pharmaceutically acceptable carrier.

Another aspect of the invention provides a collection of aryl indolyl sulfones and related compounds, such as a compound represented by Formula II:

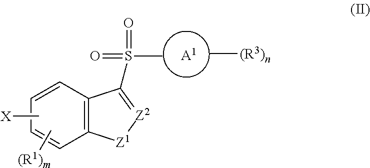

or a pharmaceutically acceptable salt thereof; wherein the variables are as defined in the detailed description. Further description of additional collections of aryl indolyl sulfones and related compounds are described in the detailed description. The compounds may be part of a pharmaceutical composition comprising a pharmaceutically acceptable carrier.

Another aspect of the invention provides a method of treating a subject suffering from a medical disorder. The method comprises administering to the subject a therapeutically effective amount of one or more aryl indolinyl sulfonamide, aryl indolyl sulfone, or related compounds described herein, e.g., a compound of Formula I, I-A, I-A′, I-B, I-B′, I-C, I-D, II, II-A, or II-B, to treat the disorder. A large number of disorders can be treated using the aryl indolinyl sulfonamide, aryl indolyl sulfone, and related compounds described herein. For example, the compounds described herein can be used to treat cancer, a bacterial infection, a fungal infection, or an immune deficiency disorder.

Another aspect of the invention provides a method of promoting the activity of RORγ. The method comprises exposing a RORγ to an effective amount of one or more aryl indolinyl sulfonamide, aryl indolyl sulfone, or related compounds described herein, e.g., a compound of Formula I, I-A, I-A′, I-B, I-B′, I-C, I-D, II, II-A, or II-B, or a pharmaceutical composition described herein.

Another aspect of the invention provides a method of increasing the amount of IL-17 in a subject. The method comprises administering to a subject an effective amount of one or more aryl indolinyl sulfonamide, aryl indolyl sulfone, or related compounds described herein, e.g., a compound of Formula I, I-A, I-A′, I-B, I-B′, I-C, I-D, II, II-A, or II-B, or a pharmaceutical composition described herein, to increase the amount of IL-17 in the subject.

### DETAILED DESCRIPTION

The invention provides indolinyl sulfonamide and related compounds, pharmaceutical compositions, methods of promoting RORγ activity and/or increasing the amount of IL-17 in a subject, and therapeutic uses of the indolinyl sulfonamide and related compounds, such as treating medical conditions in which activation of immune response is beneficial. The practice of the present invention employs, unless otherwise indicated, conventional techniques of organic chemistry, pharmacology, molecular biology (including recombinant techniques), cell biology, biochemistry, and immunology. Such techniques are explained in the literature, such as in “Comprehensive Organic Synthesis” (B. M. Trost & I. Fleming, eds., 1991-1992); “Handbook of experimental immunology” (D. M. Weir & C. C. Blackwell, eds.); “Current protocols in molecular biology” (F. M. Ausubel et al., eds., 1987, and periodic updates); and “Current protocols in immunology” (J. E. Coligan et al., eds., 1991), each of which is herein incorporated by reference in its entirety.

Various aspects of the invention are set forth below in sections; however, aspects of the invention described in one particular section are not to be limited to any particular section. Further, when a variable is not accompanied by a definition, the previous definition of the variable controls.

### Definitions

The terms used herein have their ordinary meaning and the meaning of such terms is independent at each occurrence thereof. That notwithstanding and except where stated otherwise, the following definitions apply throughout the specification and claims. Chemical names, common names, and chemical structures may be used interchangeably to describe the same structure. If a chemical compound is referred to using both a chemical structure and a chemical name, and an ambiguity exists between the structure and the name, the structure predominates. These definitions apply regardless of whether a term is used by itself or in combination with other terms, unless otherwise indicated. Hence, the definition of“alkyl” applies to “alkyl” as well as the “alkyl” portions of “—O-alkyl” etc.

The term “alkyl” refers to a saturated straight or branched hydrocarbon, such as a straight or branched group of 1-12, 1-10, or 1-6 carbon atoms, referred to herein as C₁-C₁₂ alkyl, C₁-C₁₀ alkyl, and C₁-C₆ alkyl, respectively. Exemplary alkyl groups include, but are not limited to, methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, heptyl, octyl, etc.

The term “alkylene” refers to a diradical of an alkyl group. Exemplary alkylene groups include —CH₂—, —CH₂CH₂—, and —CH₂C(H)(CH₃)CH₂—. The term “—(C₀ alkylene)-” refers to a bond. Accordingly, the term “—(C₀₋₃ alkylene)-” encompasses a bond (i.e., C₀) and a —(C₁₋₃ alkylene) group.

The term “heteroalkylene” refers to an alkylene group in which one or more carbon atoms has been replaced by a heteroatom (e.g., N, O, or S). Exemplary heteroalkylene groups include —CH₂O—, —CH₂OCH₂—, and —CH₂CH₂O—. The heteroalkylene group may contain, for example, from 2-4, 2-6, or 2-8 atoms selected from the group consisting of carbon and a heteroatom (e.g., N, O, or S).

The term “cycloalkyl” refers to a monovalent saturated cyclic, bicyclic, or bridged cyclic (e.g., adamantyl) hydrocarbon group of 3-12, 3-8, 4-8, or 4-6 carbons, referred to herein, e.g., as “C₃-C₆ cycloalkyl,” derived from a cycloalkane. Exemplary cycloalkyl groups include cyclohexyl, cyclopentyl, cyclobutyl, and cyclopropyl.

The term “cycloalkylene” refers to a diradical of a cycloalkyl group. Exemplary cycloalkylene groups include

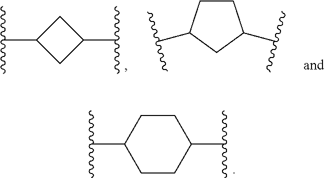

The term “haloalkyl” refers to an alkyl group that is substituted with at least one halogen. Exemplary haloalkyl groups include —CH₂F, —CHF₂, —CF₃, —CH₂CF₃, —CF₂CF₃, and the like.

The term “hydroxyalkyl” refers to an alkyl group that is substituted with at least one hydroxyl. Exemplary hydroxyalkyl groups include —CH₂CH₂OH, —C(H)(OH)CH₃, —CH₂C(H)(OH)CH₂CH₂OH, and the like.

The term “hydroxyhaloalkyl” refers to an alkyl group that is substituted with (i) at least one hydroxyl, and (ii) at least one halogen. Exemplary hydroxyalkyl groups include —C(H)(F)CH₂OH and —C(H)(OH)C(F)H₂, and the like.

The term “aralkyl” refers to an alkyl group substituted with an aryl group. Exemplary aralkyl groups include

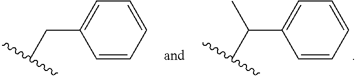

The term “heteroaralkyl” refers to an alkyl group substituted with a heteroaryl group.

The terms “alkenyl” and “alkynyl” are art-recognized and refer to unsaturated aliphatic groups analogous in length and possible substitution to the alkyls described above, but that contain at least one double or triple bond respectively.

The term “cycloalkenyl” refers to a monovalent unsaturated cyclic, bicyclic, or bridged (e.g., adamantyl) carbocyclic hydrocarbon containing at least one C═C double bond. In certain embodiments, the cycloalkenyl contains 5-10, 5-8, or 5-6 carbons, referred to herein, e.g., as “C₅-C₆ cycloalkenyl”. Exemplary cycloalkenyl groups include cyclohexenyl and cyclopentenyl. The term “cycloalkenylene” refers to a diradical of a cycloalkenyl group.

The term “carbocyclylene” refers to a diradical of a carbocyclyl group, wherein a carbocyclyl group is a saturated or unsaturated cyclic, bicyclic, or bridged (e.g., adamantyl) carbocyclic hydrocarbon. In certain embodiments, the carbocyclylene contains 4-10, 5-8, or 5-6 carbons, referred to herein, e.g., as “C₅-C₆ carbocyclylene”.

The term “aryl” is art-recognized and refers to a carbocyclic aromatic group. Representative aryl groups include phenyl, naphthyl, anthracenyl, and the like. Unless specified otherwise, the aromatic ring may be substituted at one or more ring positions with, for example, halogen, azide, alkyl, aralkyl, alkenyl, alkynyl, cycloalkyl, hydroxyl, alkoxyl, amino, nitro, sulfhydryl, imino, amido, carboxylic acid, —C(O)alkyl, —CO₂alkyl, carbonyl, carboxyl, alkylthio, sulfonyl, sulfonamido, sulfonamide, ketone, aldehyde, ester, heterocyclyl, aryl or heteroaryl moieties, —CF₃, —CN, or the like. The term “aryl” also includes polycyclic aromatic ring systems having two or more carbocyclic rings in which two or more carbons are common to two adjoining rings (the rings are “fused rings”) wherein all of the fused rings are aromatic rings, e.g., in a naphthyl group.

The term “arylene” refers to a multivalent radical (e.g., a divalent or trivalent radical) of a carbocyclic aromatic group. The term “phenylene” refers to a multivalent radical (e.g., a divalent or trivalent radical) of benzene. To illustrate, a divalent radical of benzene is illustrated by the formula

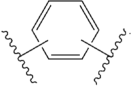

The term “heteroaryl” is art-recognized and refers to aromatic groups that include at least one ring heteroatom. In certain instances, a heteroaryl group contains 1, 2, 3, or 4 ring heteroatoms (e.g., O, N, and S). Representative examples of heteroaryl groups include pyrrolyl, furanyl, thiophenyl, imidazolyl, oxazolyl, thiazolyl, triazolyl, pyrazolyl, pyridinyl, pyrazinyl, pyridazinyl and pyrimidinyl, and the like. Unless specified otherwise, the heteroaryl ring may be substituted at one or more ring positions with, for example, halogen, azide, alkyl, aralkyl, alkenyl, alkynyl, cycloalkyl, hydroxyl, alkoxyl, amino, nitro, sulfhydryl, imino, amido, carboxylic acid, —C(O)alkyl, —CO₂alkyl, carbonyl, carboxyl, alkylthio, sulfonyl, sulfonamido, sulfonamide, ketone, aldehyde, ester, heterocyclyl, aryl or heteroaryl moieties, —CF₃, —CN, or the like. The term “heteroaryl” also includes polycyclic aromatic ring systems having two or more rings in which two or more ring atoms are common to two adjoining rings (the rings are “fused rings”) wherein all of the fused rings are heteroaromatic, e.g., in a naphthyridinyl group. In certain embodiments, the heteroaryl is a 5-6 membered monocylic ring or a 9-10 membered bicyclic ring.

The term “heteroarylene” refers to a multi-valent (e.g., di-valent or trivalent) aromatic group that comprises at least one ring heteroatom. An exemplary “heteroarylene” is pyridinylene, which is a multi-valent radical of pyridine. For example, a divalent radical of pyridine is illustrated by the formula

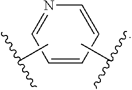

In certain embodiments, the “heteroarylene” is a divalant, 5-6 membered heteroaromatic group containing 1, 2, or 3 ring heteroatoms (e.g., O, N, or S).

The terms ortho, meta, and para are art-recognized and refer to 1,2-, 1,3- and 1,4-disubstituted benzenes, respectively. For example, the names 1,2-dimethylbenzene and ortho-dimethylbenzene are synonymous.

As used herein, the terms “heterocyclic” and “heterocyclyl” represent, for example, an aromatic or nonaromatic ring (e.g., a monocyclic or bicyclic ring) containing one or more heteroatoms. The heteroatoms can be the same or different from each other. Examples of heteratoms include, but are not limited to nitrogen, oxygen and sulfur. Aromatic and nonaromatic heterocyclic rings are well-known in the art. Some nonlimiting examples of aromatic heterocyclic rings include, but are not limited to, pyridine, pyrimidine, indole, purine, quinoline and isoquinoline. Nonlimiting examples of nonaromatic heterocyclic compounds include, but are not limited to, piperidine, piperazine, morpholine, pyrrolidine and pyrazolidine. Examples of oxygen containing heterocyclic rings include, but are not limited to, furan, oxirane, 2H-pyran, 4H-pyran, 2H-chromene, benzofuran, and 2,3-dihydrobenzo[b][1,4]dioxine. Examples of sulfur-containing heterocyclic rings include, but are not limited to, thiophene, benzothiophene, and parathiazine. Examples of nitrogen containing rings include, but are not limited to, pyrrole, pyrrolidine, pyrazole, pyrazolidine, imidazole, imidazoline, imidazolidine, pyridine, piperidine, pyrazine, piperazine, pyrimidine, indole, purine, benzimidazole, quinoline, isoquinoline, triazole, and triazine. Examples of heterocyclic rings containing two different heteroatoms include, but are not limited to, phenothiazine, morpholine, parathiazine, oxazine, oxazole, thiazine, and thiazole. The heterocyclic ring is optionally further substituted at one or more ring positions with, for example, halogen, azide, alkyl, aralkyl, alkenyl, alkynyl, cycloalkyl, hydroxyl, alkoxyl, amino, nitro, sulfhydryl, imino, amido, carboxylic acid, —C(O)alkyl, —CO₂alkyl, carbonyl, carboxyl, alkylthio, sulfonyl, sulfonamido, sulfonamide, ketone, aldehyde, ester, heterocyclyl, aryl or heteroaryl moieties, —CF₃, —CN, or the like. In certain embodiments, the heterocyclyl group is a 3-7 membered ring that, unless specified otherwise, is substituted or unsubstituted.

The term “heterocycloalkyl” refers to a saturated heterocyclyl group having, for example, 3-7 ring atoms selected from carbon and heteroatoms (e.g., O, N, or S).

The term “heterocycloalkylene” refers to a multi-valent (e.g., di-valent or trivalent) saturated heterocyclyl group having, for example, 3-7 ring atoms. An exemplary “heterocycloalkylene” is piperidinylene, which is a multi-valent radical of piperidine. In certain embodiments, the “heterocycloalkylene” is a divalant, 5-6 membered saturated heterocyclyl containing 1 or 2 ring heteroatoms (e.g., O, N, or S).

The phrase “5-6 membered heterocyclic group containing at least one unsaturated carbon atom in the ring” refers to a 5-6 membered heterocyclic group containing at least one ring carbon atom where said carbon atom has a double bond to another atom, such as another atom in the heterocyclic ring or to an exocyclic oxygen atom such that the ring carbon atom is part of a C═O group. Exemplary 5-6 membered heterocyclic groups containing at least one unsaturated carbon atom in the ring include, for example:

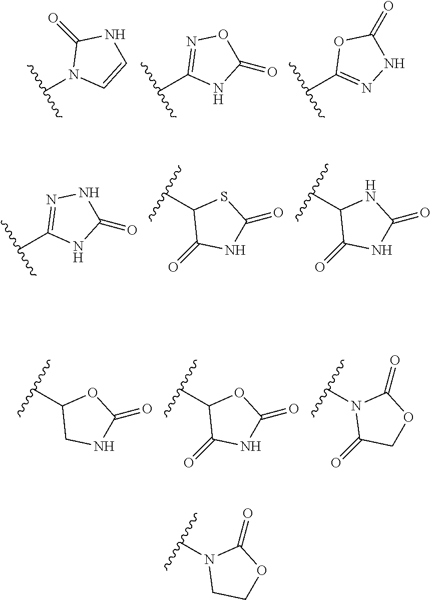

The terms “amine” and “amino” are art-recognized and refer to both unsubstituted and substituted amines, e.g., a moiety that may be represented by the general formulas:

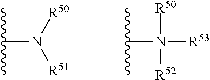

wherein R⁵⁰, R⁵¹, R⁵² and R⁵³ each independently represent a hydrogen, an alkyl, an alkenyl, —(CH₂)ₘ—R⁶¹, or R⁵⁰ and R⁵¹, taken together with the N atom to which they are attached complete a heterocycle having from 4 to 8 atoms in the ring structure; R⁶¹ represents an aryl, a cycloalkyl, a cycloalkenyl, a heterocycle or a polycycle; and m is zero or an integer in the range of 1 to 8. In certain embodiments, only one of R⁵⁰ or R⁵¹ may be a carbonyl, e.g., R⁵⁰, R⁵¹ and the nitrogen together do not form an imide. In other embodiments, R⁵⁰ and R⁵¹ (and optionally R⁵²) each independently represent a hydrogen, an alkyl, an alkenyl, or —(CH₂)ₘ—R⁶¹.

The terms “alkoxyl” or “alkoxy” are art-recognized and refer to an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as may be represented by one of —O-alkyl, —O— alkenyl, —O-alkynyl, and —O—(CH₂)ₘ—R⁶¹, where m and R⁶¹ are described above.

The term “oxo” is art-recognized and refers to a “=0” substituent. For example, a cyclopentane substituted with an oxo group is cyclopentanone.

The symbol “ [symbol] ” indicates a point of attachment.

The term “substituted” means that one or more hydrogens on the atoms of the designated group are replaced with a selection from the indicated group, provided that the atoms' normal valencies under the existing circumstances are not exceeded, and that the substitution results in a stable compound. Combinations of substituents and/or variables are permissible only if such combinations result in stable compounds. The terms “stable compound” or “stable structure” refer to a compound that is sufficiently robust to survive isolation to a useful degree of purity from a reaction mixture, and formulation into an efficacious therapeutic agent.

When any substituent or variable occurs more than one time in any constituent or the compound of the invention, its definition on each occurrence is independent of its definition at every other occurrence, unless otherwise indicated.

It should also be noted that any carbon as well as heteroatom with unsatisfied valences in the text, schemes, examples and tables herein is assumed to have the sufficient number of hydrogen atom(s) to satisfy the valences.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

Certain compounds contained in compositions of the present invention may exist in particular geometric or stereoisomeric forms. Further, certain compounds described herein may be optically active. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. The compounds may contain one or more stereogenic centers. For example, asymmetric carbon atoms may be present in a substituent such as an alkyl group. All such isomers, as well as mixtures thereof, are intended to be included in this invention, such as, for example, racemic mixtures, single enantiomers, diastereomeric mixtures and individual diastereomers. Additional asymmetric centers may be present depending upon the nature of the various substituents on the molecule. Each such asymmetric center will independently produce two optical isomers, and it is intended that all of the possible optical isomers, diastereomers in mixtures, and pure or partially purified compounds are included within the ambit of this invention.

Diastereomeric mixtures can be separated into their individual diastereomers on the basis of their physical chemical differences by methods known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Alternatively, a particular enantiomer of a compound of the present invention may be prepared by asymmetric synthesis. Still further, where the molecule contains a basic functional group (such as amino) or an acidic functional group (such as carboxylic acid) diastereomeric salts are formed with an appropriate optically-active acid or base, followed by resolution of the diastereomers thus formed by fractional crystallization or chromatographic means known in the art, and subsequent recovery of the pure enantiomers.

Individual stereoisomers of the compounds of the invention may, for example, be substantially free of other isomers, or may be admixed, for example, as racemates or with all other, or other selected, stereoisomers. Chiral center(s) in a compound of the present invention can have the S or R configuration as defined by the IUPAC 1974 Recommendations. Further, to the extent a compound described herein may exist as a atropisomer (e.g., substituted biaryls), all forms of such atropisomer are considered part of this invention.

As used herein, the terms “subject” and “patient” are used interchangeably and refer to organisms to be treated by the methods of the present invention. Such organisms preferably include, but are not limited to, mammals (e.g., murines, simians, equines, bovines, porcines, canines, felines, and the like), and most preferably includes humans.

The term “EC₅₀” is art-recognized and refers to the concentration of a compound that is required to achieve 50% of the maximum possible activation of the target.

As used herein, the term “effective amount” refers to the amount of a compound sufficient to effect beneficial or desired results (e.g., a therapeutic, ameliorative, inhibitory or preventative result). An effective amount can be administered in one or more administrations, applications or dosages and is not intended to be limited to a particular formulation or administration route. As used herein, the term “treating” includes any effect, e.g., lessening, reducing, modulating, ameliorating or eliminating, that results in the improvement of the condition, disease, disorder, and the like, or ameliorating a symptom thereof.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see e.g., Martin, Remington's Pharmaceutical Sciences, 15th Ed., Mack Publ. Co., Easton, Pa. [1975].

As used herein, the term “pharmaceutically acceptable salt” refers to any pharmaceutically acceptable salt (e.g., acid or base) of a compound of the present invention which, upon administration to a subject, is capable of providing a compound of this invention or an active metabolite or residue thereof. As is known to those of skill in the art, “salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

Examples of bases include, but are not limited to, alkali metals (e.g., sodium) hydroxides, alkaline earth metals (e.g., magnesium), hydroxides, ammonia, and compounds of formula NW₃, wherein W is C₁₋₄ alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate (also known as toluenesulfonate), undecanoate, and the like. Other examples of salts include anions of the compounds of the present invention compounded with a suitable cation such as Na⁺, NH₄⁺, and NW₄⁺ (wherein W is a C₁₋₄ alkyl group), and the like. Further examples of salts include, but are not limited to: ascorbate, borate, nitrate, phosphate, salicylate, and sulfate. Further, acids which are generally considered suitable for the formation of pharmaceutically useful salts from basic pharmaceutical compounds are discussed, for example, by P. Stahl et al., Camille G. (eds.) Handbook of Pharmaceutical Salts. Properties, Selection and Use. (2002) Zurich: Wiley-VCH; S. Berge et al., Journal of Pharmaceutical Sciences (1977) 66(1) 1-19; P. Gould, International J. of Pharmaceutics (1986) 33 201-217; Anderson et al., The Practice of Medicinal Chemistry (1996), Academic Press, New York; and in The Orange Book (Food & Drug Administration, Washington, D.C. on their website). These disclosures are incorporated herein by reference.

Additional exemplary basic salts include, but are not limited to: ammonium salts, alkali metal salts such as sodium, lithium, and potassium salts, alkaline earth metal salts such as calcium and magnesium salts, salts with organic bases (for example, organic amines) such as dicyclohexylamines, t-butyl amines, and salts with amino acids such as arginine, lysine and the like. Basic nitrogen-containing groups may be quarternized with agents such as lower alkyl halides (e.g., methyl, ethyl, and butyl chlorides, bromides and iodides), dialkyl sulfates (e.g., dimethyl, diethyl, and dibutyl sulfates), long chain halides (e.g., decyl, lauryl, and stearyl chlorides, bromides and iodides), aralkyl halides (e.g., benzyl and phenethyl bromides), and others.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

In addition, when a compound of the invention contains both a basic moiety (such as, but not limited to, a pyridine or imidazole) and an acidic moiety (such as, but not limited to, a carboxylic acid) zwitterions (“inner salts”) may be formed. Such acidic and basic salts used within the scope of the invention are pharmaceutically acceptable (i.e., non-toxic, physiologically acceptable) salts. Such salts of the compounds of the invention may be formed, for example, by reacting a compound of the invention with an amount of acid or base, such as an equivalent amount, in a medium such as one in which the salt precipitates or in an aqueous medium followed by lyophilization.

The present invention includes the compounds of the invention in all their isolated forms (such as any solvates, hydrates, stereoisomers, and tautomers thereof). Further, the invention includes compounds in which one or more of the atoms may be artificially enriched in a particular isotope having the same atomic number, but an atomic mass or mass number different from the atomic mass or mass number predominantly found in nature. The present invention is meant to include all suitable isotopic variations of the compounds of the invention. For example, different isotopic forms of hydrogen (H) include protium (¹H) and deuterium (²H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched compounds can be prepared without undue experimentation by conventional techniques known to those skilled in the art or by processes analogous to those described in the Schemes and Examples herein using appropriate isotopically-enriched reagents and/or intermediates.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

The terms “a” and “an” as used herein mean “one or more” and include the plural unless the context is inappropriate.

The abbreviation “THF” is art-recognized and refers to tetrahydrofuran. The abbreviation “DCM” is art-recognized and refers to dichloromethane. The abbreviation “DMF” is art-recognized and refers to dimethylformamide. The abbreviation “DMA” is art-recognized and refers to dimethylacetamide. The abbreviation “EDTA” is art-recognized and refers to ethylenediaminetetraacetic acid. The abbreviation “TFA” is art-recognized and refers to trifluoroacetic acid. The abbreviation “Ts” is art-recognized and refers to tosylate. The abbreviation “TBS” is art-recognized and refers to tert-butyldimethylsilyl. The abbreviation “DMSO” is art-recognized and refers to dimethylsulfoxide. The abbreviation “Tf” is art-recognized and refers to triflate, or trifluoromethylsulfonate. The abbreviation “Pin” is art-recognized and refers to pinacolato.

As a general matter, compositions specifying a percentage are by weight unless otherwise specified.

### I. Aryl Indolinyl Sulfonamide, Aryl Indolyl Sulfone, and Related Compounds

The invention provides aryl indolinyl sulfonamide, aryl indolyl sulfone, and related compounds. Exemplary compounds are described in the following sections, along with exemplary procedures for making the compounds.

One aspect of the invention provides a compound represented by Formula I:

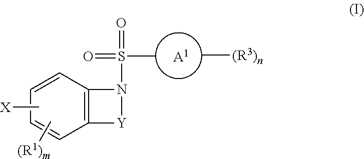

or a pharmaceutically acceptable salt thereof; wherein:

A¹ is phenylene, 5-6 membered heteroarylene, or 3-6 membered heterocycloalkylene; Y is —C(R^{2B})₂—C(R^{2A}) (R^{2B})-ψ, —C(R^{2A})(R^{2B})—C(R^{2B})₂-ψ, —C(R^{2A})═C(R^{2B})-ψ, —C(R^{2B})═C(R^{2A})-ψ, —O—C(R^{2A})(R^{2B})-ψ, —C(R^{2A})═N-ψ, or —N═C(R^{2A})-ψ; wherein ψ is a bond to the sulfonamide ring nitrogen atom in Formula I;

X is phenyl or 5-10 membered heteroaryl, each of which is optionally substituted by 1, 2, 3, or 4 substituents independently selected from the group consisting of C₁₋₆ alkyl, C₃₋₆ cycloalkyl, halogen, C₁₋₆ haloalkyl, hydroxyl, C₁₋₆ alkoxy, C₁₋₆ haloalkoxy, —O—C₃₋₆ cycloalkyl, —O—(C₁₋₆ alkylene)-OH, cyano, —N(R⁴)(R⁵), —S(O)₂—R⁶, acetyl, and C₆₋₁₀ aryl;

R¹ represents independently for each occurrence halogen, C₁₋₆ alkyl, C₁₋₆ haloalkyl, or C₃₋₆ cycloalkyl;

R^{2A} is —(C₁₋₆ alkylene)-A², —(C₃₋₆ cycloalkylene)-A², -(2-6 membered heteroalkylene)-A², —(C₁₋₃ alkylene)-(C₃₋₆ cycloalkylene)-(C₀₋₃ alkylene)-A², —(C₁₋₃ alkylene)-(3-6 membered heterocycloalkylene)-(C₀₋₃ alkylene)-A², —(C₆₋₁₀ arylene)-A², C₂₋₆ hydroxyalkyl, —CO₂R⁴, or hydrogen; wherein:
A² is —CO₂R⁴, —C(O)-A³, —C(O)R⁶, —C(O)N(R⁴)(R⁵), —C(O)N(R⁴)—(C₁₋₄ alkylene)-CO₂R⁴, —C(O)N(R⁴)SO₂R⁴, —C(O)N(R⁴)SO₂A³, —C(O)N(R⁴)—(C₁₋₆ alkylene)-N(R⁷)C(O)R⁶, —C(O)N(R⁴)—(C₁₋₆ alkylene)-SO₂N(R⁷)₂, —C(O)N(R⁴)—(C₁₋₆ alkylene)-CN, —C(O)N(R⁴)—(C₁₋₆ alkylene)-OC(O)R⁶, —N(R⁴)C(O)R⁷, —N(R⁴)C(O)A³, —N(R⁴)C(O)—(C₁₋₆ alkylene)-CO₂R⁴, —N(R⁴)C(O)—(C₁₋₆ alkylene)-N(R⁷)C(O)R⁶, —N(R⁴)C(O)—(C₁₋₆ alkylene)-SO₂N(R⁷)₂, —N(R⁴)C(O)—(C₁₋₆ alkylene)-CN, —N(R⁴)C(O)—(C₁₋₆ alkylene)-OC(O)R⁶, —N(R⁴)C(O)N(R⁴)—(C₁₋₆ alkylene)-CO₂R⁴, —N(R⁴)C(O)N(R⁷)₂, —N(R⁴)CO₂R⁶, —N(R⁴)S(O)₂R⁷, —N(R⁴)S(O)₂N(R⁴)(R⁵), —N(R⁴)(R⁵), hydroxyl, or A³;
A³ is aryl, C₃₋₆ cycloalkyl, or a 5-8 membered heterocyclic group, each of which is optionally substituted with 1, 2, or 3 substituents independently selected from the group consisting of C₁₋₆ alkyl, C₁₋₆ haloalkyl, C₃₋₆ cycloalkyl, halogen, C₁₋₆ alkoxy, C₁₋₆ haloalkoxy, oxo, —C(O)R⁶, —CO₂R⁷, —C(O)N(R⁴)(R⁵), —N(R⁴)C(O)(R⁶), and —N(R⁴)(R⁵); and
any alkylene, cycloalkylene, or heteroalkylene within the definition of R^{2A} is optionally substituted by 1, 2, or 3 substitutents independently selected from the group consisting of hydroxyl and C₁₋₆ alkoxy;

R^{2B} represents independently for each occurrence hydrogen, C₁₋₆ alkyl, or C₁₋₃ haloalkyl;

R³ represents independently for each occurrence hydrogen, C₁₋₆ haloalkyl, halogen, hydroxyl, C₁₋₆ alkyl, C₃₋₆ cycloalkyl, C₁₋₆ alkoxy, C₁₋₆ haloalkoxy, —O—(C₁₋₆ alkylene)-OH, or —O—(C₁₋₆ alkylene)-CO₂R⁴; or two vicinal occurrences of R³ are taken together with intervening atoms to form a 4-6 membered ring optionally substituted with 1, 2, or 3 substituents independently selected from the group consisting of C₁₋₆ alkyl, C₁₋₆ haloalkyl, C₃₋₆ cycloalkyl, halogen, C₁₋₆ alkoxy, C₁₋₆ haloalkoxy, —C(O)R⁶, and —CO₂R⁷;

R⁴ and R⁵ each represent independently for each occurrence hydrogen, C₁₋₆ alkyl, C₁₋₆ haloalkyl, C₁₋₆ hydroxyalkyl, or C₃₋₆ cycloalkyl; or an occurrence of R⁴ and R⁵ attached to the same nitrogen atom are taken together with the nitrogen atom to which they are attached to form a 3-7 membered heterocyclic ring;

R⁶ represents independently for each occurrence C₁₋₆ alkyl, C₃₋₆ cycloalkyl, or aralkyl;

R⁷ represents independently for each occurrence hydrogen, C₁₋₆ alkyl, C₁₋₆ hydroxyalkyl, C₁₋₆ haloalkyl, C₁₋₆ hydroxyhaloalkyl, C₃₋₆ cycloalkyl, C₃₋₆ hydroxycycloalkyl, —(C₁₋₆ alkylene)-(C₃₋₆ cycloalkyl), —(C₁₋₆ alkylene)-(C₂₋₄ alkenyl), or aralkyl;

m is 0, 1, or 2; and

n is 1, 2, or 3.

In certain embodiments, the compound is represented by Formula I.

In certain embodiments, A¹ is phenylene or 5-6 membered heteroarylene. In certain embodiments, A¹ is phenylene. In certain embodiments, A¹ is 5-6 membered heteroarylene. In certain embodiments, -A¹-(R³)ₙ is

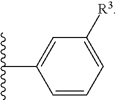

In certain embodiments, Y is —C(R^{2B})₂—C(R^{2A})(R^{2B})-ψ or —C(R^{2A})(R^{2B})—C(R^{2B})₂-ψ; wherein ψ is a bond to the sulfonamide ring nitrogen atom in Formula I. In certain embodiments, Y is —O—C(R^{2A})(R^{2B})-ψ, —C(R^{2A})═N-φ, or —N═C(R^{2A})-ψ; wherein ψ is a bond to the sulfonamide ring nitrogen atom in Formula I. In certain embodiments, Y is —O—C(R^{2A})(R^{2B})-ψ; wherein ψ is a bond to the sulfonamide ring nitrogen atom in Formula I. In certain embodiments, Y is —C(R^{2A})═N-ψ; wherein ψ is a bond to the sulfonamide ring nitrogen atom in Formula I. In certain embodiments, Y is —N═C(R^{2A})-ψ; wherein i is a bond to the sulfonamide ring nitrogen atom in Formula I.

In certain embodiments, Y is —C(R^{2B})₂—C(R^{2A})(R^{2B})-ψ or —C(R^{2A})(R^{2B})—C(R^{2B})₂-ψ; wherein ψ is a bond to the sulfonamide ring nitrogen atom in Formula I; and X is attached at the 6-position of the indolinyl ring.

In certain embodiments, X is phenyl or 5-6 membered heteroaryl, each of which is optionally substituted by 1, 2, or 3 substituents independently selected from the group consisting of C₁₋₆ alkyl, C₃₋₆ cycloalkyl, halogen, C₁₋₆ haloalkyl, hydroxyl, C₁₋₆ alkoxy, C₁₋₆ haloalkoxy, —O—C₃₋₆ cycloalkyl, —O—(C₁₋₆ alkylene)-OH, cyano, —N(R⁴)(R⁵), and C₆₋₁₀ aryl. In certain embodiments, X is phenyl substituted by 1, 2, or 3 substituents independently selected from the group consisting of C₁₋₆ alkyl, C₃₋₆ cycloalkyl, halogen, C₁₋₆ haloalkyl, hydroxyl, C₁₋₆ alkoxy, C₁₋₆ haloalkoxy, —O—C₃₋₆ cycloalkyl, and —O—(C₁₋₆ alkylene)-OH. In certain embodiments, X is phenyl substituted by 1, 2, or 3 substituents independently selected from the group consisting of C₁₋₆ alkyl, C₃₋₆ cycloalkyl, halogen, C₁₋₆ haloalkyl, hydroxyl, C₁₋₆ alkoxy, and C₁₋₆ haloalkoxy. In certain embodiments, X is phenyl substituted by 1, 2, or 3 substituents independently selected from the group consisting of C₁₋₆ alkyl, cyclopropyl, halogen, C₁₋₃ haloalkyl, hydroxyl, C₁₋₃ alkoxy, and C₁₋₃ haloalkoxy. In certain embodiments, X is phenyl substituted by 1, 2, or 3 substituents independently selected from the group consisting of halogen, C₁₋₃ haloalkyl, C₁₋₃ alkoxy, and C₁₋₃ haloalkoxy. In certain embodiments, X is phenyl substituted by 1, 2, or 3 halogens. In certain embodiments, X is phenyl substituted by 1, 2, or 3 substituents independently selected from the group consisting of fluorine and chlorine. In certain embodiments, X is phenyl substituted by 1, 2, or 3 fluorines.

In certain embodiments, X is a 5-6 membered heteroaryl optionally substituted by 1, 2, or 3 substituents independently selected from the group consisting of C₁₋₆ alkyl, C₃₋₆ cycloalkyl, halogen, C₁₋₆ haloalkyl, hydroxyl, C₁₋₆ alkoxy, C₁₋₆ haloalkoxy, —O—C₃₋₆ cycloalkyl, —O—(C₁₋₆ alkylene)-OH, cyano, —N(R⁴)(R⁵), and C₆₋₁₀ aryl. In certain embodiments, X is a 6-membered heteroaryl substituted by 1, 2, or 3 substituents independently selected from the group consisting of C₁₋₆ alkyl, C₃₋₆ cycloalkyl, halogen, C₁₋₆ haloalkyl, hydroxyl, C₁₋₆ alkoxy, and C₁₋₆ haloalkoxy. In certain embodiments, X is a 6-membered heteroaryl substituted by 1, 2, or 3 substituents independently selected from the group consisting of halogen, C₁₋₆ haloalkyl, C₁₋₆ alkoxy, and C₁₋₆ haloalkoxy. In certain embodiments, X is pyridinyl, pyrimidinyl, pyrazolyl, imidazolyl, triazolyl, thiazolyl, oxazolyl, or thiadiazolyl, each of which is optionally substituted by 1, 2, or 3 substituents independently selected from the group consisting of C₁₋₆ alkyl, C₃₋₆ cycloalkyl, halogen, C₁₋₆ haloalkyl, hydroxyl, C₁₋₆ alkoxy, and C₁₋₆ haloalkoxy.

In certain embodiments, R¹ represents independently for each occurrence halogen, C₁₋₆ alkyl, or C₁₋₆ haloalkyl. In certain embodiments, R¹ is halogen. In certain embodiments, R¹ is chloro or fluoro. In certain embodiments, R¹ is halogen, and m is 1.

In certain embodiments, R^{2A} is —(C₁₋₆ alkylene)-A², —(C₃₋₆ cycloalkylene)-A², -(2-6 membered heteroalkylene)-A², —(C₁₋₃ alkylene)-(C₃₋₆ cycloalkylene)-(C₀₋₃ alkylene)-A², —(C₁₋₃ alkylene)-(3-6 membered heterocycloalkylene)-(C₀₋₃ alkylene)-A², or —CO₂R⁴. In certain embodiments, R^{2A} is —(C₁₋₆ alkylene)-A². In certain embodiments, R^{2A} is —(C₂₋₃ alkylene)-A². In certain embodiments, R^{2A} is —(C₁₋₂ alkylene)-A². In certain embodiments, R^{2A} is -(2-6 membered heteroalkylene)-A². In certain embodiments, R^{2A} is —(C₁₋₃ alkylene)-(C₃₋₆ cycloalkylene)-(C₀₋₃ alkylene)-A² or —(C₁₋₃ alkylene)-(3-6 membered heterocycloalkylene)-(C₀₋₃ alkylene)-A². In certain embodiments, R^{2A} is —CO₂R⁴. In certain embodiments, R^{2A} is hydrogen.

In certain embodiments, A² is —CO₂R⁴, —C(O)-A³, —C(O)N(R⁴)(R⁵), —C(O)N(R⁴)—(C₁₋₄ alkylene)-CO₂R⁴, —N(R⁴)C(O)R⁷, —N(R⁴)C(O)-A³, —N(R⁴)C(O)—(C₁₋₆ alkylene)-CO₂R⁴, —N(R⁴)C(O)N(R⁴)—(C₁₋₆ alkylene)-CO₂R⁴, —N(R⁴)CO₂R⁶, or A³. In certain embodiments, A² is —CO₂R⁴. In certain embodiments, A² is —CO₂H. In certain embodiments, A² is —CO₂—(C₁₋₃ alkyl). In certain embodiments, A² is —C(O)-A³.

In certain embodiments, R^{2A} is —(C₁₋₆ alkylene)-A², and A² is —C(O)N(R⁴)—(C₁₋₄ alkylene)-CO₂R⁴, —C(O)N(R⁴)SO₂R⁴, or —C(O)N(R⁴)SO₂A³. In certain embodiments, R^{2A} is —(C₁₋₆ alkylene)-A², and A² is —C(O)N(R⁴)—(C₁₋₆ alkylene)-N(R⁷)C(O)R⁶, —C(O)N(R⁴)—(C₁₋₆ alkylene)-SO₂N(R⁷)₂, —C(O)N(R⁴)—(C₁₋₆ alkylene)-CN, or —C(O)N(R⁴)—(C₁₋₆ alkylene)-OC(O)R⁶. In certain embodiments, R^{2A} is —(C₁₋₆ alkylene)-A², and A² is —N(R⁴)C(O)R⁷, —N(R⁴)C(O)A³, —N(R⁴)C(O)—(C₁₋₆ alkylene)-CO₂R⁴, —N(R⁴)C(O)—(C₁₋₆ alkylene)-N(R⁷)C(O)R⁶, —N(R⁴)C(O)—(C₁₋₆ alkylene)-SO₂N(R⁷)₂, —N(R⁴)C(O)—(C₁₋₆ alkylene)-CN, —N(R⁴)C(O)—(C₁₋₆ alkylene)-OC(O)R⁶, or —N(R⁴)C(O)N(R⁴)—(C₁₋₆ alkylene)-CO₂R⁴. In certain embodiments, R^{2A} is —(C₁₋₆ alkylene)-A², and A² is —N(R⁴)C(O)N(R⁷)₂, —N(R⁴)CO₂R⁶, —N(R⁴)S(O)₂R⁷, or —N(R⁴)S(O)₂N(R⁴)(R⁵). In certain embodiments, R^{2A} is —(C₁₋₆ alkylene)-A², and A² is —N(R⁴)(R⁵) or hydroxyl. In certain embodiments, R^{2A} is —(C₁₋₆ alkylene)-A², and A² is A³.

In certain embodiments, A³ is a 5-8 membered heterocyclic group optionally substituted with 1, 2, or 3 substituents independently selected from the group consisting of C₁₋₆ alkyl, C₁₋₆ haloalkyl, C₃₋₆ cycloalkyl, halogen, C₁₋₆ alkoxy, C₁₋₆ haloalkoxy, oxo, —C(O)R⁶, —CO₂R⁷, and —C(O)N(R⁴)(R⁵). In certain embodiments, A³ is a 5-6 membered heterocyclic group optionally substituted with 1, 2, or 3 substituents independently selected from the group consisting of C₁₋₆ alkyl, C₁₋₆ haloalkyl, halogen, C₁₋₆ alkoxy, oxo, —C(O)R⁶, and —CO₂R⁷. In certain embodiments, A³ is a 5-6 membered saturated heterocyclic group optionally substituted by 1 or 2 substituents independently selected from the group consisting of C₁₋₆ alkyl, C₁₋₆ haloalkyl, C₃₋₆ cycloalkyl, halogen, C₁₋₆ alkoxy, C₁₋₆ haloalkoxy, oxo, —C(O)R⁶, —CO₂R⁷, and —C(O)N(R⁴)(R⁵). In certain embodiments, A³ is a 5-6 membered saturated heterocyclic group optionally substituted by 1 or 2 substituents independently selected from the group consisting of methyl, ethyl, isopropyl, trifluoromethyl, cyclopropyl, C₁₋₆ alkoxy, oxo, and —CO₂R⁷. In certain embodiments, A³ is a 5-6 membered heterocyclic group containing at least one ring carbon atom substituted by oxo and the heterocyclic group being optionally substituted by 1 or 2 substituents independently selected from the group consisting of C₁₋₆ alkyl, C₁₋₆ haloalkyl, C₃₋₆ cycloalkyl, halogen, C₁₋₆ alkoxy, C₁₋₆ haloalkoxy, oxo, —C(O)R⁶, —CO₂R⁷, and —C(O)N(R⁴)(R⁵).

In certain embodiments, R^{2B} is hydrogen or methyl. In certain embodiments, R^{2B} is hydrogen. In certain embodiments, R^{2B} is methyl.

In certain embodiments, R³ represents independently for each occurrence hydrogen, C₁₋₆ haloalkyl, halogen, hydroxyl, C₁₋₆ alkyl, C₃₋₆ cycloalkyl, C₁₋₆ alkoxy, C₁₋₆ haloalkoxy, —O—(C₁₋₆ alkylene)-OH, or —O—(C₁₋₆ alkylene)-CO₂R⁴. In certain embodiments, R³ represents independently for each occurrence C₁₋₆ haloalkyl, halogen, C₁₋₆ alkyl, C₁₋₆ alkoxy, or —O—(C₁₋₆ alkylene)-OH. In certain embodiments, R³ is trifluoromethyl, difluoromethyl, fluoro, chloro, or methoxy. In certain embodiments, R³ is trifluoromethyl.

In certain embodiments, R⁴ and R⁵ each represent independently for each occurrence hydrogen, C₁₋₆ alkyl, or C₃₋₆ cycloalkyl; or an occurrence of R⁴ and R⁵ attached to the same nitrogen atom are taken together with the nitrogen atom to which they are attached to form a 3-7 membered heterocyclic ring. In certain embodiments, R⁴ and R⁵ each represent independently for each occurrence hydrogen or C₁₋₆ alkyl. In certain embodiments, R⁴ is hydrogen. In certain embodiments, R⁵ is hydrogen.

In certain embodiments, R⁶ represents independently for each occurrence C₁₋₆ alkyl or C₃₋₆ cycloalkyl. In certain embodiments, R⁶ is C₁₋₆ alkyl.

In certain embodiments, R⁷ represents independently for each occurrence hydrogen, C₁₋₆ alkyl, C₁₋₆ hydroxyalkyl, C₃₋₆ cycloalkyl, C₃₋₆ hydroxycycloalkyl, or aralkyl. In certain embodiments, R⁷ represents independently for each occurrence hydrogen, C₁₋₆ alkyl, or C₁₋₆ hydroxyalkyl. In certain embodiments, R⁷ is hydrogen. In certain embodiments, R⁷ is C₁₋₆ alkyl.

In certain embodiments, m is 0 or 1. In certain embodiments, m is 1. In certain embodiments, m is 0.

In certain embodiments, n is 1 or 2. In certain embodiments, n is 1.

The definitions of variables in Formula I above encompass multiple chemical groups. The application contemplates embodiments where, for example, i) the definition of a variable is a single chemical group selected from those chemical groups set forth above, ii) the definition of a variable is a collection of two or more of the chemical groups selected from those set forth above, and iii) the compound is defined by a combination of variables in which the variables are defined by (i) or (ii), e.g., such as where A is phenylene and R³ is selected from the group consisting of C₁₋₆ haloalkyl, halogen, hydroxyl, and C₁₋₆ alkyl.

Another aspect of the invention provides a compound represented by Formula I-A or I-A′:

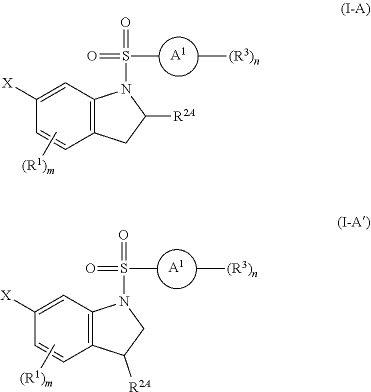

or a pharmaceutically acceptable salt thereof; wherein:

A¹ is phenylene or a 5-6 membered heteroarylene;

X is phenyl or 5-6 membered heteroaryl, each of which is optionally substituted by 1, 2, or 3 substituents independently selected from the group consisting of C₁₋₆ alkyl, C₃₋₆ cycloalkyl, halogen, C₁₋₆ haloalkyl, hydroxyl, C₁₋₆ alkoxy, C₁₋₆ haloalkoxy, —O—C₃₋₆ cycloalkyl, —O—(C₁₋₆ alkylene)-OH, cyano, —N(R⁴)(R⁵), and C₆₋₁₀ aryl;

R¹ represents independently for each occurrence halogen, C₁₋₆ alkyl, or C₁₋₆ haloalkyl;

R^{2A} is —(C₁₋₆ alkylene)-A², —(C₃₋₆ cycloalkylene)-A², -(2-6 membered heteroalkylene)-A², —(C₁₋₃ alkylene)-(C₃₋₆ cycloalkylene)-(C₀₋₃ alkylene)-A², —(C₁₋₃ alkylene)-(3-6 membered heterocycloalkylene)-(C₀₋₃ alkylene)-A², or —CO₂R⁴; wherein A² is —CO₂R⁴, —C(O)-A³, —C(O)N(R⁴)(R⁵), —C(O)N(R⁴)—(C₁₋₄ alkylene)-CO₂R⁴, —N(R⁴)C(O)R⁷, —N(R⁴)C(O)-A³, —N(R⁴)C(O)—(C₁₋₆ alkylene)-CO₂R⁴, —N(R⁴)C(O)N(R⁴)—(C₁₋₆ alkylene)-CO₂R⁴, —N(R⁴)CO₂R⁶, or A³; and A³ is a 5-8 membered heterocyclic group optionally substituted with 1, 2, or 3 substituents independently selected from the group consisting of C₁₋₆ alkyl, C₁₋₆ haloalkyl, C₃₋₆ cycloalkyl, halogen, C₁₋₆ alkoxy, C₁₋₆ haloalkoxy, oxo, —C(O)R⁶, —CO₂R⁷, and —C(O)N(R⁴)(R⁵);

R³ represents independently for each occurrence hydrogen, C₁₋₆ haloalkyl, halogen, hydroxyl, C₁₋₆ alkyl, C₃₋₆ cycloalkyl, C₁₋₆ alkoxy, C₁₋₆ haloalkoxy, —O—(C₁₋₆ alkylene)-OH, or —O—(C₁₋₆ alkylene)-CO₂R⁴;

R⁴ and R⁵ each represent independently for each occurrence hydrogen, C₁₋₆ alkyl, or C₃₋₆ cycloalkyl; or an occurrence of R⁴ and R⁵ attached to the same nitrogen atom are taken together with the nitrogen atom to which they are attached to form a 3-7 membered heterocyclic ring;

R⁶ represents independently for each occurrence C₁₋₆ alkyl, C₃₋₆ cycloalkyl, or aralkyl;

R⁷ represents independently for each occurrence hydrogen, C₁₋₆ alkyl, C₁₋₆ hydroxyalkyl, C₃₋₆ cycloalkyl, C₃₋₆ hydroxycycloalkyl, or aralkyl;

m is 0, 1, or 2; and

n is 1, 2, or 3.

In certain embodiments, the compound is represented by Formula I-A or I-A′.

In certain embodiments, A¹ is phenylene. In certain embodiments, A¹ is 5-6 membered heteroarylene. In certain embodiments, -A¹-(R³)ₙ is

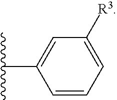

In certain embodiments, X is phenyl substituted by 1, 2, or 3 substituents independently selected from the group consisting of C₁₋₆ alkyl, C₃₋₆ cycloalkyl, halogen, C₁₋₆ haloalkyl, hydroxyl, C₁₋₆ alkoxy, C₁₋₆ haloalkoxy, —O—C₃₋₆ cycloalkyl, and —O—(C₁₋₆ alkylene)-OH. In certain embodiments, X is phenyl substituted by 1, 2, or 3 substituents independently selected from the group consisting of C₁₋₆ alkyl, C₃₋₆ cycloalkyl, halogen, C₁₋₆ haloalkyl, hydroxyl, C₁₋₆ alkoxy, and C₁₋₆ haloalkoxy. In certain embodiments, X is phenyl substituted by 1, 2, or 3 substituents independently selected from the group consisting of C₁₋₆ alkyl, cyclopropyl, halogen, C₁₋₃ haloalkyl, hydroxyl, C₁₋₃ alkoxy, and C₁₋₃ haloalkoxy. In certain embodiments, X is phenyl substituted by 1, 2, or 3 substituents independently selected from the group consisting of halogen, C₁₋₃ haloalkyl, C₁₋₃ alkoxy, and C₁₋₃ haloalkoxy. In certain embodiments, X is phenyl substituted by 1, 2, or 3 halogens. In certain embodiments, X is phenyl substituted by 1, 2, or 3 substituents independently selected from the group consisting of fluorine and chlorine. In certain embodiments, X is phenyl substituted by 1, 2, or 3 fluorines.

In certain embodiments, X is a 5-6 membered heteroaryl optionally substituted by 1, 2, or 3 substituents independently selected from the group consisting of C₁₋₆ alkyl, C₃₋₆ cycloalkyl, halogen, C₁₋₆ haloalkyl, hydroxyl, C₁₋₆ alkoxy, C₁₋₆ haloalkoxy, —O—C₃₋₆ cycloalkyl, —O—(C₁₋₆ alkylene)-OH, cyano, —N(R⁴)(R⁵), and C₆₋₁₀ aryl. In certain embodiments, X is a 6-membered heteroaryl substituted by 1, 2, or 3 substituents independently selected from the group consisting of C₁₋₆ alkyl, C₃₋₆ cycloalkyl, halogen, C₁₋₆ haloalkyl, hydroxyl, C₁₋₆ alkoxy, and C₁₋₆ haloalkoxy. In certain embodiments, X is a 6-membered heteroaryl substituted by 1, 2, or 3 substituents independently selected from the group consisting of halogen, C₁₋₆ haloalkyl, C₁₋₆ alkoxy, and C₁₋₆ haloalkoxy. In certain embodiments, X is pyridinyl, pyrimidinyl, pyrazolyl, imidazolyl, triazolyl, thiazolyl, oxazolyl, or thiadiazolyl, each of which is optionally substituted by 1, 2, or 3 substituents independently selected from the group consisting of C₁₋₆ alkyl, C₃₋₆ cycloalkyl, halogen, C₁₋₆ haloalkyl, hydroxyl, C₁₋₆ alkoxy, and C₁₋₆ haloalkoxy.

In certain embodiments, R¹ is halogen. In certain embodiments, R¹ is chloro or fluoro. In certain embodiments, R¹ is halogen, and m is 1.

In certain embodiments, R^{2A} is —(C₁₋₆ alkylene)-A². In certain embodiments, R^{2A} is —(C₂₋₃ alkylene)-A². In certain embodiments, R^{2A} is —(C₁₋₂ alkylene)-A². In certain embodiments, R^{2A} is -(2-6 membered heteroalkylene)-A². In certain embodiments, R^{2A} is —(C₁₋₃ alkylene)-(C₃₋₆ cycloalkylene)-(C₀₋₃ alkylene)-A² or —(C₁₋₃ alkylene)-(3-6 membered heterocycloalkylene)-(C₀₋₃ alkylene)-A². In certain embodiments, R^{2A} is —CO₂R⁴.

In certain embodiments, A² is —CO₂R⁴. In certain embodiments, A² is —CO₂H. In certain embodiments, A² is —CO₂—(C₁₋₃ alkyl). In certain embodiments, A² is —C(O)-A³.

In certain embodiments, A³ is a 5-6 membered heterocyclic group optionally substituted with 1, 2, or 3 substituents independently selected from the group consisting of C₁₋₆ alkyl, C₁₋₆ haloalkyl, halogen, C₁₋₆ alkoxy, oxo, —C(O)R⁶, and —CO₂R⁷. In certain embodiments, A³ is a 5-6 membered saturated heterocyclic group optionally substituted by 1 or 2 substituents independently selected from the group consisting of C₁₋₆ alkyl, C₁₋₆ haloalkyl, C₃₋₆ cycloalkyl, halogen, C₁₋₆ alkoxy, C₁₋₆ haloalkoxy, oxo, —C(O)R⁶, —CO₂R⁷, and —C(O)N(R⁴)(R⁵). In certain embodiments, A³ is a 5-6 membered saturated heterocyclic group optionally substituted by 1 or 2 substituents independently selected from the group consisting of methyl, ethyl, isopropyl, trifluoromethyl, cyclopropyl, C₁₋₆ alkoxy, oxo, and —CO₂R⁷. In certain embodiments, A³ is a 5-6 membered heterocyclic group containing at least one ring carbon atom substituted by oxo and the heterocyclic group being optionally substituted by 1 or 2 substituents independently selected from the group consisting of C₁₋₆ alkyl, C₁₋₆ haloalkyl, C₃₋₆ cycloalkyl, halogen, C₁₋₆ alkoxy, C₁₋₆ haloalkoxy, oxo, —C(O)R⁶, —CO₂R⁷, and —C(O)N(R⁴)(R⁵).

In certain embodiments, R³ represents independently for each occurrence C₁₋₆ haloalkyl, halogen, C₁₋₆ alkyl, C₁₋₆ alkoxy, or —O—(C₁₋₆ alkylene)-OH. In certain embodiments, R³ is trifluoromethyl, difluoromethyl, fluoro, chloro, or methoxy. In certain embodiments, R³ is trifluoromethyl.

In certain embodiments, R⁴ and R⁵ each represent independently for each occurrence hydrogen or C₁₋₆ alkyl. In certain embodiments, R⁴ is hydrogen. In certain embodiments, R⁵ is hydrogen.

In certain embodiments, R⁶ represents independently for each occurrence C₁₋₆ alkyl or C₃₋₆ cycloalkyl. In certain embodiments, R⁶ is C₁₋₆ alkyl.

In certain embodiments, R⁷ represents independently for each occurrence hydrogen, C₁₋₆ alkyl, or C₁₋₆ hydroxyalkyl. In certain embodiments, R⁷ is hydrogen. In certain embodiments, R⁷ is C₁₋₆ alkyl.

In certain embodiments, m is 0 or 1. In certain embodiments, m is 1. In certain embodiments, m is 0.

In certain embodiments, n is 1 or 2. In certain embodiments, n is 1.

The definitions of variables in Formula I-A and I-A′ above encompass multiple chemical groups. The application contemplates embodiments where, for example, i) the definition of a variable is a single chemical group selected from those chemical groups set forth above, ii) the definition of a variable is a collection of two or more of the chemical groups selected from those set forth above, and iii) the compound is defined by a combination of variables in which the variables are defined by (i) or (ii), e.g., such as where A is phenylene and R³ is selected from the group consisting of C₁₋₆ haloalkyl, halogen, hydroxyl, and C₁₋₆ alkyl.

Another aspect of the invention provides a compound represented by Formula I-B or I-B′:

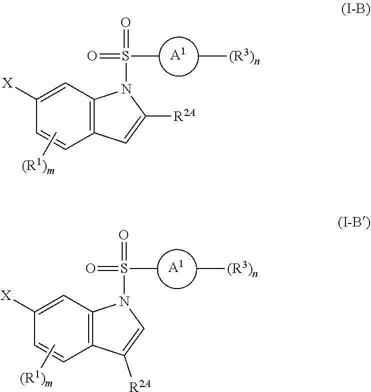

or a pharmaceutically acceptable salt thereof; wherein:

A¹ is phenylene or a 5-6 membered heteroarylene;

X is phenyl or 5-6 membered heteroaryl, each of which is optionally substituted by 1, 2, or 3 substituents independently selected from the group consisting of C₁₋₆ alkyl, C₃₋₆ cycloalkyl, halogen, C₁₋₆ haloalkyl, hydroxyl, C₁₋₆ alkoxy, C₁₋₆ haloalkoxy, —O—C₃₋₆ cycloalkyl, —O—(C₁₋₆ alkylene)-OH, cyano, —N(R⁴)(R⁵), and C₆₋₁₀ aryl;

R¹ represents independently for each occurrence halogen, C₁₋₆ alkyl, or C₁₋₆ haloalkyl;

R^{2A} is —(C₁₋₆ alkylene)-A², —(C₃₋₆ cycloalkylene)-A², -(2-6 membered heteroalkylene)-A², —(C₁₋₃ alkylene)-(C₃₋₆ cycloalkylene)-(C₀₋₃ alkylene)-A², —(C₁₋₃ alkylene)-(3-6 membered heterocycloalkylene)-(C₀₋₃ alkylene)-A², or —CO₂R⁴; wherein A² is —CO₂R⁴, —C(O)-A³, —C(O)N(R⁴)(R⁵), —C(O)N(R⁴)—(C₁₋₄ alkylene)-CO₂R⁴, —N(R⁴)C(O)R⁷, —N(R⁴)C(O)-A³, —N(R⁴)C(O)—(C₁₋₆ alkylene)-CO₂R⁴, —N(R⁴)C(O)N(R⁴)—(C₁₋₆ alkylene)-CO₂R⁴, —N(R⁴)CO₂R⁶, or A³; and A³ is a 5-8 membered heterocyclic group optionally substituted with 1, 2, or 3 substituents independently selected from the group consisting of C₁₋₆ alkyl, C₁₋₆ haloalkyl, C₃₋₆ cycloalkyl, halogen, C₁₋₆ alkoxy, C₁₋₆ haloalkoxy, oxo, —C(O)R⁶, —CO₂R⁷, and —C(O)N(R⁴)(R⁵);

R³ represents independently for each occurrence hydrogen, C₁₋₆ haloalkyl, halogen, hydroxyl, C₁₋₆ alkyl, C₃₋₆ cycloalkyl, C₁₋₆ alkoxy, C₁₋₆ haloalkoxy, —O—(C₁₋₆ alkylene)-OH, or —O—(C₁₋₆ alkylene)-CO₂R⁴;

R⁴ and R⁵ each represent independently for each occurrence hydrogen, C₁₋₆ alkyl, or C₃₋₆ cycloalkyl; or an occurrence of R⁴ and R⁵ attached to the same nitrogen atom are taken together with the nitrogen atom to which they are attached to form a 3-7 membered heterocyclic ring;

R⁶ represents independently for each occurrence C₁₋₆ alkyl, C₃₋₆ cycloalkyl, or aralkyl;

R⁷ represents independently for each occurrence hydrogen, C₁₋₆ alkyl, C₁₋₆ hydroxyalkyl, C₃₋₆ cycloalkyl, C₃₋₆ hydroxycycloalkyl, or aralkyl;

m is 0, 1, or 2; and

n is 1, 2, or 3.

In certain embodiments, the compound is represented by Formula I-B or I-B′.

In certain embodiments, A¹ is phenylene. In certain embodiments, A¹ is 5-6 membered heteroarylene. In certain embodiments, -A¹-(R³)ₙ is

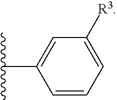

In certain embodiments, X is phenyl substituted by 1, 2, or 3 substituents independently selected from the group consisting of C₁₋₆ alkyl, C₃₋₆ cycloalkyl, halogen, C₁₋₆ haloalkyl, hydroxyl, C₁₋₆ alkoxy, C₁₋₆ haloalkoxy, —O—C₃₋₆ cycloalkyl, and —O—(C₁₋₆ alkylene)-OH. In certain embodiments, X is phenyl substituted by 1, 2, or 3 substituents independently selected from the group consisting of C₁₋₆ alkyl, C₃₋₆ cycloalkyl, halogen, C₁₋₆ haloalkyl, hydroxyl, C₁₋₆ alkoxy, and C₁₋₆ haloalkoxy. In certain embodiments, X is phenyl substituted by 1, 2, or 3 substituents independently selected from the group consisting of C₁₋₆ alkyl, cyclopropyl, halogen, C₁₋₃ haloalkyl, hydroxyl, C₁₋₃ alkoxy, and C₁₋₃ haloalkoxy. In certain embodiments, X is phenyl substituted by 1, 2, or 3 substituents independently selected from the group consisting of halogen, C₁₋₃ haloalkyl, C₁₋₃ alkoxy, and C₁₋₃ haloalkoxy. In certain embodiments, X is phenyl substituted by 1, 2, or 3 halogens. In certain embodiments, X is phenyl substituted by 1, 2, or 3 substituents independently selected from the group consisting of fluorine and chlorine. In certain embodiments, X is phenyl substituted by 1, 2, or 3 fluorines.

In certain embodiments, X is a 5-6 membered heteroaryl optionally substituted by 1, 2, or 3 substituents independently selected from the group consisting of C₁₋₆ alkyl, C₃₋₆ cycloalkyl, halogen, C₁₋₆ haloalkyl, hydroxyl, C₁₋₆ alkoxy, C₁₋₆ haloalkoxy, —O—C₃₋₆ cycloalkyl, —O—(C₁₋₆ alkylene)-OH, cyano, —N(R⁴)(R⁵), and C₆₋₁₀ aryl. In certain embodiments, X is a 6-membered heteroaryl substituted by 1, 2, or 3 substituents independently selected from the group consisting of C₁₋₆ alkyl, C₃₋₆ cycloalkyl, halogen, C₁₋₆ haloalkyl, hydroxyl, C₁₋₆ alkoxy, and C₁₋₆ haloalkoxy. In certain embodiments, X is a 6-membered heteroaryl substituted by 1, 2, or 3 substituents independently selected from the group consisting of halogen, C₁₋₆ haloalkyl, C₁₋₆ alkoxy, and C₁₋₆ haloalkoxy. In certain embodiments, X is pyridinyl, pyrimidinyl, pyrazolyl, imidazolyl, triazolyl, thiazolyl, oxazolyl, or thiadiazolyl, each of which is optionally substituted by 1, 2, or 3 substituents independently selected from the group consisting of C₁₋₆ alkyl, C₃₋₆ cycloalkyl, halogen, C₁₋₆ haloalkyl, hydroxyl, C₁₋₆ alkoxy, and C₁₋₆ haloalkoxy.

In certain embodiments, R¹ is halogen. In certain embodiments, R¹ is chloro or fluoro. In certain embodiments, R¹ is halogen, and m is 1.

In certain embodiments, R^{2A} is —(C₁₋₆ alkylene)-A². In certain embodiments, R^{2A} is —(C₂₋₃ alkylene)-A². In certain embodiments, R^{2A} is —(C₁₋₂ alkylene)-A². In certain embodiments, R^{2A} is -(2-6 membered heteroalkylene)-A². In certain embodiments, R^{2A} is —(C₁₋₃ alkylene)-(C₃₋₆ cycloalkylene)-(C₀₋₃ alkylene)-A² or —(C₁₋₃ alkylene)-(3-6 membered heterocycloalkylene)-(C₀₋₃ alkylene)-A². In certain embodiments, R^{2A} is —CO₂R⁴.

In certain embodiments, A² is —CO₂R⁴. In certain embodiments, A² is —CO₂H. In certain embodiments, A² is —CO₂—(C₁₋₃ alkyl). In certain embodiments, A² is —C(O)-A³.

In certain embodiments, A³ is a 5-6 membered heterocyclic group optionally substituted with 1, 2, or 3 substituents independently selected from the group consisting of C₁₋₆ alkyl, C₁₋₆ haloalkyl, halogen, C₁₋₆ alkoxy, oxo, —C(O)R⁶, and —CO₂R⁷. In certain embodiments, A³ is a 5-6 membered saturated heterocyclic group optionally substituted by 1 or 2 substituents independently selected from the group consisting of C₁₋₆ alkyl, C₁₋₆ haloalkyl, C₃₋₆ cycloalkyl, halogen, C₁₋₆ alkoxy, C₁₋₆ haloalkoxy, oxo, —C(O)R⁶, —CO₂R⁷, and —C(O)N(R⁴)(R⁵). In certain embodiments, A³ is a 5-6 membered saturated heterocyclic group optionally substituted by 1 or 2 substituents independently selected from the group consisting of methyl, ethyl, isopropyl, trifluoromethyl, cyclopropyl, C₁₋₆ alkoxy, oxo, and —CO₂R⁷. In certain embodiments, A³ is a 5-6 membered heterocyclic group containing at least one ring carbon atom substituted by oxo and the heterocyclic group being optionally substituted by 1 or 2 substituents independently selected from the group consisting of C₁₋₆ alkyl, C₁₋₆ haloalkyl, C₃₋₆ cycloalkyl, halogen, C₁₋₆ alkoxy, C₁₋₆ haloalkoxy, oxo, —C(O)R⁶, —CO₂R⁷, and —C(O)N(R⁴)(R⁵).

In certain embodiments, R³ represents independently for each occurrence C₁₋₆ haloalkyl, halogen, C₁₋₆ alkyl, C₁₋₆ alkoxy, or —O—(C₁₋₆ alkylene)-OH. In certain embodiments, R³ is trifluoromethyl, difluoromethyl, fluoro, chloro, or methoxy. In certain embodiments, R³ is trifluoromethyl.

In certain embodiments, R⁴ and R⁵ each represent independently for each occurrence hydrogen or C₁₋₆ alkyl. In certain embodiments, R⁴ is hydrogen. In certain embodiments, R⁵ is hydrogen.

In certain embodiments, R⁶ represents independently for each occurrence C₁₋₆ alkyl or C₃₋₆ cycloalkyl. In certain embodiments, R⁶ is C₁₋₆ alkyl.

In certain embodiments, R⁷ represents independently for each occurrence hydrogen, C₁₋₆ alkyl, or C₁₋₆ hydroxyalkyl. In certain embodiments, R⁷ is hydrogen. In certain embodiments, R⁷ is C₁₋₆ alkyl.

In certain embodiments, m is 0 or 1. In certain embodiments, m is 1. In certain embodiments, m is 0.

In certain embodiments, n is 1 or 2. In certain embodiments, n is 1.

The definitions of variables in Formula I-B and I-B′ above encompass multiple chemical groups. The application contemplates embodiments where, for example, i) the definition of a variable is a single chemical group selected from those chemical groups set forth above, ii) the definition of a variable is a collection of two or more of the chemical groups selected from those set forth above, and iii) the compound is defined by a combination of variables in which the variables are defined by (i) or (ii), e.g., such as where A is phenylene and R³ is selected from the group consisting of C₁₋₆ haloalkyl, halogen, hydroxyl, and C₁₋₆ alkyl.

Another aspect of the invention provides a compound represented by Formula I-C:

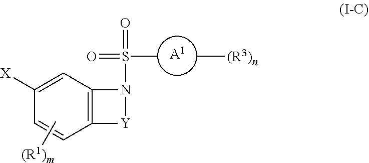

or a pharmaceutically acceptable salt thereof; wherein:

A¹ is phenylene or a 5-6 membered heteroarylene;

Y is —O—C(R^{2A})(R^{2B})-ψ, —C(R^{2A})═N-ψ, or —N═C(R^{2A})-ψ; wherein ψ is a bond to the sulfonamide ring nitrogen atom in Formula I-C;

X is phenyl or 5-6 membered heteroaryl, each of which is optionally substituted by 1, 2, or 3 substituents independently selected from the group consisting of C₁₋₆ alkyl, C₃₋₆ cycloalkyl, halogen, C₁₋₆ haloalkyl, hydroxyl, C₁₋₆ alkoxy, C₁₋₆ haloalkoxy, —O—C₃₋₆ cycloalkyl, —O—(C₁₋₆ alkylene)-OH, cyano, —N(R⁴)(R⁵), and C₆₋₁₀ aryl;

R¹ represents independently for each occurrence halogen, C₁₋₆ alkyl, or C₁₋₆ haloalkyl;

R^{2A} is —(C₁₋₆ alkylene)-A², —(C₃₋₆ cycloalkylene)-A², -(2-6 membered heteroalkylene)-A², —(C₁₋₃ alkylene)-(C₃₋₆ cycloalkylene)-(C₀₋₃ alkylene)-A², —(C₁₋₃ alkylene)-(3-6 membered heterocycloalkylene)-(C₀₋₃ alkylene)-A², or —CO₂R⁴; wherein A² is —CO₂R⁴, —C(O)-A³, —C(O)N(R⁴)(R⁵), —C(O)N(R⁴)—(C₁₋₄ alkylene)-CO₂R⁴, —N(R⁴)C(O)R⁷, —N(R⁴)C(O)-A³, —N(R⁴)C(O)—(C₁₋₆ alkylene)-CO₂R⁴, —N(R⁴)C(O)N(R⁴)—(C₁₋₆ alkylene)-CO₂R⁴, —N(R⁴)CO₂R⁶, or A³; and A³ is a 5-8 membered heterocyclic group optionally substituted with 1, 2, or 3 substituents independently selected from the group consisting of C₁₋₆ alkyl, C₁₋₆ haloalkyl, C₃₋₆ cycloalkyl, halogen, C₁₋₆ alkoxy, C₁₋₆ haloalkoxy, oxo, —C(O)R⁶, —CO₂R⁷, and —C(O)N(R⁴)(R⁵);

R^{2B} is hydrogen, C₁₋₆ alkyl, or C₁₋₃ haloalkyl;

R³ represents independently for each occurrence hydrogen, C₁₋₆ haloalkyl, halogen, hydroxyl, C₁₋₆ alkyl, C₃₋₆ cycloalkyl, C₁₋₆ alkoxy, C₁₋₆ haloalkoxy, —O—(C₁₋₆ alkylene)-OH, or —O—(C₁₋₆ alkylene)-CO₂R⁴;

R⁴ and R⁵ each represent independently for each occurrence hydrogen, C₁₋₆ alkyl, or C₃₋₆ cycloalkyl; or an occurrence of R⁴ and R⁵ attached to the same nitrogen atom are taken together with the nitrogen atom to which they are attached to form a 3-7 membered heterocyclic ring;

R⁶ represents independently for each occurrence C₁₋₆ alkyl, C₃₋₆ cycloalkyl, or aralkyl;

R⁷ represents independently for each occurrence hydrogen, C₁₋₆ alkyl, C₁₋₆ hydroxyalkyl, C₃₋₆ cycloalkyl, C₃₋₆ hydroxycycloalkyl, or aralkyl;

m is 0, 1, or 2; and

n is 1, 2, or 3.

In certain embodiments, the compound is represented by Formula I-C.

In certain embodiments, A¹ is phenylene. In certain embodiments, A¹ is 5-6 membered heteroarylene. In certain embodiments, -A¹-(R³)ₙ is

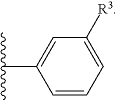

In certain embodiments, Y is —O—C(R^{2A})(R^{2B})-ψ; wherein ψ is a bond to the sulfonamide ring nitrogen atom in Formula I-C. In certain embodiments, Y is-C(R^{2A})═N-ψ; wherein ψ is a bond to the sulfonamide ring nitrogen atom in Formula I-C. In certain embodiments, Y is —N═C(R^{2A})-ψ; wherein ψ is a bond to the sulfonamide ring nitrogen atom in Formula I-C.

In certain embodiments, X is phenyl substituted by 1, 2, or 3 substituents independently selected from the group consisting of C₁₋₆ alkyl, C₃₋₆ cycloalkyl, halogen, C₁₋₆ haloalkyl, hydroxyl, C₁₋₆ alkoxy, C₁₋₆ haloalkoxy, —O—C₃₋₆ cycloalkyl, and —O—(C₁₋₆ alkylene)-OH. In certain embodiments, X is phenyl substituted by 1, 2, or 3 substituents independently selected from the group consisting of C₁₋₆ alkyl, C₃₋₆ cycloalkyl, halogen, C₁₋₆ haloalkyl, hydroxyl, C₁₋₆ alkoxy, and C₁₋₆ haloalkoxy. In certain embodiments, X is phenyl substituted by 1, 2, or 3 substituents independently selected from the group consisting of C₁₋₆ alkyl, cyclopropyl, halogen, C₁₋₃ haloalkyl, hydroxyl, C₁₋₃ alkoxy, and C₁₋₃ haloalkoxy. In certain embodiments, X is phenyl substituted by 1, 2, or 3 substituents independently selected from the group consisting of halogen, C₁₋₃ haloalkyl, C₁₋₃ alkoxy, and C₁₋₃ haloalkoxy. In certain embodiments, X is phenyl substituted by 1, 2, or 3 halogens. In certain embodiments, X is phenyl substituted by 1, 2, or 3 substituents independently selected from the group consisting of fluorine and chlorine. In certain embodiments, X is phenyl substituted by 1, 2, or 3 fluorines.

In certain embodiments, X is a 5-6 membered heteroaryl optionally substituted by 1, 2, or 3 substituents independently selected from the group consisting of C₁₋₆ alkyl, C₃₋₆ cycloalkyl, halogen, C₁₋₆ haloalkyl, hydroxyl, C₁₋₆ alkoxy, C₁₋₆ haloalkoxy, —O—C₃₋₆ cycloalkyl, —O—(C₁₋₆ alkylene)-OH, cyano, —N(R⁴)(R⁵), and C₆₋₁₀ aryl. In certain embodiments, X is a 6-membered heteroaryl substituted by 1, 2, or 3 substituents independently selected from the group consisting of C₁₋₆ alkyl, C₃₋₆ cycloalkyl, halogen, C₁₋₆ haloalkyl, hydroxyl, C₁₋₆ alkoxy, and C₁₋₆ haloalkoxy. In certain embodiments, X is a 6-membered heteroaryl substituted by 1, 2, or 3 substituents independently selected from the group consisting of halogen, C₁₋₆ haloalkyl, C₁₋₆ alkoxy, and C₁₋₆ haloalkoxy. In certain embodiments, X is pyridinyl, pyrimidinyl, pyrazolyl, imidazolyl, triazolyl, thiazolyl, oxazolyl, or thiadiazolyl, each of which is optionally substituted by 1, 2, or 3 substituents independently selected from the group consisting of C₁₋₆ alkyl, C₃₋₆ cycloalkyl, halogen, C₁₋₆ haloalkyl, hydroxyl, C₁₋₆ alkoxy, and C₁₋₆ haloalkoxy.

In certain embodiments, R¹ is halogen. In certain embodiments, R¹ is chloro or fluoro. In certain embodiments, R¹ is halogen, and m is 1.

In certain embodiments, R^{2A} is —(C₁₋₆ alkylene)-A². In certain embodiments, R^{2A} is —(C₂₋₃ alkylene)-A². In certain embodiments, R^{2A} is —(C₁₋₂ alkylene)-A². In certain embodiments, R^{2A} is -(2-6 membered heteroalkylene)-A². In certain embodiments, R^{2A} is —(C₁₋₃ alkylene)-(C₃₋₆ cycloalkylene)-(C₀₋₃ alkylene)-A² or —(C₁₋₃ alkylene)-(3-6 membered heterocycloalkylene)-(C₀₋₃ alkylene)-A². In certain embodiments, R^{2A} is —CO₂R⁴.

In certain embodiments, A² is —CO₂R⁴. In certain embodiments, A² is —CO₂H. In certain embodiments, A² is —CO₂—(C₁₋₃ alkyl). In certain embodiments, A² is —C(O)-A³.

In certain embodiments, A³ is a 5-6 membered heterocyclic group optionally substituted with 1, 2, or 3 substituents independently selected from the group consisting of C₁₋₆ alkyl, C₁₋₆ haloalkyl, halogen, C₁₋₆ alkoxy, oxo, —C(O)R⁶, and —CO₂R⁷. In certain embodiments, A³ is a 5-6 membered saturated heterocyclic group optionally substituted by 1 or 2 substituents independently selected from the group consisting of C₁₋₆ alkyl, C₁₋₆ haloalkyl, C₃₋₆ cycloalkyl, halogen, C₁₋₆ alkoxy, C₁₋₆ haloalkoxy, oxo, —C(O)R⁶, —CO₂R⁷, and —C(O)N(R⁴)(R⁵). In certain embodiments, A³ is a 5-6 membered saturated heterocyclic group optionally substituted by 1 or 2 substituents independently selected from the group consisting of methyl, ethyl, isopropyl, trifluoromethyl, cyclopropyl, C₁₋₆ alkoxy, oxo, and —CO₂R⁷. In certain embodiments, A³ is a 5-6 membered heterocyclic group containing at least one ring carbon atom substituted by oxo and the heterocyclic group being optionally substituted by 1 or 2 substituents independently selected from the group consisting of C₁₋₆ alkyl, C₁₋₆ haloalkyl, C₃₋₆ cycloalkyl, halogen, C₁₋₆ alkoxy, C₁₋₆ haloalkoxy, oxo, —C(O)R⁶, —CO₂R⁷, and —C(O)N(R⁴)(R⁵).

In certain embodiments, R^{2B} is hydrogen or methyl. In certain embodiments, R^{2B} is hydrogen. In certain embodiments, R^{2B} is methyl.

In certain embodiments, R³ represents independently for each occurrence C₁₋₆ haloalkyl, halogen, C₁₋₆ alkyl, C₁₋₆ alkoxy, or —O—(C₁₋₆ alkylene)-OH. In certain embodiments, R³ is trifluoromethyl, difluoromethyl, fluoro, chloro, or methoxy. In certain embodiments, R³ is trifluoromethyl.

In certain embodiments, R⁴ and R⁵ each represent independently for each occurrence hydrogen or C₁₋₆ alkyl. In certain embodiments, R⁴ is hydrogen. In certain embodiments, R⁵ is hydrogen.

In certain embodiments, R⁶ represents independently for each occurrence C₁₋₆ alkyl or C₃₋₆ cycloalkyl. In certain embodiments, R⁶ is C₁₋₆ alkyl.

In certain embodiments, R⁷ represents independently for each occurrence hydrogen, C₁₋₆ alkyl, or C₁₋₆ hydroxyalkyl. In certain embodiments, R⁷ is hydrogen. In certain embodiments, R⁷ is C₁₋₆ alkyl.

In certain embodiments, m is 0 or 1. In certain embodiments, m is 1. In certain embodiments, m is 0.

In certain embodiments, n is 1 or 2. In certain embodiments, n is 1.

The definitions of variables in Formula I-C above encompass multiple chemical groups. The application contemplates embodiments where, for example, i) the definition of a variable is a single chemical group selected from those chemical groups set forth above, ii) the definition of a variable is a collection of two or more of the chemical groups selected from those set forth above, and iii) the compound is defined by a combination of variables in which the variables are defined by (i) or (ii), e.g., such as where A is phenylene and R³ is selected from the group consisting of C₁₋₆ haloalkyl, halogen, hydroxyl, and C₁₋₆ alkyl.

Another aspect of the invention provides a compound represented by Formula I-D:

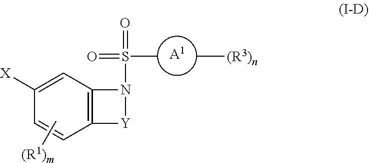

or a pharmaceutically acceptable salt thereof; wherein:

A¹ is phenylene or a 5-6 membered heteroarylene;

Y is —O—C(R^{2A})(R^{2B})-ψ; wherein ψ is a bond to the sulfonamide ring nitrogen atom in Formula I-D;

X is:
(i) phenyl substituted by 1, 2, or 3 substituents independently selected from the group consisting of C₁₋₆ alkyl, C₃₋₆ cycloalkyl, halogen, C₁₋₆ haloalkyl, hydroxyl, C₁₋₆ alkoxy, C₁₋₆ haloalkoxy, —O—C₃₋₆ cycloalkyl, —O—(C₁₋₆ alkylene)-OH, cyano, —N(R⁴)(R⁵), and C₆₋₁₀ aryl; or
(ii) 5-6 membered heteroaryl optionally substituted by 1, 2, or 3 substituents independently selected from the group consisting of C₁₋₆ alkyl, C₃₋₆ cycloalkyl, halogen, C₁₋₆ haloalkyl, hydroxyl, C₁₋₆ alkoxy, C₁₋₆ haloalkoxy, —O—C₃₋₆ cycloalkyl, —O—(C₁₋₆ alkylene)-OH, cyano, —N(R⁴)(R⁵), and C₆₋₁₀ aryl;

R¹ represents independently for each occurrence halogen, C₁₋₆ alkyl, or C₁₋₆ haloalkyl;

R^{2A} is hydrogen;

R^{2B} is hydrogen, C₁₋₆ alkyl, or C₁₋₃ haloalkyl;

R³ represents independently for each occurrence C₁₋₆ haloalkyl, halogen, hydroxyl, C₁₋₆ alkyl, C₃₋₆ cycloalkyl, C₁₋₆ alkoxy, C₁₋₆ haloalkoxy, —O—(C₁₋₆ alkylene)-OH, or —O—(C₁₋₆ alkylene)-CO₂R⁴;

R⁴ and R⁵ each represent independently for each occurrence hydrogen, C₁₋₆ alkyl, or C₃₋₆ cycloalkyl; or an occurrence of R⁴ and R⁵ attached to the same nitrogen atom are taken together with the nitrogen atom to which they are attached to form a 3-7 membered heterocyclic ring;

m is 0, 1, or 2; and

n is 1, 2, or 3.

In certain embodiments, the compound is represented by Formula I-D.

In certain embodiments, A¹ is phenylene. In certain embodiments, A¹ is 5-6 membered heteroarylene. In certain embodiments, -A¹-(R³)ₙ is

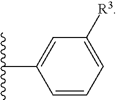

In certain embodiments, X is phenyl substituted by 1, 2, or 3 substituents independently selected from the group consisting of C₁₋₆ alkyl, C₃₋₆ cycloalkyl, halogen, C₁₋₆ haloalkyl, hydroxyl, C₁₋₆ alkoxy, C₁₋₆ haloalkoxy, —O—C₃₋₆ cycloalkyl, and —O—(C₁₋₆ alkylene)-OH. In certain embodiments, X is phenyl substituted by 1, 2, or 3 substituents independently selected from the group consisting of C₁₋₆ alkyl, C₃₋₆ cycloalkyl, halogen, C₁₋₆ haloalkyl, hydroxyl, C₁₋₆ alkoxy, and C₁₋₆ haloalkoxy. In certain embodiments, X is phenyl substituted by 1, 2, or 3 substituents independently selected from the group consisting of C₁₋₆ alkyl, cyclopropyl, halogen, C₁₋₃ haloalkyl, hydroxyl, C₁₋₃ alkoxy, and C₁₋₃ haloalkoxy. In certain embodiments, X is phenyl substituted by 1, 2, or 3 substituents independently selected from the group consisting of halogen, C₁₋₃ haloalkyl, C₁₋₃ alkoxy, and C₁₋₃ haloalkoxy. In certain embodiments, X is phenyl substituted by 1, 2, or 3 halogens. In certain embodiments, X is phenyl substituted by 1, 2, or 3 substituents independently selected from the group consisting of fluorine and chlorine. In certain embodiments, X is phenyl substituted by 1, 2, or 3 fluorines.

In certain embodiments, X is a 5-6 membered heteroaryl optionally substituted by 1, 2, or 3 substituents independently selected from the group consisting of C₁₋₆ alkyl, C₃₋₆ cycloalkyl, halogen, C₁₋₆ haloalkyl, hydroxyl, C₁₋₆ alkoxy, C₁₋₆ haloalkoxy, —O—C₃₋₆ cycloalkyl, —O—(C₁₋₆ alkylene)-OH, cyano, —N(R⁴)(R⁵), and C₆₋₁₀ aryl. In certain embodiments, X is a 6-membered heteroaryl substituted by 1, 2, or 3 substituents independently selected from the group consisting of C₁₋₆ alkyl, C₃₋₆ cycloalkyl, halogen, C₁₋₆ haloalkyl, hydroxyl, C₁₋₆ alkoxy, and C₁₋₆ haloalkoxy. In certain embodiments, X is a 6-membered heteroaryl substituted by 1, 2, or 3 substituents independently selected from the group consisting of halogen, C₁₋₆ haloalkyl, C₁₋₆ alkoxy, and C₁₋₆ haloalkoxy. In certain embodiments, X is pyridinyl, pyrimidinyl, pyrazolyl, imidazolyl, triazolyl, thiazolyl, oxazolyl, or thiadiazolyl, each of which is optionally substituted by 1, 2, or 3 substituents independently selected from the group consisting of C₁₋₆ alkyl, C₃₋₆ cycloalkyl, halogen, C₁₋₆ haloalkyl, hydroxyl, C₁₋₆ alkoxy, and C₁₋₆ haloalkoxy.

In certain embodiments, R¹ is halogen. In certain embodiments, R¹ is chloro or fluoro. In certain embodiments, R¹ is halogen, and m is 1.

In certain embodiments, R^{2B} is hydrogen or methyl. In certain embodiments, R^{2B} is hydrogen. In certain embodiments, R^{2B} is methyl.

In certain embodiments, R³ represents independently for each occurrence C₁₋₆ haloalkyl, halogen, C₁₋₆ alkyl, C₁₋₆ alkoxy, or —O—(C₁₋₆ alkylene)-OH. In certain embodiments, R³ is trifluoromethyl, difluoromethyl, fluoro, chloro, or methoxy. In certain embodiments, R³ is trifluoromethyl.

In certain embodiments, R⁴ and R⁵ each represent independently for each occurrence hydrogen or C₁₋₆ alkyl. In certain embodiments, R⁴ is hydrogen. In certain embodiments, R⁵ is hydrogen.

In certain embodiments, m is 0 or 1. In certain embodiments, m is 1. In certain embodiments, m is 0.

In certain embodiments, n is 1 or 2. In certain embodiments, n is 1.

The definitions of variables in Formula I-D above encompass multiple chemical groups. The application contemplates embodiments where, for example, i) the definition of a variable is a single chemical group selected from those chemical groups set forth above, ii) the definition of a variable is a collection of two or more of the chemical groups selected from those set forth above, and iii) the compound is defined by a combination of variables in which the variables are defined by (i) or (ii), e.g., such as where A is phenylene and R³ is selected from the group consisting of C₁₋₆ haloalkyl, halogen, hydroxyl, and C₁₋₆ alkyl.

Another aspect of the invention provides a compound represented by Formula II:

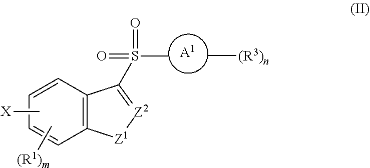

or a pharmaceutically acceptable salt thereof; wherein:

A¹ is phenylene, 5-6 membered heteroarylene, or 3-6 membered heterocycloalkylene;

Z¹ and Z² are defined by:
(i) Z¹ is O or —N(R^{2B})—; and Z² is C(R^{2A}); or
(ii) Z¹ is —N(R^{2A})—; and Z² is N;

X is phenyl or 5-10 membered heteroaryl, each of which is optionally substituted by 1, 2, 3, or 4 substituents independently selected from the group consisting of C₁₋₆ alkyl, C₃₋₆ cycloalkyl, halogen, C₁₋₆ haloalkyl, hydroxyl, C₁₋₆ alkoxy, C₁₋₆ haloalkoxy, —O—C₃₋₆ cycloalkyl, —O—(C₁₋₆ alkylene)-OH, cyano, —N(R⁴)(R⁵), —S(O)₂—R⁶, acetyl, and C₆₋₁₀ aryl;

R¹ represents independently for each occurrence halogen, C₁₋₆ alkyl, C₁₋₆ haloalkyl, or C₃₋₆ cycloalkyl;

R^{2A} is —(C₁₋₆ alkylene)-A², —(C₃₋₆ cycloalkylene)-A², -(2-6 membered heteroalkylene)-A², —(C₁₋₃ alkylene)-(C₃₋₆ cycloalkylene)-(C₀₋₃ alkylene)-A², —(C₁₋₃ alkylene)-(3-6 membered heterocycloalkylene)-(C₀₋₃ alkylene)-A², —(C₆₋₁₀ arylene)-A², C₂₋₆ hydroxyalkyl, —CO₂R⁴, or hydrogen; wherein:
A² is —CO₂R⁴, —C(O)-A³, —C(O)R⁶, —C(O)N(R⁴)(R⁵), —C(O)N(R⁴)—(C₁₋₄ alkylene)-CO₂R⁴, —C(O)N(R⁴)SO₂R⁴, —C(O)N(R⁴)SO₂A³, —C(O)N(R⁴)—(C₁₋₆ alkylene)-N(R⁷)C(O)R⁶, —C(O)N(R⁴)—(C₁₋₆ alkylene)-SO₂N(R)₂, —C(O)N(R⁴)—(C₁₋₆ alkylene)-CN, —C(O)N(R⁴)—(C₁₋₆ alkylene)-OC(O)R⁶, —N(R⁴)C(O)R⁷, —N(R⁴)C(O)A³, —N(R⁴)C(O)—(C₁₋₆ alkylene)-CO₂R⁴, —N(R⁴)C(O)—(C₁₋₆ alkylene)-N(R⁷)C(O)R⁶, —N(R⁴)C(O)—(C₁₋₆ alkylene)-SO₂N(R⁷)₂, —N(R⁴)C(O)—(C₁₋₆ alkylene)-CN, —N(R⁴)C(O)—(C₁₋₆ alkylene)-OC(O)R⁶, —N(R⁴)C(O)N(R⁴)—(C₁₋₆ alkylene)-CO₂R⁴, —N(R⁴)C(O)N(R⁷)₂, —N(R⁴)CO₂R⁶, —N(R⁴)S(O)₂R⁷, —N(R⁴)S(O)₂N(R⁴)(R⁵), —N(R⁴)(R⁵), hydroxyl, or A³;
A³ is aryl, C₃₋₆ cycloalkyl, or a 5-8 membered heterocyclic group, each of which is optionally substituted with 1, 2, or 3 substituents independently selected from the group consisting of C₁₋₆ alkyl, C₁₋₆ haloalkyl, C₃₋₆ cycloalkyl, halogen, C₁₋₆ alkoxy, C₁₋₆ haloalkoxy, oxo, —C(O)R⁶, —CO₂R⁷, —C(O)N(R⁴)(R⁵), —N(R⁴)C(O)(R⁶), and —N(R⁴)(R⁵); and
any alkylene, cycloalkylene, or heteroalkylene within the definition of R^{2A} is optionally substituted by 1, 2, or 3 substitutents independently selected from the group consisting of hydroxyl and C₁₋₆ alkoxy;

R^{2B} represents independently for each occurrence hydrogen, C₁₋₆ alkyl, or C₁₋₃ haloalkyl;

R³ represents independently for each occurrence hydrogen, C₁₋₆ haloalkyl, halogen, hydroxyl, C₁₋₆ alkyl, C₃₋₆ cycloalkyl, C₁₋₆ alkoxy, C₁₋₆ haloalkoxy, —O—(C₁₋₆ alkylene)-OH, or —O—(C₁₋₆ alkylene)-CO₂R⁴; or two vicinal occurrences of R³ are taken together with intervening atoms to form a 4-6 membered ring optionally substituted with 1, 2, or 3 substituents independently selected from the group consisting of C₁₋₆ alkyl, C₁₋₆ haloalkyl, C₃₋₆ cycloalkyl, halogen, C₁₋₆ alkoxy, C₁₋₆ haloalkoxy, —C(O)R⁶, and —CO₂R⁷;

R⁴ and R⁵ each represent independently for each occurrence hydrogen, C₁₋₆ alkyl, C₁₋₆ haloalkyl, C₁₋₆ hydroxyalkyl, or C₃₋₆ cycloalkyl; or an occurrence of R⁴ and R⁵ attached to the same nitrogen atom are taken together with the nitrogen atom to which they are attached to form a 3-7 membered heterocyclic ring;

R⁶ represents independently for each occurrence C₁₋₆ alkyl, C₃₋₆ cycloalkyl, or aralkyl;

R⁷ represents independently for each occurrence hydrogen, C₁₋₆ alkyl, C₁₋₆ hydroxyalkyl, C₁₋₆ haloalkyl, C₁₋₆ hydroxyhaloalkyl, C₃₋₆ cycloalkyl, C₃₋₆ hydroxycycloalkyl, —(C₁₋₆ alkylene)-(C₃₋₆ cycloalkyl), —(C₁₋₆ alkylene)-(C₂₋₄ alkenyl), or aralkyl;

m is 0, 1, or 2; and

n is 1, 2, or 3.

In certain embodiments, the compound is represented by Formula II.

In certain embodiments, A¹ is phenylene or 5-6 membered heteroarylene. In certain embodiments, A¹ is phenylene. In certain embodiments, A¹ is 5-6 membered heteroarylene. In certain embodiments, -A¹-(R³)ₙ is

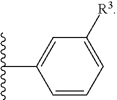

In certain embodiments, Z¹ is O or —N(R^{2B})— and Z² is C(R^{2A}). In certain embodiments, Z¹ is O and Z² is C(R^{2A}). In certain embodiments, Z¹ is —N(R^{2B})— and Z² is C(R^{2A}). In certain embodiments, Z¹ is —N(R^{2A})— and Z² is N.

In certain embodiments, X is phenyl or 5-6 membered heteroaryl, each of which is optionally substituted by 1, 2, or 3 substituents independently selected from the group consisting of C₁₋₆ alkyl, C₃₋₆ cycloalkyl, halogen, C₁₋₆ haloalkyl, hydroxyl, C₁₋₆ alkoxy, C₁₋₆ haloalkoxy, —O—C₃₋₆ cycloalkyl, —O—(C₁₋₆ alkylene)-OH, cyano, —N(R⁴)(R⁵), and C₆₋₁₀ aryl. In certain embodiments, X is phenyl substituted by 1, 2, or 3 substituents independently selected from the group consisting of C₁₋₆ alkyl, C₃₋₆ cycloalkyl, halogen, C₁₋₆ haloalkyl, hydroxyl, C₁₋₆ alkoxy, C₁₋₆ haloalkoxy, —O—C₃₋₆ cycloalkyl, and —O—(C₁₋₆ alkylene)-OH. In certain embodiments, X is phenyl substituted by 1, 2, or 3 substituents independently selected from the group consisting of C₁₋₆ alkyl, C₃₋₆ cycloalkyl, halogen, C₁₋₆ haloalkyl, hydroxyl, C₁₋₆ alkoxy, and C₁₋₆ haloalkoxy. In certain embodiments, X is phenyl substituted by 1, 2, or 3 substituents independently selected from the group consisting of C₁₋₆ alkyl, cyclopropyl, halogen, C₁₋₃ haloalkyl, hydroxyl, C₁₋₃ alkoxy, and C₁₋₃ haloalkoxy. In certain embodiments, X is phenyl substituted by 1, 2, or 3 substituents independently selected from the group consisting of halogen, C₁₋₃ haloalkyl, C₁₋₃ alkoxy, and C₁₋₃ haloalkoxy.

In certain embodiments, X is a 5-6 membered heteroaryl optionally substituted by 1, 2, or 3 substituents independently selected from the group consisting of C₁₋₆ alkyl, C₃₋₆ cycloalkyl, halogen, C₁₋₆ haloalkyl, hydroxyl, C₁₋₆ alkoxy, C₁₋₆ haloalkoxy, —O—C₃₋₆ cycloalkyl, —O—(C₁₋₆ alkylene)-OH, cyano, —N(R⁴)(R⁵), and C₆₋₁₀ aryl. In certain embodiments, X is a 6-membered heteroaryl substituted by 1, 2, or 3 substituents independently selected from the group consisting of C₁₋₆ alkyl, C₃₋₆ cycloalkyl, halogen, C₁₋₆ haloalkyl, hydroxyl, C₁₋₆ alkoxy, and C₁₋₆ haloalkoxy. In certain embodiments, X is a 6-membered heteroaryl substituted by 1, 2, or 3 substituents independently selected from the group consisting of halogen, C₁₋₆ haloalkyl, C₁₋₆ alkoxy, and C₁₋₆ haloalkoxy. In certain embodiments, X is pyridinyl, pyrimidinyl, pyrazolyl, imidazolyl, triazolyl, thiazolyl, oxazolyl, or thiadiazolyl, each of which is optionally substituted by 1, 2, or 3 substituents independently selected from the group consisting of C₁₋₆ alkyl, C₃₋₆ cycloalkyl, halogen, C₁₋₆ haloalkyl, hydroxyl, C₁₋₆ alkoxy, and C₁₋₆ haloalkoxy.

In certain embodiments, R¹ represents independently for each occurrence halogen, C₁₋₆ alkyl, or C₁₋₆ haloalkyl. In certain embodiments, R¹ is halogen. In certain embodiments, R¹ is chloro or fluoro. In certain embodiments, R¹ is halogen, and m is 1.

In certain embodiments, R^{2A} is —(C₁₋₆ alkylene)-A², —(C₃₋₆ cycloalkylene)-A², -(2-6 membered heteroalkylene)-A², —(C₁₋₃ alkylene)-(C₃₋₆ cycloalkylene)-(C₀₋₃ alkylene)-A², —(C₁₋₃ alkylene)-(3-6 membered heterocycloalkylene)-(C₀₋₃ alkylene)-A², or —CO₂R⁴. In certain embodiments, R^{2A} is —(C₁₋₆ alkylene)-A². In certain embodiments, R^{2A} is —(C₂₋₃ alkylene)-A². In certain embodiments, R^{2A} is -(2-6 membered heteroalkylene)-A². In certain embodiments, R^{2A} is —(C₁₋₃ alkylene)-(C₃₋₆ cycloalkylene)-(C₀₋₃ alkylene)-A² or —(C₁₋₃ alkylene)-(3-6 membered heterocycloalkylene)-(C₀₋₃ alkylene)-A². In certain embodiments, R^{2A} is —CO₂R⁴.

In certain embodiments, A² is —CO₂R⁴, —C(O)-A³, —C(O)N(R⁴)(R⁵), —C(O)N(R⁴)—(C₁₋₄ alkylene)-CO₂R⁴, —N(R⁴)C(O)R⁷, —N(R⁴)C(O)-A³, —N(R⁴)C(O)—(C₁₋₆ alkylene)-CO₂R⁴, —N(R⁴)C(O)N(R⁴)—(C₁₋₆ alkylene)-CO₂R⁴, —N(R⁴)CO₂R⁶, or A³. In certain embodiments, A² is —CO₂R⁴. In certain embodiments, A² is —CO₂H. In certain embodiments, A² is —C(O)-A³.

In certain embodiments, R^{2A} is —(C₁₋₆ alkylene)-A², and A² is —C(O)N(R⁴)—(C₁₋₄ alkylene)-CO₂R⁴, —C(O)N(R⁴)SO₂R⁴, or —C(O)N(R⁴)SO₂A³. In certain embodiments, R^{2A} is —(C₁₋₆ alkylene)-A², and A² is —C(O)N(R⁴)—(C₁₋₆ alkylene)-N(R⁷)C(O)R⁶, —C(O)N(R⁴)—(C₁₋₆ alkylene)-SO₂N(R⁷)₂, —C(O)N(R⁴)—(C₁₋₆ alkylene)-CN, or —C(O)N(R⁴)—(C₁₋₆ alkylene)-OC(O)R⁶. In certain embodiments, R^{2A} is —(C₁₋₆ alkylene)-A², and A² is —N(R⁴)C(O)R⁷, —N(R⁴)C(O)A³, —N(R⁴)C(O)—(C₁₋₆ alkylene)-CO₂R⁴, —N(R⁴)C(O)—(C₁₋₆ alkylene)-N(R⁷)C(O)R⁶, —N(R⁴)C(O)—(C₁₋₆ alkylene)-SO₂N(R⁷)₂, —N(R⁴)C(O)—(C₁₋₆ alkylene)-CN, —N(R⁴)C(O)—(C₁₋₆ alkylene)-OC(O)R⁶, or —N(R⁴)C(O)N(R⁴)—(C₁₋₆ alkylene)-CO₂R⁴. In certain embodiments, R^{2A} is —(C₁₋₆ alkylene)-A², and A² is —N(R⁴)C(O)N(R⁷)₂, —N(R⁴)CO₂R⁶, —N(R⁴)S(O)₂R⁷, or —N(R⁴)S(O)₂N(R⁴)(R⁵). In certain embodiments, R^{2A} is —(C₁₋₆ alkylene)-A², and A² is —N(R⁴)(R⁵) or hydroxyl. In certain embodiments, R^{2A} is —(C₁₋₆ alkylene)-A², and A² is A³.

In certain embodiments, A³ is a 5-8 membered heterocyclic group optionally substituted with 1, 2, or 3 substituents independently selected from the group consisting of C₁₋₆ alkyl, C₁₋₆ haloalkyl, C₃₋₆ cycloalkyl, halogen, C₁₋₆ alkoxy, C₁₋₆ haloalkoxy, oxo, —C(O)R⁶, —CO₂R⁷, and —C(O)N(R⁴)(R⁵). In certain embodiments, A³ is a 5-6 membered heterocyclic group optionally substituted with 1, 2, or 3 substituents independently selected from the group consisting of C₁₋₆ alkyl, C₁₋₆ haloalkyl, halogen, C₁₋₆ alkoxy, oxo, —C(O)R⁶, and —CO₂R⁷. In certain embodiments, A³ is a 5-6 membered saturated heterocyclic group optionally substituted by 1 or 2 substituents independently selected from the group consisting of C₁₋₆ alkyl, C₁₋₆ haloalkyl, C₃₋₆ cycloalkyl, halogen, C₁₋₆ alkoxy, C₁₋₆ haloalkoxy, oxo, —C(O)R⁶, —CO₂R⁷, and —C(O)N(R⁴)(R⁵). In certain embodiments, A³ is a 5-6 membered saturated heterocyclic group optionally substituted by 1 or 2 substituents independently selected from the group consisting of methyl, ethyl, isopropyl, trifluoromethyl, cyclopropyl, C₁₋₆ alkoxy, oxo, and —CO₂R⁷. In certain embodiments, A³ is a 5-6 membered heterocyclic group containing at least one ring carbon atom substituted by oxo and the heterocyclic group being optionally substituted by 1 or 2 substituents independently selected from the group consisting of C₁₋₆ alkyl, C₁₋₆ haloalkyl, C₃₋₆ cycloalkyl, halogen, C₁₋₆ alkoxy, C₁₋₆ haloalkoxy, oxo, —C(O)R⁶, —CO₂R⁷, and —C(O)N(R⁴)(R⁵).

In certain embodiments, R^{2B} is hydrogen or methyl. In certain embodiments, R^{2B} is hydrogen. In certain embodiments, R^{2B} is methyl.

In certain embodiments, R³ represents independently for each occurrence hydrogen, C₁₋₆ haloalkyl, halogen, hydroxyl, C₁₋₆ alkyl, C₃₋₆ cycloalkyl, C₁₋₆ alkoxy, C₁₋₆ haloalkoxy, —O—(C₁₋₆ alkylene)-OH, or —O—(C₁₋₆ alkylene)-CO₂R⁴. In certain embodiments, R³ represents independently for each occurrence C₁₋₆ haloalkyl, halogen, C₁₋₆ alkyl, C₁₋₆ alkoxy, or —O—(C₁₋₆ alkylene)-OH. In certain embodiments, R³ is trifluoromethyl, difluoromethyl, fluoro, chloro, or methoxy. In certain embodiments, R³ is trifluoromethyl.

In certain embodiments, R⁴ and R⁵ each represent independently for each occurrence hydrogen, C₁₋₆ alkyl, or C₃₋₆ cycloalkyl; or an occurrence of R⁴ and R⁵ attached to the same nitrogen atom are taken together with the nitrogen atom to which they are attached to form a 3-7 membered heterocyclic ring. In certain embodiments, R⁴ and R⁵ each represent independently for each occurrence hydrogen or C₁₋₆ alkyl. In certain embodiments, R⁴ is hydrogen. In certain embodiments, R⁵ is hydrogen.

In certain embodiments, R⁶ represents independently for each occurrence C₁₋₆ alkyl or C₃₋₆ cycloalkyl. In certain embodiments, R⁶ is C₁₋₆ alkyl.

In certain embodiments, R⁷ represents independently for each occurrence hydrogen, C₁₋₆ alkyl, C₁₋₆ hydroxyalkyl, C₃₋₆ cycloalkyl, C₃₋₆ hydroxycycloalkyl, or aralkyl. In certain embodiments, R⁷ represents independently for each occurrence hydrogen, C₁₋₆ alkyl, or C₁₋₆ hydroxyalkyl. In certain embodiments, R⁷ is hydrogen. In certain embodiments, R⁷ is C₁₋₆ alkyl.

In certain embodiments, m is 0 or 1. In certain embodiments, m is 1. In certain embodiments, m is 0.

In certain embodiments, n is 1 or 2. In certain embodiments, n is 1.

The definitions of variables in Formula II above encompass multiple chemical groups. The application contemplates embodiments where, for example, i) the definition of a variable is a single chemical group selected from those chemical groups set forth above, ii) the definition of a variable is a collection of two or more of the chemical groups selected from those set forth above, and iii) the compound is defined by a combination of variables in which the variables are defined by (i) or (ii), e.g., such as where A is phenylene and R³ is selected from the group consisting of C₁₋₆ haloalkyl, halogen, hydroxyl, and C₁₋₆ alkyl.

Another aspect of the invention provides a compound represented by Formula II-A:

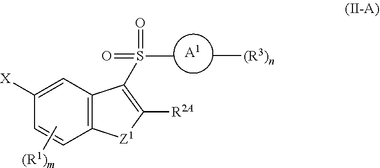

or a pharmaceutically acceptable salt thereof; wherein:

A¹ is phenylene or a 5-6 membered heteroarylene;

Z¹ is O or —N(R^{2B})—;

X is phenyl or 5-6 membered heteroaryl, each of which is optionally substituted by 1, 2, or 3 substituents independently selected from the group consisting of C₁₋₆ alkyl, C₃₋₆ cycloalkyl, halogen, C₁₋₆ haloalkyl, hydroxyl, C₁₋₆ alkoxy, C₁₋₆ haloalkoxy, —O—C₃₋₆ cycloalkyl, —O—(C₁₋₆ alkylene)-OH, cyano, —N(R⁴)(R⁵), and C₆₋₁₀ aryl;

R¹ represents independently for each occurrence halogen, C₁₋₆ alkyl, or C₁₋₆ haloalkyl;

R^{2A} is —(C₁₋₆ alkylene)-A², —(C₃₋₆ cycloalkylene)-A², -(2-6 membered heteroalkylene)-A², —(C₁₋₃ alkylene)-(C₃₋₆ cycloalkylene)-(C₀₋₃ alkylene)-A², —(C₁₋₃ alkylene)-(3-6 membered heterocycloalkylene)-(C₀₋₃ alkylene)-A², or —CO₂R⁴; wherein:
A² is —CO₂R⁴, —C(O)-A³, —C(O)N(R⁴)(R⁵), —C(O)N(R⁴)—(C₁₋₄ alkylene)-CO₂R⁴, —N(R⁴)C(O)R⁷, —N(R⁴)C(O)-A³, —N(R⁴)C(O)—(C₁₋₆ alkylene)-CO₂R⁴, —N(R⁴)C(O)N(R⁴)—(C₁₋₆ alkylene)-CO₂R⁴, —N(R⁴)CO₂R⁶, or A³; and
A³ is a 5-8 membered heterocyclic group optionally substituted with 1, 2, or 3 substituents independently selected from the group consisting of C₁₋₆ alkyl, C₁₋₆ haloalkyl, C₃₋₆ cycloalkyl, halogen, C₁₋₆ alkoxy, C₁₋₆ haloalkoxy, oxo, —C(O)R⁶, —CO₂R⁷, and —C(O)N(R⁴)(R⁵);

R^{2B} represents independently for each occurrence hydrogen, C₁₋₆ alkyl, or C₁₋₃ haloalkyl;

R³ represents independently for each occurrence hydrogen, C₁₋₆ haloalkyl, halogen, hydroxyl, C₁₋₆ alkyl, C₃₋₆ cycloalkyl, C₁₋₆ alkoxy, C₁₋₆ haloalkoxy, —O—(C₁₋₆ alkylene)-OH, or —O—(C₁₋₆ alkylene)-CO₂R⁴;

R⁴ and R⁵ each represent independently for each occurrence hydrogen, C₁₋₆ alkyl, or C₃₋₆ cycloalkyl; or an occurrence of R⁴ and R⁵ attached to the same nitrogen atom are taken together with the nitrogen atom to which they are attached to form a 3-7 membered heterocyclic ring;

R⁶ represents independently for each occurrence C₁₋₆ alkyl, C₃₋₆ cycloalkyl, or aralkyl;

R⁷ represents independently for each occurrence hydrogen, C₁₋₆ alkyl, C₁₋₆ hydroxyalkyl, C₃₋₆ cycloalkyl, C₃₋₆ hydroxycycloalkyl, or aralkyl;

m is 0, 1, or 2; and

n is 1, 2, or 3.

In certain embodiments, the compound is represented by Formula II-A.

In certain embodiments, A¹ is phenylene. In certain embodiments, A¹ is 5-6 membered heteroarylene. In certain embodiments, -A¹-(R³)ₙ is

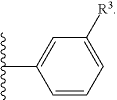

In certain embodiments, Z¹ is O. In certain embodiments, Z¹ is —N(R^{2B})—.

In certain embodiments, X is phenyl substituted by 1, 2, or 3 substituents independently selected from the group consisting of C₁₋₆ alkyl, C₃₋₆ cycloalkyl, halogen, C₁₋₆ haloalkyl, hydroxyl, C₁₋₆ alkoxy, C₁₋₆ haloalkoxy, —O—C₃₋₆ cycloalkyl, and —O—(C₁₋₆ alkylene)-OH. In certain embodiments, X is phenyl substituted by 1, 2, or 3 substituents independently selected from the group consisting of C₁₋₆ alkyl, C₃₋₆ cycloalkyl, halogen, C₁₋₆ haloalkyl, hydroxyl, C₁₋₆ alkoxy, and C₁₋₆ haloalkoxy. In certain embodiments, X is phenyl substituted by 1, 2, or 3 substituents independently selected from the group consisting of C₁₋₆ alkyl, cyclopropyl, halogen, C₁₋₃ haloalkyl, hydroxyl, C₁₋₃ alkoxy, and C₁₋₃ haloalkoxy. In certain embodiments, X is phenyl substituted by 1, 2, or 3 substituents independently selected from the group consisting of halogen, C₁₋₃ haloalkyl, C₁₋₃ alkoxy, and C₁₋₃ haloalkoxy.

In certain embodiments, X is a 5-6 membered heteroaryl optionally substituted by 1, 2, or 3 substituents independently selected from the group consisting of C₁₋₆ alkyl, C₃₋₆ cycloalkyl, halogen, C₁₋₆ haloalkyl, hydroxyl, C₁₋₆ alkoxy, C₁₋₆ haloalkoxy, —O—C₃₋₆ cycloalkyl, —O—(C₁₋₆ alkylene)-OH, cyano, —N(R⁴)(R⁵), and C₆₋₁₀ aryl. In certain embodiments, X is a 6-membered heteroaryl substituted by 1, 2, or 3 substituents independently selected from the group consisting of C₁₋₆ alkyl, C₃₋₆ cycloalkyl, halogen, C₁₋₆ haloalkyl, hydroxyl, C₁₋₆ alkoxy, and C₁₋₆ haloalkoxy. In certain embodiments, X is a 6-membered heteroaryl substituted by 1, 2, or 3 substituents independently selected from the group consisting of halogen, C₁₋₆ haloalkyl, C₁₋₆ alkoxy, and C₁₋₆ haloalkoxy. In certain embodiments, X is pyridinyl, pyrimidinyl, pyrazolyl, imidazolyl, triazolyl, thiazolyl, oxazolyl, or thiadiazolyl, each of which is optionally substituted by 1, 2, or 3 substituents independently selected from the group consisting of C₁₋₆ alkyl, C₃₋₆ cycloalkyl, halogen, C₁₋₆ haloalkyl, hydroxyl, C₁₋₆ alkoxy, and C₁₋₆ haloalkoxy.

In certain embodiments, R¹ is halogen. In certain embodiments, R¹ is chloro or fluoro. In certain embodiments, R¹ is halogen, and m is 1.

In certain embodiments, R^{2A} is —(C₁₋₆ alkylene)-A². In certain embodiments, R^{2A} is —(C₂₋₃ alkylene)-A². In certain embodiments, R^{2A} is -(2-6 membered heteroalkylene)-A². In certain embodiments, R^{2A} is —(C₁₋₃ alkylene)-(C₃₋₆ cycloalkylene)-(C₀₋₃ alkylene)-A² or —(C₁₋₃ alkylene)-(3-6 membered heterocycloalkylene)-(C₀₋₃ alkylene)-A². In certain embodiments, R^{2A} is —CO₂R⁴.

In certain embodiments, A² is —CO₂R⁴. In certain embodiments, A² is —CO₂H. In certain embodiments, A² is —C(O)-A³.

In certain embodiments, A³ is a 5-6 membered heterocyclic group optionally substituted with 1, 2, or 3 substituents independently selected from the group consisting of C₁₋₆ alkyl, C₁₋₆ haloalkyl, halogen, C₁₋₆ alkoxy, oxo, —C(O)R⁶, and —CO₂R⁷. In certain embodiments, A³ is a 5-6 membered saturated heterocyclic group optionally substituted by 1 or 2 substituents independently selected from the group consisting of C₁₋₆ alkyl, C₁₋₆ haloalkyl, C₃₋₆ cycloalkyl, halogen, C₁₋₆ alkoxy, C₁₋₆ haloalkoxy, oxo, —C(O)R⁶, —CO₂R⁷, and —C(O)N(R⁴)(R⁵). In certain embodiments, A³ is a 5-6 membered saturated heterocyclic group optionally substituted by 1 or 2 substituents independently selected from the group consisting of methyl, ethyl, isopropyl, trifluoromethyl, cyclopropyl, C₁₋₆ alkoxy, oxo, and —CO₂R⁷. In certain embodiments, A³ is a 5-6 membered heterocyclic group containing at least one ring carbon atom substituted by oxo and the heterocyclic group being optionally substituted by 1 or 2 substituents independently selected from the group consisting of C₁₋₆ alkyl, C₁₋₆ haloalkyl, C₃₋₆ cycloalkyl, halogen, C₁₋₆ alkoxy, C₁₋₆ haloalkoxy, oxo, —C(O)R⁶, —CO₂R⁷, and —C(O)N(R⁴)(R⁵).

In certain embodiments, R^{2B} is hydrogen or methyl. In certain embodiments, R^{2B} is hydrogen. In certain embodiments, R^{2B} is methyl.

In certain embodiments, R³ represents independently for each occurrence C₁₋₆ haloalkyl, halogen, C₁₋₆ alkyl, C₁₋₆ alkoxy, or —O—(C₁₋₆ alkylene)-OH. In certain embodiments, R³ is trifluoromethyl, difluoromethyl, fluoro, chloro, or methoxy. In certain embodiments, R³ is trifluoromethyl.

In certain embodiments, R⁴ and R⁵ each represent independently for each occurrence hydrogen or C₁₋₆ alkyl. In certain embodiments, R⁴ is hydrogen. In certain embodiments, R⁵ is hydrogen.

In certain embodiments, R⁶ represents independently for each occurrence C₁₋₆ alkyl or C₃₋₆ cycloalkyl. In certain embodiments, R⁶ is C₁₋₆ alkyl.

In certain embodiments, R⁷ represents independently for each occurrence hydrogen, C₁₋₆ alkyl, or C₁₋₆ hydroxyalkyl. In certain embodiments, R⁷ is hydrogen. In certain embodiments, R⁷ is C₁₋₆ alkyl.

In certain embodiments, m is 0 or 1. In certain embodiments, m is 1. In certain embodiments, m is 0.

In certain embodiments, n is 1 or 2. In certain embodiments, n is 1.

The definitions of variables in Formula II-A above encompass multiple chemical groups. The application contemplates embodiments where, for example, i) the definition of a variable is a single chemical group selected from those chemical groups set forth above, ii) the definition of a variable is a collection of two or more of the chemical groups selected from those set forth above, and iii) the compound is defined by a combination of variables in which the variables are defined by (i) or (ii), e.g., such as where A is phenylene and R³ is selected from the group consisting of C₁₋₆ haloalkyl, halogen, hydroxyl, and C₁₋₆ alkyl.

Another aspect of the invention provides a compound represented by Formula II-B:

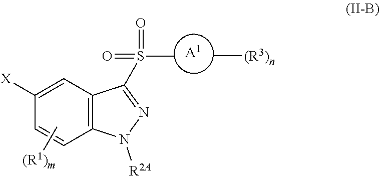

or a pharmaceutically acceptable salt thereof; wherein:

A¹ is phenylene or a 5-6 membered heteroarylene;

X is phenyl or 5-6 membered heteroaryl, each of which is optionally substituted by 1, 2, or 3 substituents independently selected from the group consisting of C₁₋₆ alkyl, C₃₋₆ cycloalkyl, halogen, C₁₋₆ haloalkyl, hydroxyl, C₁₋₆ alkoxy, C₁₋₆ haloalkoxy, —O—C₃₋₆ cycloalkyl, —O—(C₁₋₆ alkylene)-OH, cyano, —N(R⁴)(R⁵), and C₆₋₁₀ aryl;

R¹ represents independently for each occurrence halogen, C₁₋₆ alkyl, or C₁₋₆ haloalkyl;

R^{2A} is —(C₁₋₆ alkylene)-A², —(C₃₋₆ cycloalkylene)-A², -(2-6 membered heteroalkylene)-A², —(C₁₋₃ alkylene)-(C₃₋₆ cycloalkylene)-(C₀₋₃ alkylene)-A², —(C₁₋₃ alkylene)-(3-6 membered heterocycloalkylene)-(C₀₋₃ alkylene)-A², or —CO₂R⁴; wherein:
A² is —CO₂R⁴, —C(O)-A³, —C(O)N(R⁴)(R⁵), —C(O)N(R⁴)—(C₁₋₄ alkylene)-CO₂R⁴, —N(R⁴)C(O)R⁷, —N(R⁴)C(O)-A³, —N(R⁴)C(O)—(C₁₋₆ alkylene)-CO₂R⁴, —N(R⁴)C(O)N(R⁴)—(C₁₋₆ alkylene)-CO₂R⁴, —N(R⁴)CO₂R⁶, or A³; and
A³ is a 5-8 membered heterocyclic group optionally substituted with 1, 2, or 3 substituents independently selected from the group consisting of C₁₋₆ alkyl, C₁₋₆ haloalkyl, C₃₋₆ cycloalkyl, halogen, C₁₋₆ alkoxy, C₁₋₆ haloalkoxy, oxo, —C(O)R⁶, —CO₂R⁷, and —C(O)N(R⁴)(R⁵);

R³ represents independently for each occurrence hydrogen, C₁₋₆ haloalkyl, halogen, hydroxyl, C₁₋₆ alkyl, C₃₋₆ cycloalkyl, C₁₋₆ alkoxy, C₁₋₆ haloalkoxy, —O—(C₁₋₆ alkylene)-OH, or —O—(C₁₋₆ alkylene)-CO₂R⁴;

R⁴ and R⁵ each represent independently for each occurrence hydrogen, C₁₋₆ alkyl, or C₃₋₆ cycloalkyl; or an occurrence of R⁴ and R⁵ attached to the same nitrogen atom are taken together with the nitrogen atom to which they are attached to form a 3-7 membered heterocyclic ring;

R⁶ represents independently for each occurrence C₁₋₆ alkyl, C₃₋₆ cycloalkyl, or aralkyl;

R⁷ represents independently for each occurrence hydrogen, C₁₋₆ alkyl, C₁₋₆ hydroxyalkyl, C₃₋₆ cycloalkyl, C₃₋₆ hydroxycycloalkyl, or aralkyl;

m is 0, 1, or 2; and

n is 1, 2, or 3.

In certain embodiments, the compound is represented by Formula II-B.

In certain embodiments, A¹ is phenylene. In certain embodiments, A¹ is 5-6 membered heteroarylene. In certain embodiments, -A¹-(R³)ₙ

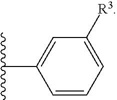

In certain embodiments, X is phenyl substituted by 1, 2, or 3 substituents independently selected from the group consisting of C₁₋₆ alkyl, C₃₋₆ cycloalkyl, halogen, C₁₋₆ haloalkyl, hydroxyl, C₁₋₆ alkoxy, C₁₋₆ haloalkoxy, —O—C₃₋₆ cycloalkyl, and —O—(C₁₋₆ alkylene)-OH. In certain embodiments, X is phenyl substituted by 1, 2, or 3 substituents independently selected from the group consisting of C₁₋₆ alkyl, C₃₋₆ cycloalkyl, halogen, C₁₋₆ haloalkyl, hydroxyl, C₁₋₆ alkoxy, and C₁₋₆ haloalkoxy. In certain embodiments, X is phenyl substituted by 1, 2, or 3 substituents independently selected from the group consisting of C₁₋₆ alkyl, cyclopropyl, halogen, C₁₋₃ haloalkyl, hydroxyl, C₁₋₃ alkoxy, and C₁₋₃ haloalkoxy. In certain embodiments, X is phenyl substituted by 1, 2, or 3 substituents independently selected from the group consisting of halogen, C₁₋₃ haloalkyl, C₁₋₃ alkoxy, and C₁₋₃ haloalkoxy.

In certain embodiments, X is a 5-6 membered heteroaryl optionally substituted by 1, 2, or 3 substituents independently selected from the group consisting of C₁₋₆ alkyl, C₃₋₆ cycloalkyl, halogen, C₁₋₆ haloalkyl, hydroxyl, C₁₋₆ alkoxy, C₁₋₆ haloalkoxy, —O—C₃-cycloalkyl, —O—(C₁₋₆ alkylene)-OH, cyano, —N(R⁴)(R⁵), and C₆₋₁₀ aryl. In certain embodiments, X is a 6-membered heteroaryl substituted by 1, 2, or 3 substituents independently selected from the group consisting of C₁₋₆ alkyl, C₃₋₆ cycloalkyl, halogen, C₁₋₆ haloalkyl, hydroxyl, C₁₋₆ alkoxy, and C₁₋₆ haloalkoxy. In certain embodiments, X is a 6-membered heteroaryl substituted by 1, 2, or 3 substituents independently selected from the group consisting of halogen, C₁₋₆ haloalkyl, C₁₋₆ alkoxy, and C₁₋₆ haloalkoxy. In certain embodiments, X is pyridinyl, pyrimidinyl, pyrazolyl, imidazolyl, triazolyl, thiazolyl, oxazolyl, or thiadiazolyl, each of which is optionally substituted by 1, 2, or 3 substituents independently selected from the group consisting of C₁₋₆ alkyl, C₃₋₆ cycloalkyl, halogen, C₁₋₆ haloalkyl, hydroxyl, C₁₋₆ alkoxy, and C₁₋₆ haloalkoxy.

In certain embodiments, R¹ is halogen. In certain embodiments, R¹ is chloro or fluoro. In certain embodiments, R¹ is halogen, and m is 1.

In certain embodiments, R^{2A} is —(C₁₋₆ alkylene)-A². In certain embodiments, R^{2A} is —(C₂₋₃ alkylene)-A². In certain embodiments, R^{2A} is -(2-6 membered heteroalkylene)-A². In certain embodiments, R^{2A} is —(C₁₋₃ alkylene)-(C₃₋₆ cycloalkylene)-(C₀₋₃ alkylene)-A² or —(C₁₋₃ alkylene)-(3-6 membered heterocycloalkylene)-(C₀₋₃ alkylene)-A². In certain embodiments, R^{2A} is —CO₂R⁴.

In certain embodiments, A² is —CO₂R⁴. In certain embodiments, A² is —CO₂H. In certain embodiments, A² is —C(O)-A³.

In certain embodiments, A³ is a 5-6 membered heterocyclic group optionally substituted with 1, 2, or 3 substituents independently selected from the group consisting of C₁₋₆ alkyl, C₁₋₆ haloalkyl, halogen, C₁₋₆ alkoxy, oxo, —C(O)R⁶, and —CO₂R⁷. In certain embodiments, A³ is a 5-6 membered saturated heterocyclic group optionally substituted by 1 or 2 substituents independently selected from the group consisting of C₁₋₆ alkyl, C₁₋₆ haloalkyl, C₃₋₆ cycloalkyl, halogen, C₁₋₆ alkoxy, C₁₋₆ haloalkoxy, oxo, —C(O)R⁶, —CO₂R⁷, and —C(O)N(R⁴)(R⁵). In certain embodiments, A³ is a 5-6 membered saturated heterocyclic group optionally substituted by 1 or 2 substituents independently selected from the group consisting of methyl, ethyl, isopropyl, trifluoromethyl, cyclopropyl, C₁₋₆ alkoxy, oxo, and —CO₂R⁷. In certain embodiments, A³ is a 5-6 membered heterocyclic group containing at least one ring carbon atom substituted by oxo and the heterocyclic group being optionally substituted by 1 or 2 substituents independently selected from the group consisting of C₁₋₆ alkyl, C₁₋₆ haloalkyl, C₃₋₆ cycloalkyl, halogen, C₁₋₆ alkoxy, C₁₋₆ haloalkoxy, oxo, —C(O)R⁶, —CO₂R⁷, and —C(O)N(R⁴)(R⁵).

In certain embodiments, R³ represents independently for each occurrence C₁₋₆ haloalkyl, halogen, C₁₋₆ alkyl, C₁₋₆ alkoxy, or —O—(C₁₋₆ alkylene)-OH. In certain embodiments, R³ is trifluoromethyl, difluoromethyl, fluoro, chloro, or methoxy. In certain embodiments, R³ is trifluoromethyl.

In certain embodiments, R⁴ and R⁵ each represent independently for each occurrence hydrogen or C₁₋₆ alkyl. In certain embodiments, R⁴ is hydrogen. In certain embodiments, R⁵ is hydrogen.

In certain embodiments, R⁶ represents independently for each occurrence C₁₋₆ alkyl or C₃₋₆ cycloalkyl. In certain embodiments, R⁶ is C₁₋₆ alkyl.

In certain embodiments, R⁷ represents independently for each occurrence hydrogen, C₁₋₆ alkyl, or C₁₋₆ hydroxyalkyl. In certain embodiments, R⁷ is hydrogen. In certain embodiments, R⁷ is C₁₋₆ alkyl.

In certain embodiments, m is 0 or 1. In certain embodiments, m is 1. In certain embodiments, m is 0.

In certain embodiments, n is 1 or 2. In certain embodiments, n is 1.

The definitions of variables in Formula II-B above encompass multiple chemical groups. The application contemplates embodiments where, for example, i) the definition of a variable is a single chemical group selected from those chemical groups set forth above, ii) the definition of a variable is a collection of two or more of the chemical groups selected from those set forth above, and iii) the compound is defined by a combination of variables in which the variables are defined by (i) or (ii), e.g., such as where A is phenylene and R³ is selected from the group consisting of C₁₋₆ haloalkyl, halogen, hydroxyl, and C₁₋₆ alkyl.

In certain other embodiments, the compound is one of the compounds listed in Tables 1-9 below or a pharmaceutically acceptable salt thereof. In certain embodiments, the compound is one of the compounds listed in Tables 1-9 below.

TABLE 1

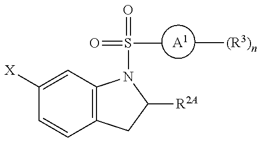

| No, | X | 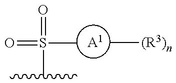 | R^{2A} |
|---|---|---|---|
| I-1 | 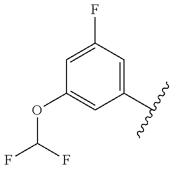 [SMILES: *c1cc(F)cc(OC(F)F)c1] | 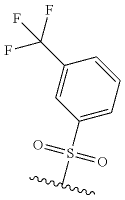 [SMILES: *S(=O)(=O)c1cccc(C(F)(F)F)c1] | H |
| I-2 | 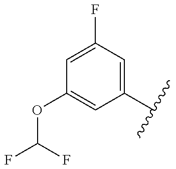 [SMILES: *c1cc(F)cc(OC(F)F)c1] | 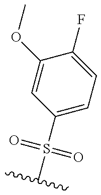 [SMILES: *S(=O)(=O)c1ccc(F)c(OC)c1] | 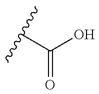 [SMILES: *C(=O)O] |
| I-3 | 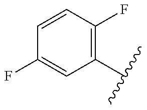 [SMILES: *c1cc(F)ccc1F] | 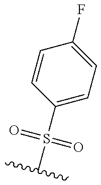 [SMILES: *S(=O)(=O)c1ccc(F)cc1] | 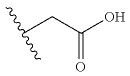 [SMILES: *CC(=O)O] |
| I-4 | 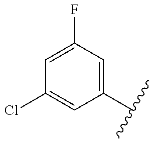 [SMILES: *c1cc(F)cc(Cl)c1] | 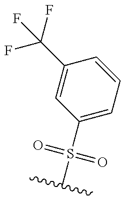 [SMILES: *S(=O)(=O)c1cccc(C(F)(F)F)c1] | 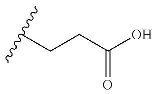 [SMILES: *CCC(=O)O] |
| I-5 | 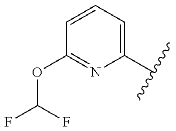 [SMILES: *c1cccc(OC(F)F)n1] | 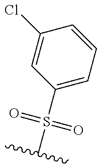 [SMILES: *S(=O)(=O)c1cccc(Cl)c1] | 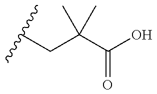 [SMILES: *CC(C)(C)C(=O)O] |
| I-6 | 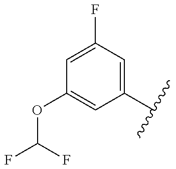 [SMILES: *c1cc(F)cc(OC(F)F)c1] | 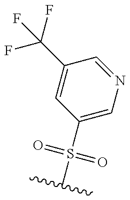 [SMILES: *S(=O)(=O)c1cncc(C(F)(F)F)c1] | 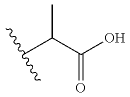 [SMILES: *C(C)C(=O)O] |
| I-7 | 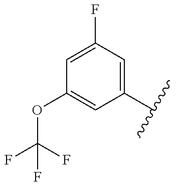 [SMILES: *c1cc(F)cc(OC(F)(F)F)c1] | 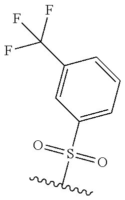 [SMILES: *S(=O)(=O)c1cccc(C(F)(F)F)c1] | 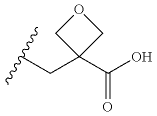 [SMILES: *CC1(C(=O)O)COC1] |
| I-8 | 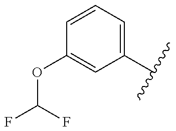 [SMILES: *c1cccc(OC(F)F)c1] | 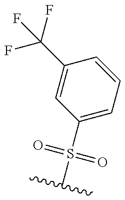 [SMILES: *S(=O)(=O)c1cccc(C(F)(F)F)c1] | 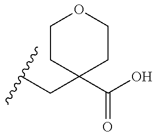 [SMILES: *CC1(C(=O)O)CCOCC1] |
| I-9 | 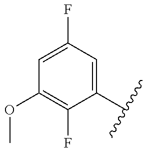 [SMILES: *c1cc(F)cc(OC)c1F] | 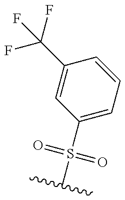 [SMILES: *S(=O)(=O)c1cccc(C(F)(F)F)c1] | 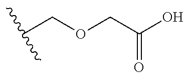 [SMILES: *COCC(=O)O] |
| I-10 | 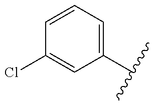 [SMILES: *c1cccc(Cl)c1] | 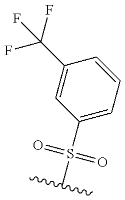 [SMILES: *S(=O)(=O)c1cccc(C(F)(F)F)c1] | 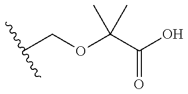 [SMILES: *COC(C)(C)C(=O)O] |

TABLE 2

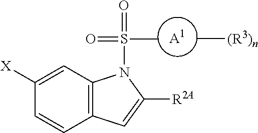

| No. | X | 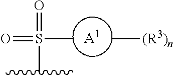 | R^{2A} |
|---|---|---|---|
| II-1 | 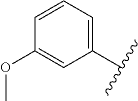 [SMILES: *c1cccc(OC)c1] | 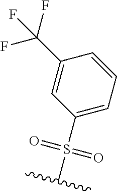 [SMILES: *S(=O)(=O)c1cccc(C(F)(F)F)c1] | 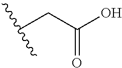 [SMILES: *CC(=O)O] |
| II-2 | 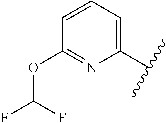 [SMILES: *c1cccc(OC(F)F)n1] | 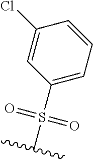 [SMILES: *S(=O)(=O)c1cccc(Cl)c1] | 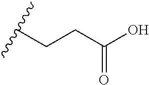 [SMILES: *CCC(=O)O] |
| II-3 | 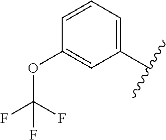 [SMILES: *c1cccc(OC(F)(F)F)c1] | 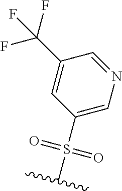 [SMILES: *S(=O)(=O)c1cncc(C(F)(F)F)c1] | 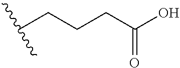 [SMILES: *CCCC(=O)O] |
| II-4 | 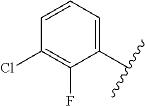 [SMILES: *c1cccc(Cl)c1F] | 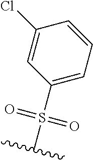 [SMILES: *S(=O)(=O)c1cccc(Cl)c1] | 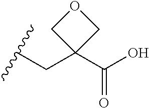 [SMILES: *CC1(C(=O)O)COC1] |
| II-5 | 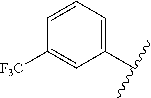 [SMILES: *c1cccc(C(F)(F)F)c1] | 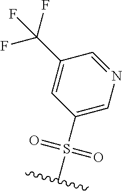 [SMILES: *S(=O)(=O)c1cncc(C(F)(F)F)c1] | 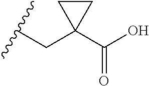 [SMILES: *CC1(C(=O)O)CC1] |
| II-6 | 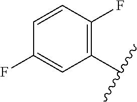 [SMILES: *c1cc(F)ccc1F] | 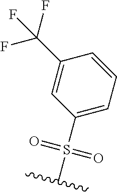 [SMILES: *S(=O)(=O)c1cccc(C(F)(F)F)c1] | 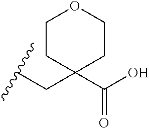 [SMILES: *CC1(C(=O)O)CCOCC1] |
| II-7 | 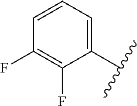 [SMILES: *c1cccc(F)c1F] | 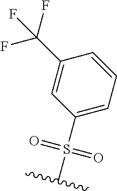 [SMILES: *S(=O)(=O)c1cccc(C(F)(F)F)c1] | 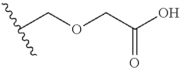 [SMILES: *COCC(=O)O] |
| II-8 | 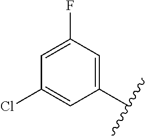 [SMILES: *c1cc(F)cc(Cl)c1] | 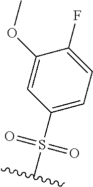 [SMILES: *S(=O)(=O)c1ccc(F)c(OC)c1] | 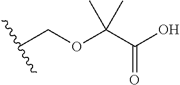 [SMILES: *COC(C)(C)C(=O)O] |
| II-9 | 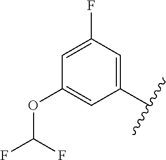 [SMILES: *c1cc(F)cc(OC(F)F)c1] | 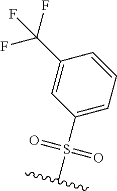 [SMILES: *S(=O)(=O)c1cccc(C(F)(F)F)c1] | 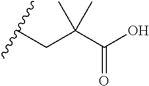 [SMILES: *CC(C)(C)C(=O)O] |
| II-10 | 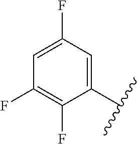 [SMILES: *c1cc(F)cc(F)c1F] | 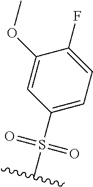 [SMILES: *S(=O)(=O)c1ccc(F)c(OC)c1] | 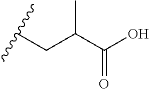 [SMILES: *CC(C)C(=O)O] |
| II-11 | 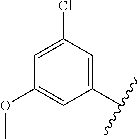 [SMILES: *c1cc(Cl)cc(OC)c1] | 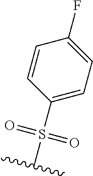 [SMILES: *S(=O)(=O)c1ccc(F)cc1] | 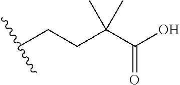 [SMILES: *CCC(C)(C)C(=O)O] |
| II-12 | 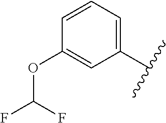 [SMILES: *c1cccc(OC(F)F)c1] | 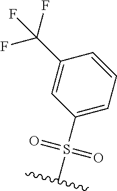 [SMILES: *S(=O)(=O)c1cccc(C(F)(F)F)c1] | 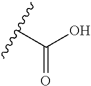 [SMILES: *C(=O)O] |

TABLE 3

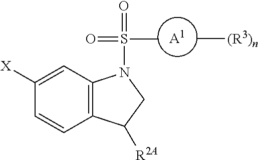

| No. | X | 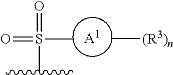 | R^{2A} |
|---|---|---|---|
| III-1 | 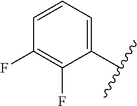 [SMILES: *c1cccc(F)c1F] | 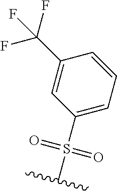 [SMILES: *S(=O)(=O)c1cccc(C(F)(F)F)c1] | 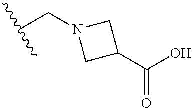 [SMILES: *CN1CC(C(=O)O)C1] |
| III-2 | 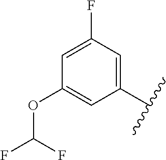 [SMILES: *c1cc(F)cc(OC(F)F)c1] | 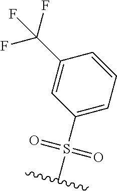 [SMILES: *S(=O)(=O)c1cccc(C(F)(F)F)c1] | 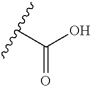 [SMILES: *C(=O)O] |
| III-3 | 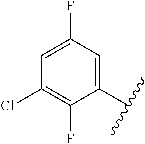 [SMILES: *c1cc(F)cc(Cl)c1F] | 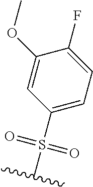 [SMILES: *S(=O)(=O)c1ccc(F)c(OC)c1] | 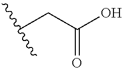 [SMILES: *CC(=O)O] |
| III-4 | 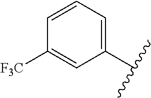 [SMILES: *c1cccc(C(F)(F)F)c1] | 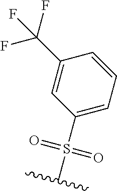 [SMILES: *S(=O)(=O)c1cccc(C(F)(F)F)c1] | 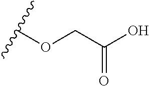 [SMILES: *OCC(=O)O] |
| III-5 | 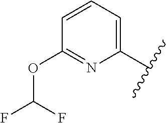 [SMILES: *c1cccc(OC(F)F)n1] | 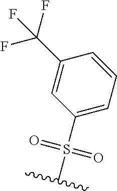 [SMILES: *S(=O)(=O)c1cccc(C(F)(F)F)c1] | 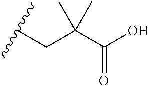 [SMILES: *CC(C)(C)C(=O)O] |
| III-6 | 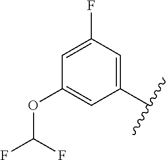 [SMILES: *c1cc(F)cc(OC(F)F)c1] | 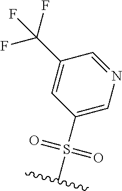 [SMILES: *S(=O)(=O)c1cncc(C(F)(F)F)c1] | 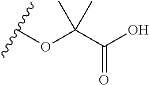 [SMILES: *OC(C)(C)C(=O)O] |
| III-7 | 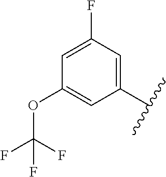 [SMILES: *c1cc(F)cc(OC(F)(F)F)c1] | 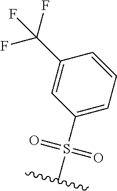 [SMILES: *S(=O)(=O)c1cccc(C(F)(F)F)c1] | 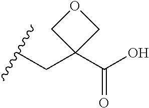 [SMILES: *CC1(C(=O)O)COC1] |
| III-8 | 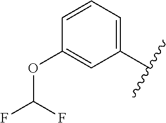 [SMILES: *c1cccc(OC(F)F)c1] | 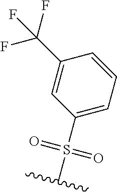 [SMILES: *S(=O)(=O)c1cccc(C(F)(F)F)c1] | 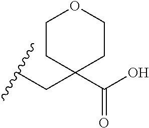 [SMILES: *CC1(C(=O)O)CCOCC1] |
| III-9 | 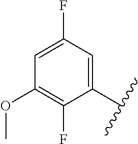 [SMILES: *c1cc(F)cc(OC)c1F] | 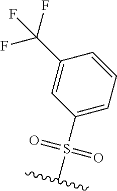 [SMILES: *S(=O)(=O)c1cccc(C(F)(F)F)c1] | 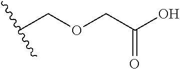 [SMILES: *COCC(=O)O] |
| III-10 | 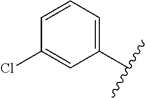 [SMILES: *c1cccc(Cl)c1] | 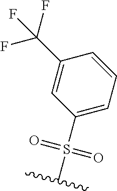 [SMILES: *S(=O)(=O)c1cccc(C(F)(F)F)c1] | 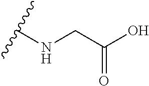 [SMILES: *NCC(=O)O] |
| III-11 | 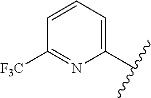 [SMILES: *c1cccc(C(F)(F)F)n1] | 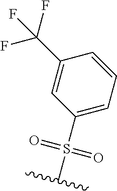 [SMILES: *S(=O)(=O)c1cccc(C(F)(F)F)c1] | 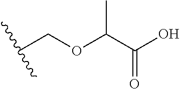 [SMILES: *COC(C)C(=O)O] |

TABLE 4

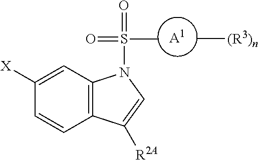

| No. | X | 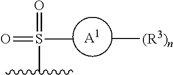 | R^{2A} |
|---|---|---|---|
| IV-1 | 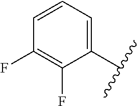 [SMILES: *c1cccc(F)c1F] | 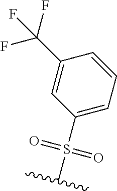 [SMILES: *S(=O)(=O)c1cccc(C(F)(F)F)c1] | H |
| IV-2 | 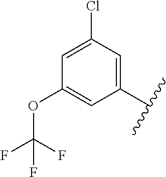 [SMILES: *c1cc(Cl)cc(OC(F)(F)F)c1] | 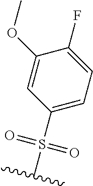 [SMILES: *S(=O)(=O)c1ccc(F)c(OC)c1] | 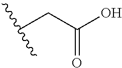 [SMILES: *CC(=O)O] |
| IV-3 | 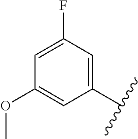 [SMILES: *c1cc(F)cc(OC)c1] | 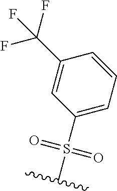 [SMILES: *S(=O)(=O)c1cccc(C(F)(F)F)c1] | 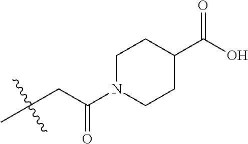 [SMILES: *CC(=O)N1CCC(C(=O)O)CC1] |
| IV-4 | 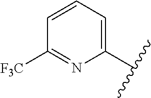 [SMILES: *c1cccc(C(F)(F)F)n1] | 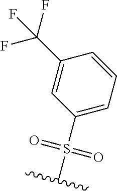 [SMILES: *S(=O)(=O)c1cccc(C(F)(F)F)c1] | 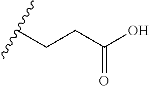 [SMILES: *CCC(=O)O] |
| IV-5 | 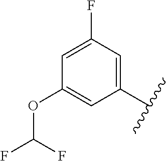 [SMILES: *c1cc(F)cc(OC(F)F)c1] | 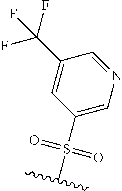 [SMILES: *S(=O)(=O)c1cncc(C(F)(F)F)c1] | 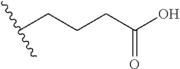 [SMILES: *CCCC(=O)O] |
| IV-6 | 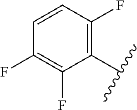 [SMILES: *c1c(F)ccc(F)c1F] | 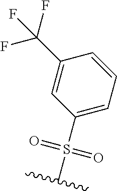 [SMILES: *S(=O)(=O)c1cccc(C(F)(F)F)c1] | 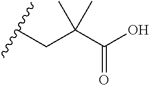 [SMILES: *CC(C)(C)C(=O)O] |
| IV-7 | 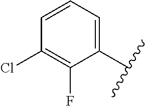 [SMILES: *c1cccc(Cl)c1F] | 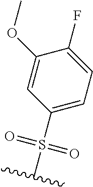 [SMILES: *S(=O)(=O)c1ccc(F)c(OC)c1] | 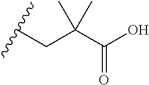 [SMILES: *CC(C)(C)C(=O)O] |
| IV-8 | 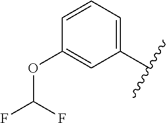 [SMILES: *c1cccc(OC(F)F)c1] | 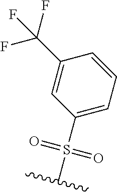 [SMILES: *S(=O)(=O)c1cccc(C(F)(F)F)c1] | 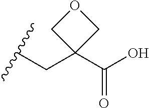 [SMILES: *CC1(C(=O)O)COC1] |
| IV-9 | 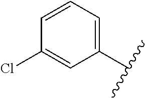 [SMILES: *c1cccc(Cl)c1] | 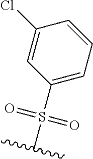 [SMILES: *S(=O)(=O)c1cccc(Cl)c1] | 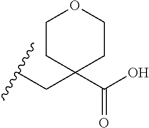 [SMILES: *CC1(C(=O)O)CCOCC1] |
| IV-10 | 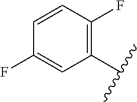 [SMILES: *c1cc(F)ccc1F] | 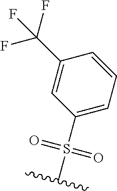 [SMILES: *S(=O)(=O)c1cccc(C(F)(F)F)c1] | 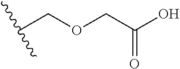 [SMILES: *COCC(=O)O] |
| IV-11 | 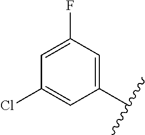 [SMILES: *c1cc(F)cc(Cl)c1] | 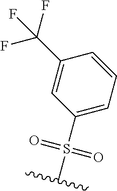 [SMILES: *S(=O)(=O)c1cccc(C(F)(F)F)c1] | 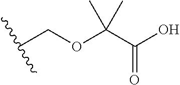 [SMILES: *COC(C)(C)C(=O)O] |
| IV-12 | 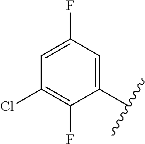 [SMILES: *c1cc(F)cc(Cl)c1F] | 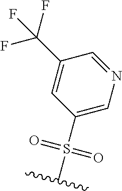 [SMILES: *S(=O)(=O)c1cncc(C(F)(F)F)c1] | 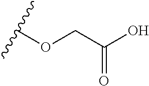 [SMILES: *OCC(=O)O] |

TABLE 5

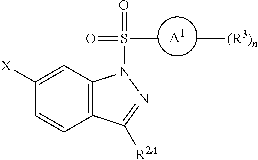

| No. | X | 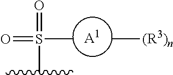 | R^{2A} |
|---|---|---|---|
| V-1 | 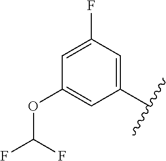 [SMILES: *c1cc(F)cc(OC(F)F)c1] | 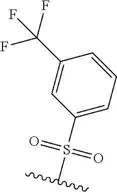 [SMILES: *S(=O)(=O)c1cccc(C(F)(F)F)c1] | H |
| V-2 | 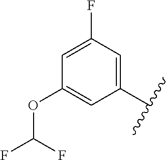 [SMILES: *c1cc(F)cc(OC(F)F)c1] | 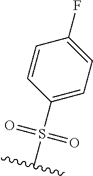 [SMILES: *S(=O)(=O)c1ccc(F)cc1] | H |
| V-3 | 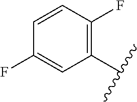 [SMILES: *c1cc(F)ccc1F] | 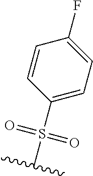 [SMILES: *S(=O)(=O)c1ccc(F)cc1] | H |
| V-4 | 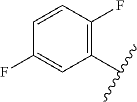 [SMILES: *c1cc(F)ccc1F] | 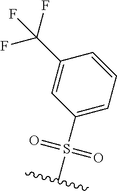 [SMILES: *S(=O)(=O)c1cccc(C(F)(F)F)c1] | 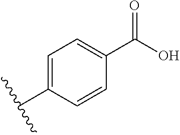 [SMILES: *c1ccc(C(=O)O)cc1] |
| V-5 | 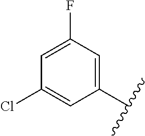 [SMILES: *c1cc(F)cc(Cl)c1] | 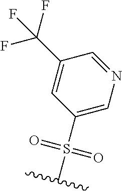 [SMILES: *S(=O)(=O)c1cncc(C(F)(F)F)c1] | 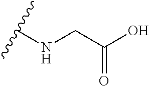 [SMILES: *NCC(=O)O] |
| V-6 | 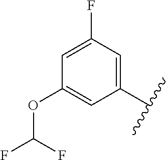 [SMILES: *c1cc(F)cc(OC(F)F)c1] | 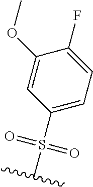 [SMILES: *S(=O)(=O)c1ccc(F)c(OC)c1] | 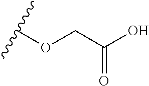 [SMILES: *OCC(=O)O] |
| V-7 | 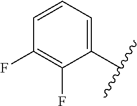 [SMILES: *c1cccc(F)c1F] | 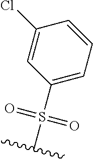 [SMILES: *S(=O)(=O)c1cccc(Cl)c1] | 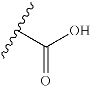 [SMILES: *C(=O)O] |
| V-8 | 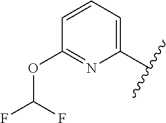 [SMILES: *c1cccc(OC(F)F)n1] | 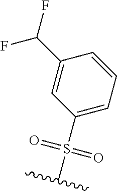 [SMILES: *S(=O)(=O)c1cccc(C(F)F)c1] | 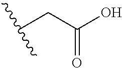 [SMILES: *CC(=O)O] |
| V-9 | 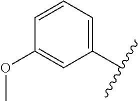 [SMILES: *c1cccc(OC)c1] | 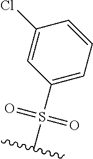 [SMILES: *S(=O)(=O)c1cccc(Cl)c1] | 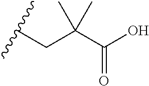 [SMILES: *CC(C)(C)C(=O)O] |
| V-10 | 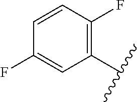 [SMILES: *c1cc(F)ccc1F] | 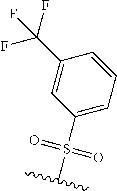 [SMILES: *S(=O)(=O)c1cccc(C(F)(F)F)c1] | 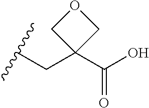 [SMILES: *CC1(C(=O)O)COC1] |

TABLE 6

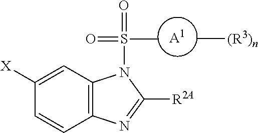

| No. | X | 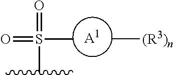 | R^{2A} |
|---|---|---|---|
| VI-1 | 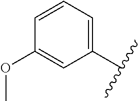 [SMILES: *c1cccc(OC)c1] | 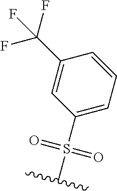 [SMILES: *S(=O)(=O)c1cccc(C(F)(F)F)c1] | H |
| VI-2 | 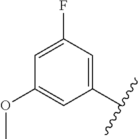 [SMILES: *c1cc(F)cc(OC)c1] | 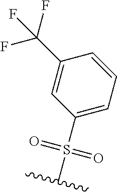 [SMILES: *S(=O)(=O)c1cccc(C(F)(F)F)c1] | 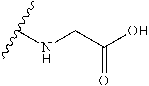 [SMILES: *NCC(=O)O] |
| VI-3 | 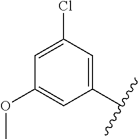 [SMILES: *c1cc(Cl)cc(OC)c1] | 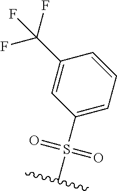 [SMILES: *S(=O)(=O)c1cccc(C(F)(F)F)c1] | 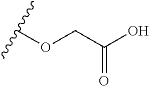 [SMILES: *OCC(=O)O] |
| VI-4 | 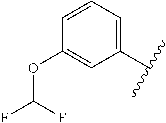 [SMILES: *c1cccc(OC(F)F)c1] | 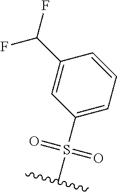 [SMILES: *S(=O)(=O)c1cccc(C(F)F)c1] | 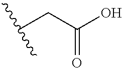 [SMILES: *CC(=O)O] |
| VI-5 | 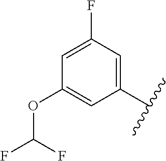 [SMILES: *c1cc(F)cc(OC(F)F)c1] | 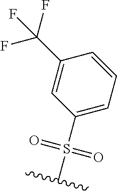 [SMILES: *S(=O)(=O)c1cccc(C(F)(F)F)c1] | 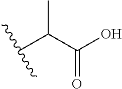 [SMILES: *C(C)C(=O)O] |
| VI-6 | 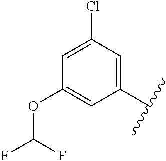 [SMILES: *c1cc(Cl)cc(OC(F)F)c1] | 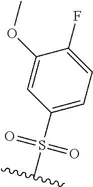 [SMILES: *S(=O)(=O)c1ccc(F)c(OC)c1] | 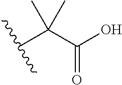 [SMILES: *C(C)(C)C(=O)O] |
| VI-7 | 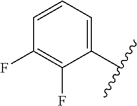 [SMILES: *c1cccc(F)c1F] | 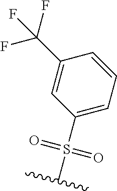 [SMILES: *S(=O)(=O)c1cccc(C(F)(F)F)c1] | 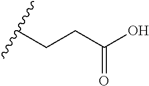 [SMILES: *CCC(=O)O] |
| VI-8 | 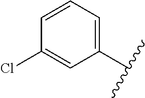 [SMILES: *c1cccc(Cl)c1] | 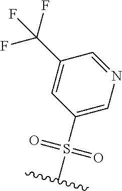 [SMILES: *S(=O)(=O)c1cncc(C(F)(F)F)c1] | 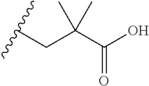 [SMILES: *CC(C)(C)C(=O)O] |
| VI-9 | 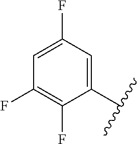 [SMILES: *c1cc(F)cc(F)c1F] | 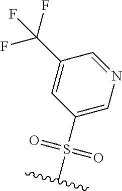 [SMILES: *S(=O)(=O)c1cncc(C(F)(F)F)c1] | 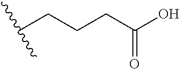 [SMILES: *CCCC(=O)O] |
| VI-10 | 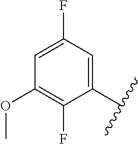 [SMILES: *c1cc(F)cc(OC)c1F] | 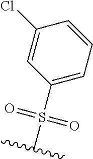 [SMILES: *S(=O)(=O)c1cccc(Cl)c1] | 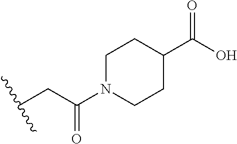 [SMILES: *CC(=O)N1CCC(C(=O)O)CC1] |
| VI-11 | 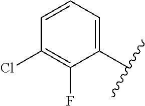 [SMILES: *c1cccc(Cl)c1F] | 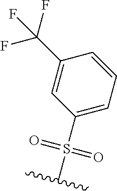 [SMILES: *S(=O)(=O)c1cccc(C(F)(F)F)c1] | 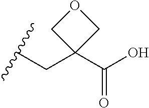 [SMILES: *CC1(C(=O)O)COC1] |
| VI-12 | 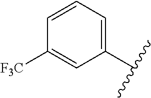 [SMILES: *c1cccc(C(F)(F)F)c1] | 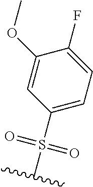 [SMILES: *S(=O)(=O)c1ccc(F)c(OC)c1] | 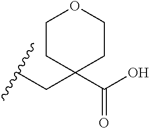 [SMILES: *CC1(C(=O)O)CCOCC1] |

TABLE 7

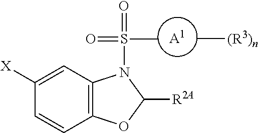

| No. | X | 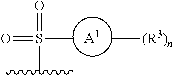 | R^{2A} |
|---|---|---|---|
| VII-1 | 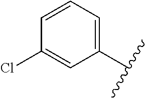 [SMILES: *c1cccc(Cl)c1] | 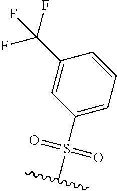 [SMILES: *S(=O)(=O)c1cccc(C(F)(F)F)c1] | 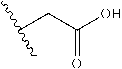 [SMILES: *CC(=O)O] |
| VII-2 | 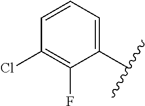 [SMILES: *c1cccc(Cl)c1F] | 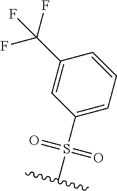 [SMILES: *S(=O)(=O)c1cccc(C(F)(F)F)c1] | 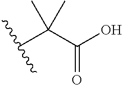 [SMILES: *C(C)(C)C(=O)O] |
| VII-3 | 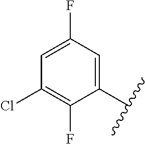 [SMILES: *c1cc(F)cc(Cl)c1F] | 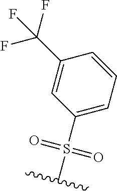 [SMILES: *S(=O)(=O)c1cccc(C(F)(F)F)c1] | 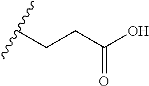 [SMILES: *CCC(=O)O] |
| VII-4 | 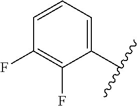 [SMILES: *c1cccc(F)c1F] | 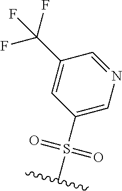 [SMILES: *S(=O)(=O)c1cncc(C(F)(F)F)c1] | 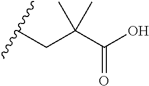 [SMILES: *CC(C)(C)C(=O)O] |
| VII-5 | 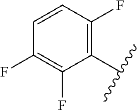 [SMILES: *c1c(F)ccc(F)c1F] | 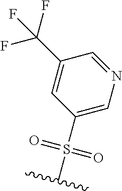 [SMILES: *S(=O)(=O)c1cncc(C(F)(F)F)c1] | 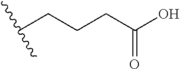 [SMILES: *CCCC(=O)O] |
| VII-6 | 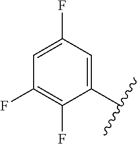 [SMILES: *c1cc(F)cc(F)c1F] | 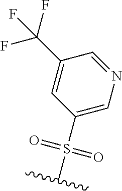 [SMILES: *S(=O)(=O)c1cncc(C(F)(F)F)c1] | 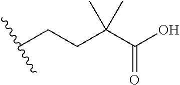 [SMILES: *CCC(C)(C)C(=O)O] |
| VII-7 | 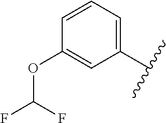 [SMILES: *c1cccc(OC(F)F)c1] | 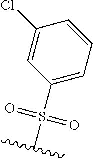 [SMILES: *S(=O)(=O)c1cccc(Cl)c1] | 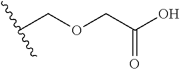 [SMILES: *COCC(=O)O] |
| VII-8 | 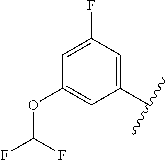 [SMILES: *c1cc(F)cc(OC(F)F)c1] | 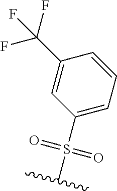 [SMILES: *S(=O)(=O)c1cccc(C(F)(F)F)c1] | 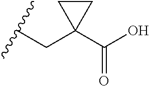 [SMILES: *CC1(C(=O)O)CC1] |
| VII-9 | 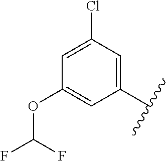 [SMILES: *c1cc(Cl)cc(OC(F)F)c1] | 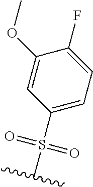 [SMILES: *S(=O)(=O)c1ccc(F)c(OC)c1] | 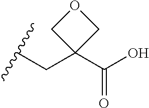 [SMILES: *CC1(C(=O)O)COC1] |
| VII-10 | 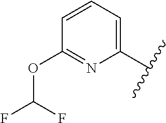 [SMILES: *c1cccc(OC(F)F)n1] | 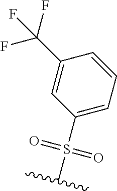 [SMILES: *S(=O)(=O)c1cccc(C(F)(F)F)c1] | H |
| VII-11 | 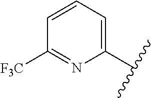 [SMILES: *c1cccc(C(F)(F)F)n1] | 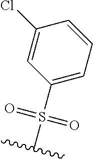 [SMILES: *S(=O)(=O)c1cccc(Cl)c1] | 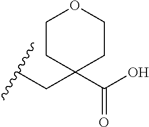 [SMILES: *CC1(C(=O)O)CCOCC1] |
| VII-12 | 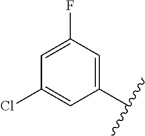 [SMILES: *c1cc(F)cc(Cl)c1] | 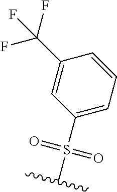 [SMILES: *S(=O)(=O)c1cccc(C(F)(F)F)c1] | 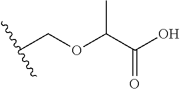 [SMILES: *COC(C)C(=O)O] |

TABLE 8

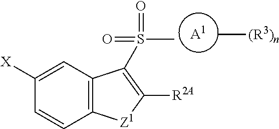

| No. | X | 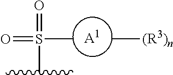 | R^{2A} | Z¹ |
|---|---|---|---|---|
| VIII-1 | 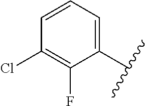 [SMILES: *c1cccc(Cl)c1F] | 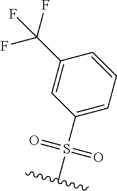 [SMILES: *S(=O)(=O)c1cccc(C(F)(F)F)c1] | H | O |
| VIII-2 | 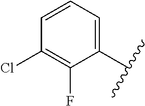 [SMILES: *c1cccc(Cl)c1F] | 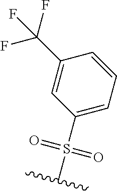 [SMILES: *S(=O)(=O)c1cccc(C(F)(F)F)c1] | H | NH |
| VIII-3 | 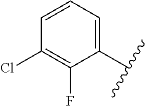 [SMILES: *c1cccc(Cl)c1F] | 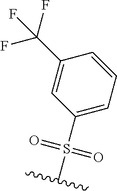 [SMILES: *S(=O)(=O)c1cccc(C(F)(F)F)c1] | H | NMe |
| VIII-4 | 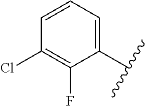 [SMILES: *c1cccc(Cl)c1F] | 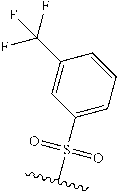 [SMILES: *S(=O)(=O)c1cccc(C(F)(F)F)c1] | 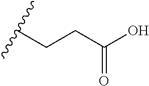 [SMILES: *CCC(=O)O] | O |
| VIII-5 | 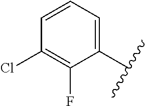 [SMILES: *c1cccc(Cl)c1F] | 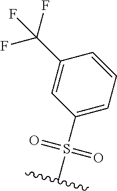 [SMILES: *S(=O)(=O)c1cccc(C(F)(F)F)c1] | 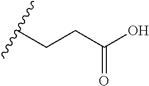 [SMILES: *CCC(=O)O] | NH |
| VIII-6 | 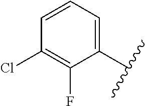 [SMILES: *c1cccc(Cl)c1F] | 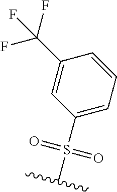 [SMILES: *S(=O)(=O)c1cccc(C(F)(F)F)c1] | 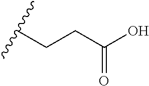 [SMILES: *CCC(=O)O] | NMe |
| VIII-7 | 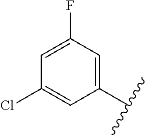 [SMILES: *c1cc(F)cc(Cl)c1] | 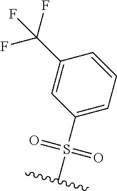 [SMILES: *S(=O)(=O)c1cccc(C(F)(F)F)c1] | 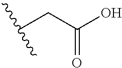 [SMILES: *CC(=O)O] | O |
| VIII-8 | 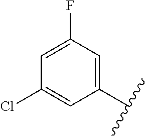 [SMILES: *c1cc(F)cc(Cl)c1] | 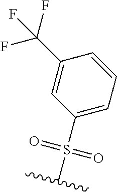 [SMILES: *S(=O)(=O)c1cccc(C(F)(F)F)c1] | 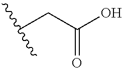 [SMILES: *CC(=O)O] | NH |
| VIII-9 | 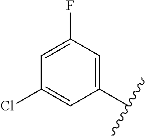 [SMILES: *c1cc(F)cc(Cl)c1] | 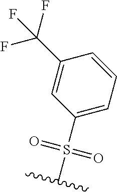 [SMILES: *S(=O)(=O)c1cccc(C(F)(F)F)c1] | 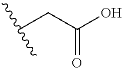 [SMILES: *CC(=O)O] | NMe |
| VIII-10 | 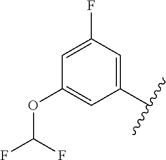 [SMILES: *c1cc(F)cc(OC(F)F)c1] | 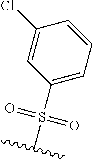 [SMILES: *S(=O)(=O)c1cccc(Cl)c1] | 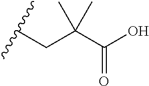 [SMILES: *CC(C)(C)C(=O)O] | O |
| VIII-11 | 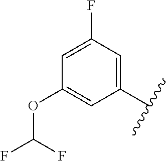 [SMILES: *c1cc(F)cc(OC(F)F)c1] | 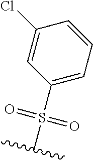 [SMILES: *S(=O)(=O)c1cccc(Cl)c1] | 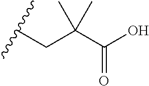 [SMILES: *CC(C)(C)C(=O)O] | NH |
| VIII-12 | 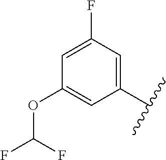 [SMILES: *c1cc(F)cc(OC(F)F)c1] | 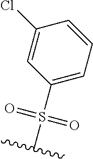 [SMILES: *S(=O)(=O)c1cccc(Cl)c1] | 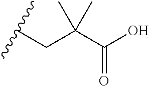 [SMILES: *CC(C)(C)C(=O)O] | NMe |
| VIII-13 | 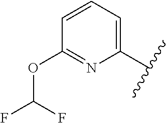 [SMILES: *c1cccc(OC(F)F)n1] | 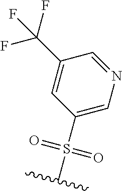 [SMILES: *S(=O)(=O)c1cncc(C(F)(F)F)c1] | 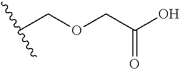 [SMILES: *COCC(=O)O] | O |
| VIII-14 | 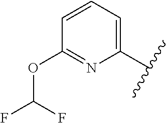 [SMILES: *c1cccc(OC(F)F)n1] | 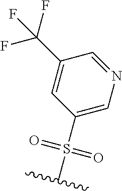 [SMILES: *S(=O)(=O)c1cncc(C(F)(F)F)c1] | 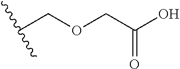 [SMILES: *COCC(=O)O] | NH |
| VIII-15 | 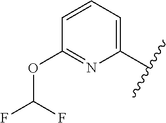 [SMILES: *c1cccc(OC(F)F)n1] | 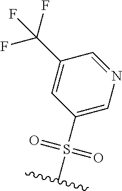 [SMILES: *S(=O)(=O)c1cncc(C(F)(F)F)c1] | 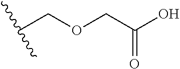 [SMILES: *COCC(=O)O] | NMe |

TABLE 9

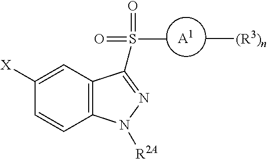

| No. | X | 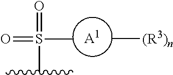 | R^{2A} |
|---|---|---|---|
| IX-1 | 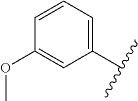 [SMILES: *c1cccc(OC)c1] | 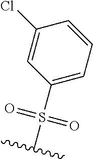 [SMILES: *S(=O)(=O)c1cccc(Cl)c1] | 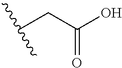 [SMILES: *CC(=O)O] |
| IX-2 | 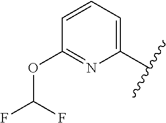 [SMILES: *c1cccc(OC(F)F)n1] | 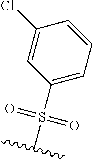 [SMILES: *S(=O)(=O)c1cccc(Cl)c1] | 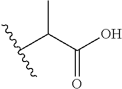 [SMILES: *C(C)C(=O)O] |
| IX-3 | 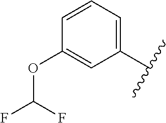 [SMILES: *c1cccc(OC(F)F)c1] | 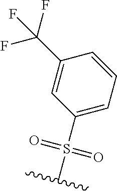 [SMILES: *S(=O)(=O)c1cccc(C(F)(F)F)c1] | 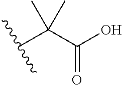 [SMILES: *C(C)(C)C(=O)O] |
| IX-4 | 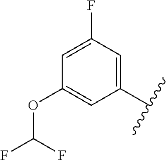 [SMILES: *c1cc(F)cc(OC(F)F)c1] | 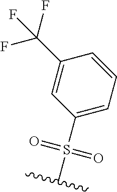 [SMILES: *S(=O)(=O)c1cccc(C(F)(F)F)c1] | 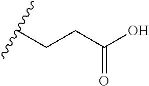 [SMILES: *CCC(=O)O] |
| IX-5 | 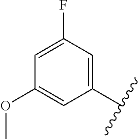 [SMILES: *c1cc(F)cc(OC)c1] | 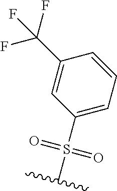 [SMILES: *S(=O)(=O)c1cccc(C(F)(F)F)c1] | 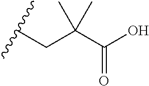 [SMILES: *CC(C)(C)C(=O)O] |
| IX-6 | 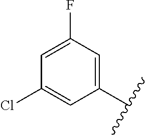 [SMILES: *c1cc(F)cc(Cl)c1] | 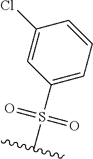 [SMILES: *S(=O)(=O)c1cccc(Cl)c1] | 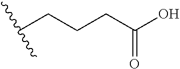 [SMILES: *CCCC(=O)O] |
| IX-7 | 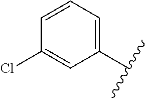 [SMILES: *c1cccc(Cl)c1] | 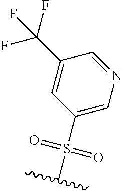 [SMILES: *S(=O)(=O)c1cncc(C(F)(F)F)c1] | 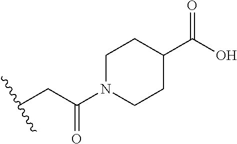 [SMILES: *CC(=O)N1CCC(C(=O)O)CC1] |
| IX-8 | 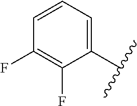 [SMILES: *c1cccc(F)c1F] | 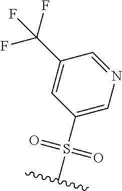 [SMILES: *S(=O)(=O)c1cncc(C(F)(F)F)c1] | H |
| IX-9 | 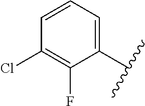 [SMILES: *c1cccc(Cl)c1F] | 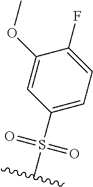 [SMILES: *S(=O)(=O)c1ccc(F)c(OC)c1] | 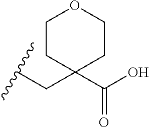 [SMILES: *CC1(C(=O)O)CCOCC1] |
| IX-10 | 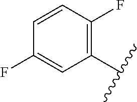 [SMILES: *c1cc(F)ccc1F] | 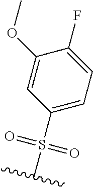 [SMILES: *S(=O)(=O)c1ccc(F)c(OC)c1] | 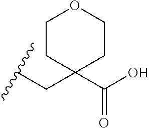 [SMILES: *CC1(C(=O)O)CCOCC1] |

In certain other embodiments, the compound is one of the compounds listed in Table 10 herein or a pharmaceutically acceptable salt thereof. In certain embodiments, the compound is one of the compounds listed in Table 10 herein.

Methods for preparing compounds described herein are illustrated in the following synthetic Schemes. The Schemes are given for the purpose of illustrating the invention, and are not intended to limit the scope or spirit of the invention. Starting materials shown in the Schemes can be obtained from commercial sources or be prepared based on procedures described in the literature.

The synthetic route illustrated in Scheme 1 is a general method for preparing substituted N-sulfonyl indole compound C and indolinyl sulfonamide compound D. Reaction of bromoindole A with a sulfonyl chloride provides N-sulfonyl indole B. Reaction of compound B with an aryl or heteroaryl boronate compound under cross-coupling conditions (for instance, with Pd or another transition metal) affords N-sulfonyl indole C. Reduction of compound C, such as using a hydride or metal-mediated hydrogenation (e.g., NaBH₃CN or Pd/C and H₂, respectively) provides indolinyl sulfonamide D. The order in which these reactions are conducted can be varied, such that indolinyl sulfonamide D is prepared from bromoindole A, for example, by reduction, then sulfonylation, and then metal-catalyzed cross-coupling.

The reaction procedures in Scheme 1 are contemplated to be amenable to preparing a wide variety of N-sulfonyl indole and indolinyl sulfonamide compounds having different substituents at the R, R^{I}, R^{II}, and R^{III} positions. For example, numerous substituted bromoindoles are known in the literature and/or are commercially available or readily prepared using the broad range of procedures developed for the synthesis of indoles. Furthermore, if a functional group on a molecule would not be amenable to a reaction condition described in Scheme 1, it is contemplated that the functional group can first be protected using standard protecting group chemistry and strategies, and then the protecting group is removed after completing the desired synthetic transformation. See, for example, Greene, T. W.; Wuts, P. G. M. Protective Groups in Organic Synthesis, 2^{d} ed.; Wiley: New York, 1991, for further description of protecting chemistry and strategies. In certain other embodiments, a functional group in substituent R through R^{III} in N-sulfonyl indole C or indolinyl sulfonamide D can be converted to another functional group using standard functional group manipulation procedures known in the art. See, for example, “Comprehensive Organic Synthesis” (B. M. Trost & I. Fleming, eds., 1991-1992

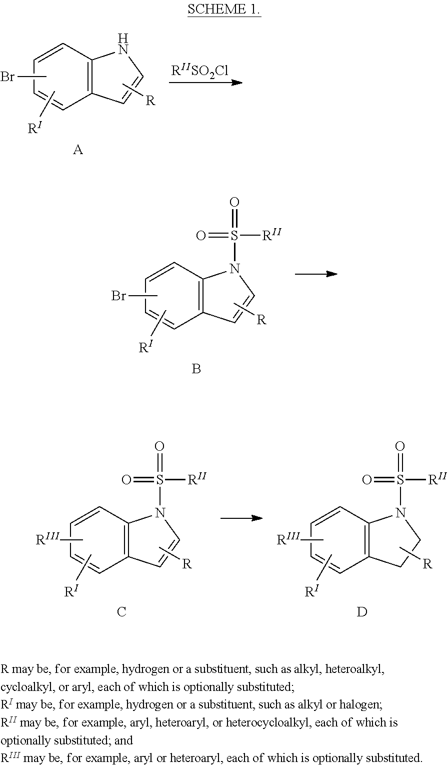

Scheme 2 illustrates a general method for preparing substituted N-sulfonyl indazole C. Reaction of bromoindazole A with a sulfonyl chloride provides N-sulfonyl indazole B. Reaction of compound B with an aryl or heteroaryl boronate compound under cross-coupling conditions (for instance, with Pd or other transition metal) affords N-sulfonyl indazole C.

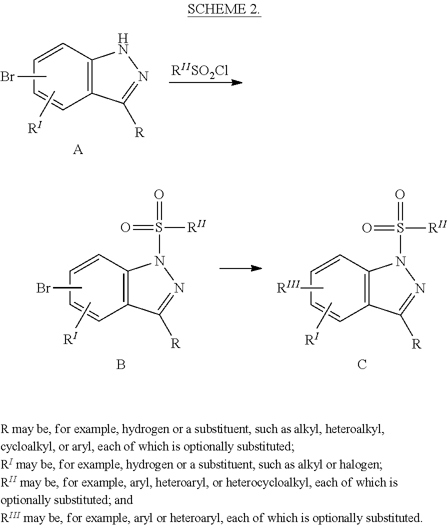

Scheme 3 illustrates a general method for preparing substituted N-sulfonyl benzimidazole D. Condensation of bromo-1,2-phenylenediamine A with a carboxylic acid or carboxylic acid derivative yields bromobenzimidazole B. Reaction of bromobenzimidazole B with a sulfonyl chloride provides N-sulfonyl benzimidazole C. Reaction of compound C with an aryl or heteroaryl boronate compound under cross-coupling conditions (for instance, with Pd or other transition metal) affords N-sulfonyl benzimidazole D.

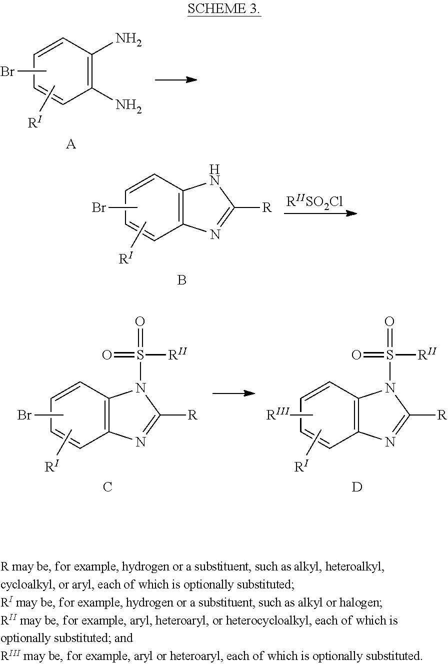

Scheme 4 illustrates a general method for preparing substituted N-sulfonyl 2,3-dihydrobenzo[d]oxazole D. Reaction of aminophenol A with a sulfonyl chloride provides sulfonamide B. Acid-catalyzed condensation (for instance, with p-toluenesulfonic acid; see, for example, US 2007/0191603) of compound B with a dimethyl acetal or ketal yields N-sulfonyl 2,3-dihydrobenzo[d]oxazole C. Reaction of compound C with an aryl or heteroaryl boronate compound under cross-coupling conditions (for instance, with Pd or other transition metal) affords N-sulfonyl 2,3-dihydrobenzo[d]oxazole D.

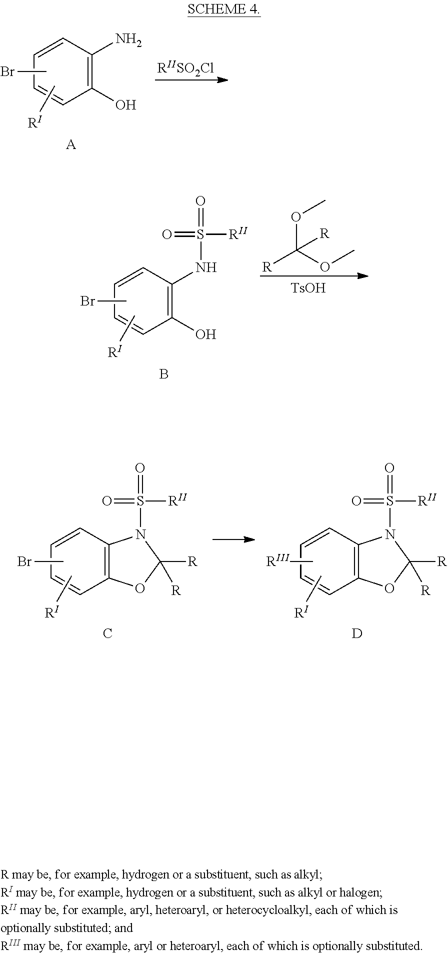

Scheme 5 illustrates a general procedure for preparing substituted 3-sulfonyl benzofuran E. Acetoxy benzyl chloride A is converted to sulfone B by addition of a sodium sulfinate. The acetoxy group of sulfone B is removed (for instance, with sodium bicarbonate) and the resulting phenol is acylated with a carboxylic acid (or derivative) to provide acyloxy sulfone C. Cyclization of sulfone C occurs upon treatment with base, and then acid-mediated dehydration affords benzofuran D (see, for example, Y. Liu et al., Tetrahedron Lett. 2011, Vol. 52, No. 23, pages 2935-2939). Reaction of benzofuran D with an aryl or heteroaryl boronate compound under cross-coupling conditions (for instance, with Pd or other transition metal) provides 3-sulfonyl benzofuran E.

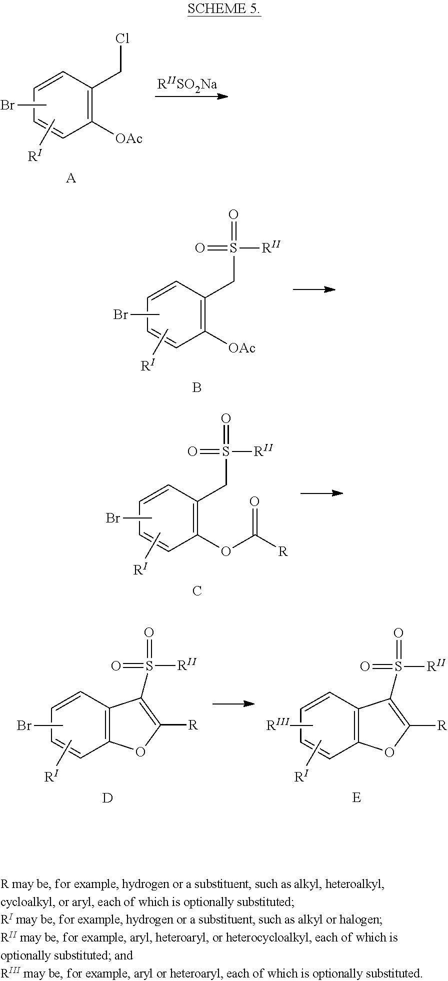

Scheme 6 illustrates a general procedure for preparing substituted 3-sulfonyl indoles C and D. Reaction of bromoindole A with a sulfonyl chloride under catalytic conditions (e.g., using copper(I) iodide; see, for example, Rahman, M. J Sulfur Chem. 2013, Vol. 34, No. 4, pages 342-346) provides 3-sulfonyl indole B. Reaction of compound B with an aryl or heteroaryl boronate compound under cross-coupling conditions (for instance, with Pd or other transition metal) affords 3-sulfonyl indole C. Treatment of compound C with an alkylating agent yields N-alkylated indole D.

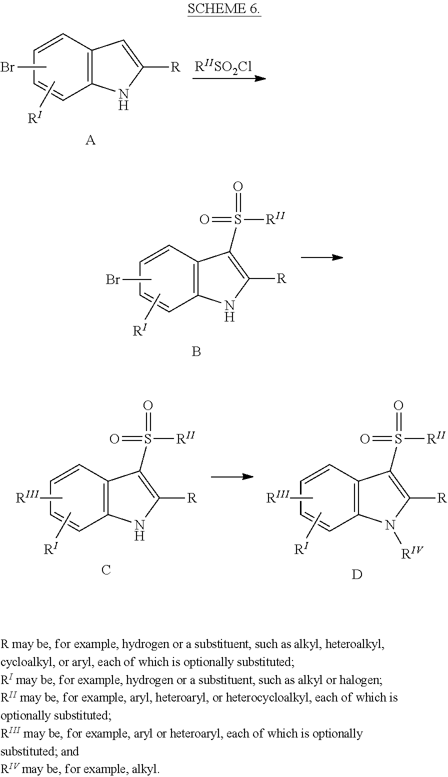

Scheme 7 illustrates a general procedure for preparing substituted 3-sulfonyl indazoles C and D. Reaction of iodobromoindazole A with a sodium sulfinate under catalytic conditions (e.g., using copper(I) iodide; see, for example, US 2004/0167122) provides 3-sulfonyl bromoindazole B. Reaction of compound B with an aryl or heteroaryl boronate compound under cross-coupling conditions (for instance, with Pd or other transition metals) affords 3-sulfonyl indazole C. Treatment of compound C with an alkylating agent yields N-alkylated indazole D. Alternatively, reaction of compound C with an aryl boronate compound under cross-coupling conditions (for instance, with copper or other transition metals) affords N-aryl indazole D.

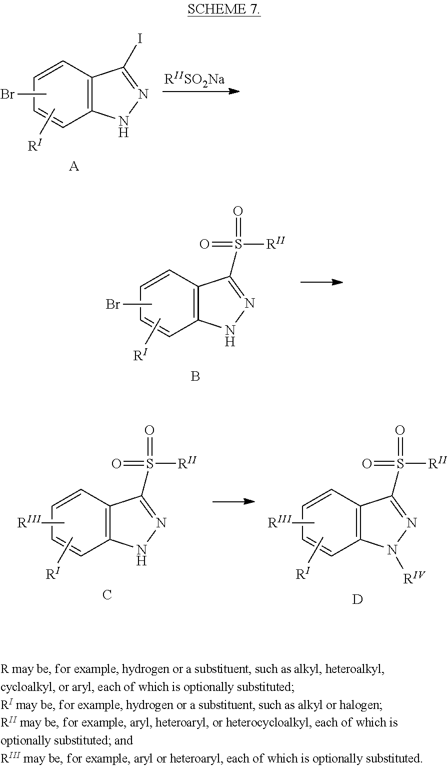

### II. Therapeutic Applications of Aryl Indolinyl Sulfonamide, Aryl Indolyl Sulfone, and Related Compounds

It is contemplated that the aryl indolinyl sulfonamide, aryl indolyl sulfone, and related compounds described herein, such as a compound of Formula I, I-A, I-A′, I-B, I-B′, I-C, I-D, II, II-A, II-B, or other compounds in Section I, provide therapeutic benefits to subjects suffering from a cancer, bacterial infection, fungal infection, or immune deficiency disorder. Accordingly, one aspect of the invention provides a method of treating a disorder selected from the group consisting of cancer, bacterial infection, fungal infection, and immune deficiency disorder. The method comprises administering a therapeutically effective amount of an aryl indolinyl sulfonamide, aryl indolyl sulfone, or related compound described herein, such as a compound of Formula I, I-A, I-A′, I-B, I-B′, I-C, I-D, II, II-A, II-B, or other compounds in Section I, to a subject in need thereof to treat the disorder. In certain embodiments, the particular compound of Formula I, I-A, I-A′, I-B, I-B′, I-C, I-D, II, II-A, or II-B is a compound defined by one of the embodiments described above.

In certain embodiments, the disorder is cancer. In certain embodiments, the cancer is a solid tumor or leukemia. In certain other embodiments, the cancer is colon cancer, pancreatic cancer, breast cancer, ovarian cancer, prostate cancer, squamous cell carcinoma, basal cell carcinoma, adenocarcinoma, sweat gland carcinoma, sebaceous gland carcinoma, lung cancer, leukemia, bladder cancer, stomach cancer, cervical cancer, testicular cancer, skin cancer, rectal cancer, thyroid cancer, kidney cancer, uterus cancer, esophagus cancer, liver cancer, an acoustic neuroma, oligodendroglioma, meningioma, neuroblastoma, or retinoblastoma. In certain other embodiments, the cancer is small cell lung cancer, non-small cell lung cancer, melanoma, cancer of the central nervous system tissue, brain cancer, Hodgkin's lymphoma, non-Hodgkin's lymphoma, cutaneous T-Cell lymphoma, cutaneous B-Cell lymphoma, or diffuse large B-Cell lymphoma. In certain other embodiments, the cancer is breast cancer, colon cancer, small-cell lung cancer, non-small cell lung cancer, prostate cancer, renal cancer, ovarian cancer, leukemia, melanoma, or cancer of the central nervous system tissue. In certain other embodiments, the cancer is colon cancer, small-cell lung cancer, non-small cell lung cancer, renal cancer, ovarian cancer, renal cancer, or melanoma.

Additional exemplary cancers include fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, epithelial carcinoma, glioma, astrocytoma, medulloblastoma, and hemangioblastoma.

In certain embodiments, the cancer is a neuroblastoma, meningioma, hemangiopericytoma, multiple brain metastase, glioblastoma multiforms, glioblastoma, brain stem glioma, poor prognosis malignant brain tumor, malignant glioma, anaplastic astrocytoma, anaplastic oligodendroglioma, neuroendocrine tumor, rectal adeno carcinoma, Dukes C & D colorectal cancer, unresectable colorectal carcinoma, metastatic hepatocellular carcinoma, Kaposi's sarcoma, karotype acute myeloblastic leukemia, Hodgkin's lymphoma, non-Hodgkin's lymphoma, cutaneous T-Cell lymphoma, cutaneous B-Cell lymphoma, diffuse large B-Cell lymphoma, low grade follicular lymphoma, metastatic melanoma, localized melanoma, malignant mesothelioma, malignant pleural effusion mesothelioma syndrome, peritoneal carcinoma, papillary serous carcinoma, gynecologic sarcoma, soft tissue sarcoma, scelroderma, cutaneous vasculitis, Langerhans cell histiocytosis, leiomyosarcoma, fibrodysplasia ossificans progressive, hormone refractory prostate cancer, resected high-risk soft tissue sarcoma, unrescectable hepatocellular carcinoma, Waidenstrom's macroglobulinemia, smoldering myeloma, indolent myeloma, fallopian tube cancer, androgen independent prostate cancer, androgen dependent stage IV non-metastatic prostate cancer, hormone-insensitive prostate cancer, chemotherapy-insensitive prostate cancer, papillary thyroid carcinoma, follicular thyroid carcinoma, medullary thyroid carcinoma, or leiomyoma.

In certain embodiments, the disorder is a bacterial infection. The bacterial infection can be characterized according to classifications known in the art. For example, in certain embodiments, the bacterial infection is a gram-positive bacterial infection, such as a gram-positive cocci bacterial infection or a gram-positive bacilli bacterial infection. In other embodiments, the bacterial infection is a gram-negative bacterial infection, such as a gram-negative cocci bacterial infection or a gram-negative bacilli bacterial infection. The bacterial infection can also be characterized according to whether it is caused by anaerobic or aerobic bacteria. Accordingly, in certain embodiments, the bacterial infection is an anaerobic bacterial infection. In certain other embodiments, the bacterial infection is an aerobic bacterial infection.

A variety of bacteria are contemplated to be susceptible to the tetrahydroquinoline compounds. Representative bacteria include Staphylococci species, e.g., S. aureus; Enterococci species, e.g., E. faecalis and E. faecium; Streptococci species, e.g., S. pyogenes and S. pneumoniae; Escherichia species, e.g., E. coli, including enterotoxigenic, enteropathogenic, enteroinvasive, enterohemorrhagic and enteroaggregative E. coli strains; Haemophilus species, e.g., H. influenza; and Moraxella species, e.g., M. catarrhalis. Other examples include Mycobacteria species, e.g., M. tuberculosis, M. avian-intracellulare, M. kansasii, M. bovis, M. africanum, M. genavense, M. leprae, M. xenopi, M. simiae, M. scrofulaceum, M. malmoense, M. celatum, M. abscessus, M. chelonae, M. szulgai, M. gordonae, M. haemophilum, M. fortuni and M. marinum; Corynebacteria species, e.g., C. diphtheriae; Vibrio species, e.g., V. cholerae; Campylobacter species, e.g., C. jejuni; Helicobacter species, e.g., H. pylori; Pseudomonas species, e.g., P. aeruginosa; Legionella species, e.g., L. pneumophila; Treponema species, e.g., T. pallidum; Borrelia species, e.g., B. burgdorferi; Listeria species, e.g., L. monocytogenes; Bacillus species, e.g., B. cereus; Bordatella species, e.g., B. pertussis; Clostridium species, e.g., C. perfringens, C. tetani, C. difficile and C. botulinum; Neisseria species, e.g., N. meningitidis and N. gonorrhoeae; Chlamydia species, e.g., C. psittaci, C. pneumoniae and C. trachomatis; Rickettsia species, e.g., R. rickettsii and R. prowazekii; Shigella species, e.g., S. sonnei; Salmonella species, e.g., S. typhimurium; Yersinia species, e.g., Y. enterocolitica and Y. pseudotuberculosis; Klebsiella species, e.g., K. pneumoniae; Mycoplasma species, e.g., M. pneumoniae; and Trypanosoma brucei. In certain embodiments, the compounds described herein are used to treat a subject suffering from a bacterial infection selected from the group consisting of S. aureus, E. faecalis, E. faecium, S. pyogenes, S. pneumonia, and P. aeruginosa.

The antibacterial activity of compounds described herein may be evaluated using assays known in the art, such as the microbroth dilution minimum inhibition concentration (MIC) assay, as further described in National Committee for Clinical Laboratory Standards. Performance Standards for Antimicrobial Susceptibility Testing; Fourteenth Informational Supplement. NCCLS document M100-S14 {ISBN 1-56238-516-X}. This assay may be used to determine the minimum concentration of a compound necessary to prevent visible bacterial growth in a solution. In general, the drug to be tested is serially diluted into wells, and aliquots of liquid bacterial culture are added. This mixture is incubated under appropriate conditions, and then tested for growth of the bacteria. Compounds with low or no antibiotic activity (a high MIC) will allow growth at high concentrations of compound, while compounds with high antibiotic activity will allow bacterial growth only at lower concentrations (a low MIC).

The assay uses stock bacterial culture conditions appropriate for the chosen strain of bacteria. Stock cultures from the permanent stock culture collection can be stored as frozen suspensions at −70° C. Cultures may be suspended in 10% skim milk (BD) prior to snap freezing in dry ice/ethanol and then placed in a −70° C. freezer. Cultures may be maintained on Tryptic Soy Agar containing 5% Sheep Blood at room temperature (20° C.), and each culture may be recovered from frozen form and transferred an additional time before MIC testing. Fresh plates are inoculated the day before testing, incubated overnight, and checked to confirm purity and identity.

The identity and purity of the cultures recovered from the stock culture can be confirmed to rule out the possibility of contamination. The identity of the strains may be confirmed by standard microbiological methods (See, e.g., Murray et al., Manual of Clinical Microbiology, Eighth Edition. ASM Press {ISBN 1-55581-255-4}). In general, cultures are streaked onto appropriate agar plates for visualization of purity, expected colony morphology, and hemolytic patterns. Gram stains can also be utilized. The identities are confirmed using a MicroScan WalkAway 40 SI Instrument (Dade Behring, West Sacramento, Calif.). This device utilizes an automated incubator, reader, and computer to assess for identification purposes the biochemical reactions carried out by each organism. The MicroScan WalkAway can also be used to determine a preliminary MIC, which may be confirmed using the method described below.

Frozen stock cultures may be used as the initial source of organisms for performing microbroth dilution minimum inhibition concentration (MIC) testing. Stock cultures are passed on their standard growth medium for at least 1 growth cycle (18-24 hours) prior to their use. Most bacteria may be prepared directly from agar plates in 10 mL aliquots of the appropriate broth medium. Bacterial cultures are adjusted to the opacity of a 0.5 McFarland Standard (optical density value of 0.28-0.33 on a Perkin-Elmer Lambda EZ150 Spectrophotometer, Wellesley, Mass., set at a wavelength of 600 nm). The adjusted cultures are then diluted 400 fold (0.25 mL inoculum+100 mL broth) in growth media to produce a starting suspension of approximately 5×105 colony forming units (CFU)/mL. Most bacterial strains may be tested in cation adjusted Mueller Hinton Broth (CAMHB).

Test compounds (“drugs”) are solubilized in a solvent suitable for the assay, such as DMSO. Drug stock solutions may be prepared on the day of testing. Microbroth dilution stock plates may be prepared in two dilution series, 64 to 0.06 μg drug/mL and 0.25 to 0.00025 μg drug/mL. For the high concentration series, 200 μL of stock solution (2 mg/mL) is added to duplicate rows of a 96-well microtiter plate. This is used as the first well in the dilution series. Serial two-fold decremental dilutions are made using a BioMek FX robot (Beckman Coulter Inc., Fullerton, Calif.) with 10 of the remaining 11 wells, each of which will contain 100 μL of the appropriate solvent/diluent. Row 12 contains solvent/diluent only and serves as the control. For the first well of the low concentration series, 200 μL of an 8 μg/mL stock are added to duplicate rows of a 96-well plate. Serial two-fold dilutions are made as described above.

Daughter 96-well plates may be spotted (3.2 μL/well) from the stock plates listed above using the BioMek FX robot and used immediately or frozen at −70° C. until use. Aerobic organisms are inoculated (100 μL volumes) into the thawed plates using the BioMek FX robot. The inoculated plates are placed in stacks and covered with an empty plate. These plates are then incubated for 16 to 24 hours in ambient atmosphere according to CLSI guidelines (National Committee for Clinical Laboratory Standards, Methods for Dilution, Antimicrobial Tests for Bacteria that Grow Aerobically; Approved Standard-Sixth Edition. NCCLS document M7-A6 {ISBN 1-56238-486-4}).

After inoculation and incubation, the degree of bacterial growth can be estimated visually with the aid of a Test Reading Mirror (Dynex Technologies 220 16) in a darkened room with a single light shining directly through the top of the microbroth tray. The MIC is the lowest concentration of drug that prevents macroscopically visible growth under the conditions of the test.

In certain embodiments, the disorder is a fungal infection. Exemplary fungi that may be treated include, for example, Acremonium, Absidia (e.g., Absidia corymbifera), Alternaria, Aspergillus (e.g., Aspergillus clavatus, Aspergillus flavus, Aspergillus fumigatus, Aspergillus nidulans, Aspergillus niger, Aspergillus terreus, and Aspergillus versicolor), Aureobasidium, Basidiobolus, Blastomyces (e.g., Blastomyces dermatitidis), Candida (e.g., Candida albicans, Candida glabrata, Candida guilliermondii, Candida kefyr, Candida krusei, Candida lusitaniae, Candida parapsilosis, Candida pseudotropicalis, Candida stellatoidea, Candida tropicalis, Candida utilis, Candida lipolytica, Candidafamata and Candida rugosa), Cephalosporium, Chaetomium, Chrysosporium, Cladosporium (e.g., Cladosporium carrionii and Cladosporium trichloides), Coccidioides (e.g., Coccidioides immitis), Conidiobolus, Coprinus, Corynespora, Cryptococcus (e.g., Cryptococcus neoformans), Curvularia, Cunninghamella (e.g., Cunninghamella elegans), Exophiala (e.g., Exophiala dermatitidis and Exophiala spinifera), Epidermophyton (e.g., Epidermophyton floccosum), Fonsecaea (e.g., Fonsecaea pedrosoi), Fusarium (e.g., Fusarium solani), Geotrichum (e.g., Geotrichum candiddum and Geotrichum clavatum), Hendersonula, Histoplasma, Leptosphaeria, Loboa, Madurella, Malassezia (e.g., Malassezia furfur), Microsporum (e.g., Microsporum canis and Microsporum gypseum), Mycocentrospora, Mucor, Neotestudina, Paecilomyces, Paracoccidioides (e.g., Paracoccidioides brasiliensis), Penicillium (e.g., Penicillium marneffei), Phialophora, Pneumocystis (e.g., Pneumocystis carinii), Pseudallescheria (e.g., Pseudallescheria boydii), Rhinosporidium, Rhizomucor, Rhizopus (e.g., Rhizopus microsporus var. rhizopodiformis and Rhizopus oryzae), Saccharomyces (e.g., Saccharomyces cerevisiae), Scopulariopsis, Sporothrix (e.g., Sporothrix schenckii), Trichophyton (e.g., Trichophyton mentagrophytes and Trichophyton rubrum), Trichosporon (e.g., Trichosporon asahii, Trichosporon beigelii and Trichosporon cutaneum), and Wangiella.

In certain embodiments, the disorder is an immune deficiency disorder. Exemplary immune deficiency disorders include, for example, a human immunodeficiency viral infection, a patient with a deficient immune system due to chemotherapy, or a patient recovering from surgery who has a deficient immune system.

In certain embodiments, the subject is a human.

Another aspect of the invention provides for the use of a compound described herein (such as a compound of Formula I, I-A, I-A′, I-B, I-B′, I-C, I-D, II, II-A, II-B, or other compounds in Section I) in the manufacture of a medicament. In certain embodiments, the medicament is for treating a disorder described herein, such as cancer.

Another aspect of the invention provides for the use of a compound described herein (such as a compound of Formula I, I-A, I-A′, I-B, I-B′, I-C, I-D, II, II-A, II-B, or other compounds in Section I) for treating a medical disorder, such as a medical disorder described herein (e.g., cancer).

### Promoting the Activity of RORγ

It is contemplated that aryl indolinyl sulfonamide, aryl indolyl sulfone, and related compounds described herein, such as a compound of Formula I, I-A, I-A′, I-B, I-B′, I-C, I-D, II, II-A, II-B, or other compounds in Section I, can promote the activity of RORγ. Accordingly, another aspect of the invention provides a method of promoting the activity of RORγ. The method comprises exposing a RORγ to an effective amount of an aryl indolinyl sulfonamide, aryl indolyl sulfone, or related compound described herein, such as a compound of Formula I, I-A, I-A′, I-B, I-B′, I-C, I-D, II, II-A, II-B, or other compounds in Section I, to promote RORγ activity. In certain embodiments, the particular compound of Formula I, I-A, I-A′, I-B, I-B′, I-C, I-D, II, II-A, or II-B is the compound defined by one of the embodiments described above. Promoting the activity of RORγ means to increase the activity of RORγ. In certain embodiments, exposing a RORγ to an effective amount of an aryl indolinyl sulfonamide, aryl indolyl sulfone, or related compound described herein (such as a compound of Formula I, I-A, I-A′, I-B, I-B′, I-C, I-D, II, II-A, II-B, or other compounds in Section I) results in an increase in RORγ activity of at least 5%, 10%, 20%, or 50% relative to the activity of RORγ under substantially the same conditions but without the presence of the aryl indolinyl sulfonamide, aryl indolyl sulfone, or related compound.

Ability of compounds to increase RORγ activity can be evaluated by testing the compounds in (i) a RORγ-Ligand Binding Domain (LBD) TR-FRET Assay, and/or (ii) a Gal4-RORγ Luciferase Reporter Assay in HEK-293T Cells. Exemplary assay procedures are described below.

### Exemplary Procedures for RORγ-Ligand Binding Domain TR-FRET Assay

HIS-tagged RORγ-LBD protein is expressed in SF9 cells using a baculovirus expression system. The lysate is diluted in assay buffer (50 mM Tris pH 7.0, 50 mM KCl, 1 mM EDTA, 0.1 mM DTT, 0.01% BSA) to obtain RORγ-LBD final concentration of −3 nM in a 384-well assay plate (need to titrate for each batch of protein).

A stock of biotinylated-LXXLL peptide from coactivator SRC1 (Biotin-CPSSHSSLTERHKILHRLLQEGSPS) is prepared in assay buffer and added to each well (200 nM final concentration). A solution of Europium tagged anti-HIS antibody (0.6 nM final concentration) and APC-conjugated streptavidin (30 nM final concentration) are also added to each well. RORγ antagonist ursolic acid is also included at a final concentration of 2 μM. Compounds are diluted in DMSO and further diluted in assay buffer with a final DMSO concentration at 1%. The highest concentration of test compound analyzed is 10 μM.

The final assay mixture is incubated overnight at 4° C. or 2 hours at room temperature, and the fluorescence signal may be measured on an Envision plate reader: (Excitation filter=340 nm; APC emission=665 nm; Europium emission=615 nm; dichroic mirror=D400/D630; delay time=100 μs, integration time=200 μs). 50% Effective concentration (EC₅₀) values for test compounds are calculated from the quotient of the fluorescence signal at 665 nm divided by the fluorescence signal at 615 nm. The quotient of the fluorescence signals in the absence of ursolic acid or test compound is set as 100. Max Response is defined as the upper plateau in the signal as determined by line-fit using a 4-parameter logistic model in PRISM (GraphPad).

### Exemplary Procedures for Gal4-RORγ Luciferase Reporter Assay in HEK-293T Cells Transfection of HEK-293 Cells

In the following protocol, HEK-293 cells are transfected with a construct comprising the Gal4 DNA binding domain fused to the ligand binding domain of RORγ (Gal4-RORγ-LBD) in a pcDNA3.1neo plasmid, and also with a reporter construct comprising pGL4.31 Gal4-luciferase (Promega). Control cells are prepared similarly using empty pcDNA3.1neo and pGL4.31 vectors.

Trans-IT reagent (Mirus, 60 μL) at room temperature is added drop wise to OptiMEM (Invitrogen, 1.5 ml). This reagent mixture is mixed by inversion then incubated for 5 to 30 minutes at room temperature. It then is added to a solution of both expression vectors (5 g each), mixed, and incubated at room temperature for about 20 minutes. HEK-293 cells are harvested from incubation flasks by removing the media, treating with TrypLE Express (Invitrogen), and incubating until the cells detached from the bottom of the flask (approximately 2-5 minutes). 10 Million cells are collected by centrifugation and re-suspended in 10 mL of Dulbecco's Modified Eagle Medium, High Glucose (DMEM, Invitrogen) containing 10% Fetal Bovine Serum and 100 IU each of penicillin and streptomycin. The re-suspended cells and the transfection mixture are added to a T75 flask, mixed and incubated overnight at 37° C. and 5% CO₂.

### Assay for RORγ Activity

The cells are harvested as described above, counted, and centrifuged to obtain the desired number of cells, then re-suspended in complete growth media at 0.75×10⁶ cells/mL. The RORγ antagonist, ursolic acid, is added to the cells at a final concentration of 2 μM. Cells are plated at 20 μL of cell suspension/well (10,000-15,000 cells/well) in white tissue culture treated 384 well plates. Test compounds are dissolved at 10 mM in DMSO then diluted into complete growth medium to 5× the final intended test concentration. These drug stock solutions, 5 μL/well are added to the tissue culture plate. The final DMSO concentration is 0.2%. The plates are briefly centrifuged then incubated overnight at 37° C. and 5% CO₂. To conduct the assay, the tissue culture plates are allowed to equilibrate to room temperature and One-Glo luciferase reagent (Promega, 25 μL/well) was added. The plates are briefly centrifuged then incubated at room temperature for 10 minutes. The luciferase intensity may be read on an Envision plate reader (Perkin Elmer). RORγ activity is determined relative to controls and plotted as a function of test compound concentration using PRISM (GraphPad) to determine a 50% effective concentration (EC₅₀). The luciferase signal in the absence of ursolic acid or test compound is defined at 100. The Max Response is the upper plateau in the signal as determined by line-fit using a 4-parameter logistic model in PRISM (GraphPad).

### Increasing the Amount of Interleukin-17 (IL-17) in a Subject

Further, it is contemplated that aryl indolinyl sulfonamide, aryl indolyl sulfone, and related compounds described herein, such as a compound of Formula I, I-A, I-A′, I-B, I-B′, I-C, I-D, II, II-A, II-B, or other compounds in Section I, can increase the amount of interleukin-17 (IL-17) in a subject. IL-17 is a cytokine that affects numerous biological functions. Accordingly, another aspect of the invention provides a method of increasing the amount of IL-17 in a subject. The method comprises administering to a subject an effective amount of an aryl indolinyl sulfonamide, aryl indolyl sulfone, or related compound described herein, such as a compound of Formula I, I-A, I-A′, I-B, I-B′, I-C, I-D, II, II-A, II-B, or other compounds in Section I, to increase the amount of IL-17 in the subject. In certain embodiments, the particular compound of Formula I, I-A, I-A′, I-B, I-B′, I-C, I-D, II, II-A, or II-B is the compound defined by one of the embodiments described above.

In certain embodiments, the subject is a human. In certain embodiments, administering the compound increases the amount of IL-17 produced by Th-17 cells in the subject. A change in the amount of IL-17 produced by, for example, Th-17 cells can be measured using procedures described in the literature, such as an ELISA assay or intracellular staining assay.

Further, it is contemplated that aryl indolinyl sulfonamide, aryl indolyl sulfone, and related compounds described herein, such as a compound of Formula I, I-A, I-A′, I-B, I-B′, I-C, I-D, II, II-A, II-B, or other compounds in Section I, may increase the synthesis of IL-17 in a subject. Accordingly, another aspect of the invention provides a method of increasing the synthesis of IL-17 in a subject. The method comprises administering to a subject an effective amount of a compound described herein, e.g., a compound of Formula I, I-A, I-A′, I-B, I-B′, I-C, I-D, II, II-A, II-B, or other compounds in Section I, to increase the synthesis of IL-17 in the subject. In certain embodiments, the particular compound of Formula I, I-A, I-A′, I-B, I-B′, I-C, I-D, II, II-A, or II-B is a compound defined by one of the embodiments described above.

### Adoptive Cellular Therapy

RORγ agonist compounds described herein may also be used in adoptive cellular therapy to treat various medical disorders, such as cancer, bacterial infections, fungal infections, and immune disorders. Cells, e.g., lymphocyte cells or dendritic cells, are exposed ex vivo to an RORγ agonist compound herein, and then the treated cells are administered to a patient. In adoptive cellular transfer, cells are obtained from a source (typically the patient in need of treatment), cultured ex vivo with an agent, and then the resulting cells are administered to the patient in need of therapy. The culturing typically subjects the cells to conditions whereby the cells increase in number (i.e., expansion) and/or acquire features providing improved therapeutic benefit. General features of the adoptive cellular therapy methods and compositions are described below, along with more specific embodiments of the lymphocyte cells, dendritic cells, and procedures for isolating and culturing cells.

Accordingly, one aspect of the invention provides a method of delivering to a patient a RORγ agonist treated cell selected from the group consisting of a lymphocyte cell and dendritic cell. The method comprises administering to a patient in need thereof a pharmaceutical composition comprising said cell that has been exposed ex vivo to an agonist of RORγ described herein, such as a compound of Formula I, I-A, I-A′, I-B, I-B′, I-C, I-D, II, II-A, or II-B. The method may further comprise a culturing step. In such embodiments, the method further comprises culturing a cell (i.e., the lymphocyte cell or dendritic cell) with an agonist of RORγ to provide the cell that has been exposed ex vivo to the agonist of RORγ. The culturing may comprise exposing the cell to a cytokine (e.g., IL-1(3, IL-2, IL-6, IL-7, IL-10, IL-12, IL-15, IL-18, IL-21, IL-23, or transforming growth factor beta). During the culturing step, the cell may be exposed to an antigen associated with a medical disorder. Although not to be bound by theory, cells having an receptor specific to an antigen associated with a medical disorder can provide a more effective therapy than cells lacking such a receptor. Accordingly, in certain embodiments, the culturing step comprises exposing the cell to an antigen associated with a medical disorder. The antigen may be an antigen presenting cell. Alternatively, the antigen may comprise cancer tissue. Further, as described below, the cell may be genetically altered to express a receptor specific to an antigen associated with a medical disorder.

The cell may be autologous or allogenic. Autologous cells are cells obtained from the patient who will receive the cell exposed ex vivo to an agonist of RORγ. As such, in certain embodiments, the method may further comprise obtaining a cell from said patient, for use in the culturing step. Alternatively, the cells may be allogenic, i.e., obtained from a subject that produces cells allogenic to cells of the patient. In such embodiments, the method may further comprise obtaining a cell from a subject that produces cells allogenic to lymphocyte cells of the patient, for use in the culturing step.

In certain embodiments, the cell is a lymphocyte cell. Lymphocyte cells can be obtained from human or animal tissues according to procedures described in the literature. In certain embodiments, the lymphocyte cell is obtained from blood, cancer tissue, bone marrow, the spleen, a lymph node, or the thymus. In certain other embodiments, the lymphocyte cell is obtained from a population of peripheral blood mononuclear cells, such as human peripheral blood mononuclear cells. In certain other embodiments, the lymphocyte cell is obtained from a lymph node in proximity to a tumor or site of infection. In certain other embodiments, the lymphocyte cell is obtained from cancer tissue. In yet other embodiments, the lymphocyte cell is a tumor-infiltrating-lymphocyte cell.

Cells can be characterized according to the presence of a receptor for an antigen specific for a medical disorder. In certain embodiments, the cell expresses a receptor for an antigen specific for a medical disorder. As indicate above, such cells may provide more effective therapies for treating disease since the cells are more likely to target tissue specific to the disease to be treated. In certain embodiments, the medical disorder is a cancer, bacterial infection, fungal infection, or immune disorder. In yet other embodiments, the cell may express a receptor that, while not specific for a medical disorder, has utility in enhancing cell efficacy in treating the disorder.

Various types of lymphocyte cells have been described in the literature for use in adoptive cellular transfer. In certain embodiments, the lymphocyte cell is a T cell. In certain other embodiments, the lymphocyte cell is a CD8⁺ T cell, CD4⁺ T cell, or T_{H}17 cell. In certain other embodiments, the lymphocyte cell is a CD8⁺ T cell, CD4⁺ T cell, or a combination thereof. In certain other embodiments, the lymphocyte cell is a natural killer cell. In certain other embodiments, the lymphocyte cell is a Tc17 cell, natural killer T cell, or γδ T cell. In yet other embodiments, the lymphocyte cell is a genetically altered lymphocyte cell.

Cells may be administered to the patient according to procedures described in the literature. In certain embodiments, the administering comprises injecting into the patient the cells. The injecting may be intravenous injection or injection directly into diseased tissue, such as a tumor. In yet other embodiments, the injecting may be subcutaneous injection into the patient.

The therapeutic method embraces combination therapies, such as administering (i) an agent that enhances the efficacy of the cell exposed to the agonist of RORγ and/or (ii) an agent having independent efficacy in treating the target medical disorder.

Another aspect of the invention provides a method of preparing a population of cells that have been exposed ex vivo to an agonist of RORγ described herein, where the cells are lymphocyte cells and/or dendritic cells. The method comprises exposing a population of cells selected from the group consisting of lymphocyte cells and dendritic cells ex vivo to an agonist of RORγ described herein to thereby provide said population of cells that have been exposed ex vivo to an agonist of RORγ. The population of cells may be used in therapeutic methods described herein. The exposing step may comprise culturing a population of cells with the agonist of RORγ for a duration of time sufficient to increase the number of cells in the population. The culturing may comprise exposing the cell to a cytokine (e.g., IL-13, IL-2, IL-6, IL-7, IL-10, IL-12, IL-15, IL-18, IL-21, IL-23, or transforming growth factor beta). Further during the culturing step, the cell may optionally be exposed to an antigen associated with a medical disorder. Accordingly, in certain embodiments, the culturing step comprises exposing the cell to an antigen associated with a medical disorder. The antigen may be an antigen presenting cell. Alternatively, the antigen may comprise cancer tissue. The cell may be autologous or allogenic. Autologous cells are cells obtained from the patient whom will receive the cell exposed ex vivo to an agonist of RORγ. As such, in certain embodiments, the method may further comprise obtaining a cell (i.e., a lymphocyte or dendritic cell) from said patient for use in the culturing step. Alternatively, the cells may be allogenic, i.e., obtained from subject that produces cells allogenic to cells of the patient. In such embodiments, the method may further comprise obtaining a cell from a subject that produces cells allogenic to cells of the patient, for use in the culturing step. In certain embodiments, the cell is a lymphocyte cell. Lymphocyte cells can be obtained from human or animal tissues according to procedures described in the literature. In certain embodiments, the lymphocyte cell is obtained from blood, cancer tissue, bone marrow, the spleen, a lymph node, or the thymus. In certain other embodiments, the lymphocyte cell is obtained from a population of peripheral blood mononuclear cells, such as human peripheral blood mononuclear cells. In certain other embodiments, the lymphocyte cell is obtained from a lymph node in proximity to a tumor or site of infection. In certain other embodiments, the lymphocyte cell is obtained from cancer tissue. In yet other embodiments, the lymphocyte cell is a tumor-infiltrating-lymphocyte cell.

Cells can be characterized according to the presence of a receptor for an antigen specific for a medical disorder. In certain embodiments, the cell expresses a receptor for an antigen specific for a medical disorder. As indicated above, such cells may provide more effective therapies for treating disease since the cells is more likely to target tissue specific to the disease to be treated. In certain embodiments, the medical disorder is a cancer, bacterial infection, fungal infection, or immune disorder. In yet other embodiments, the cell may express a receptor that, while not specific for a medical disorder, has utility in enhancing cell efficacy in treating the disorder.

As described above, various types of lymphocyte cells have been described in the literature for use in adoptive cellular transfer. In certain embodiments, the lymphocyte cell is a T cell. In certain other embodiments, the lymphocyte cell is a CD8⁺ T cell, CD4⁺ T cell, or T_{H}17 cell. In certain other embodiments, the lymphocyte cell is a CD8⁺ T cell, CD4⁺ T cell, or a combination thereof. In certain other embodiments, the lymphocyte cell is a natural killer cell. In certain other embodiments, the lymphocyte cell is a Tc17, natural killer T cell, or γ6 T cell. In yet other embodiments, the lymphocyte cell is a genetically altered lymphocyte cell.

Another aspect of the invention provides a method of treating a medical disorder. The method comprises administering to a patient in need thereof a cell that has been exposed ex vivo to an agonist of RORγ described herein to treat the medical disorder, wherein the cell is a lymphocyte cell or dendritic cell. The medical disorder can be, for example, a cancer, bacterial infection, fungal infection, or immune disorder. Additional exemplary medical disorders are described above, and in certain embodiments, the medical disorder is a cancer selected from the group consisting of a solid tumor, lymphoma, and leukemia. In certain other embodiments, the medical disorder is a cancer selected from the group consisting of ovarian cancer, melanoma, colorectal cancer, lung cancer, breast cancer, prostate cancer, pancreatic cancer, renal cell carcinoma, leukemia, a B-cell lymphoma, and non-Hodgkin lymphoma.

Another aspect of the invention provides a population of lymphocyte cells that have been exposed ex vivo to an agonist of RORγ described herein. The population may be characterized by the presence and/or quantity of particular types of cells in the population. For example, in certain embodiments, the population comprises one or more of the following: T cells and natural killer cells. In certain other embodiments, a majority of lymphocyte cells in the population are T cells. In certain other embodiments, a majority of lymphocyte cells in the population are CD8⁺ T cells, CD4⁺ T cells, T_{H}17 cells, or a combination thereof. In yet other embodiments, a majority of lymphocyte cells in the population are natural killer cells. In yet other embodiments, a single type of lymphocyte cell (e.g., a T cell, CD8⁺ T cell, CD4⁺ T cell, T_{H}17 cell, Tc17 cell, natural killer T cell, or γ6 T cell) comprises at least 60%, 70% 80%, 90% or 95% of the cells in the population. In yet other embodiments, the population is characterized by: (i) a majority of lymphocyte cells in the population are T cells, (ii) a majority of lymphocyte cells in the population are CD8⁺ T cells, CD4⁺ T cells, T_{H}17 cells, or a combination thereof, (iii) a majority of lymphocyte cells in the population are Tc17 cells, (iv) a majority of lymphocyte cells in the population are natural killer cells, or (v) a majority of lymphocyte cells in the population are natural killer T cells, γ6 T cells, or a combination thereof. In yet other embodiments, a majority of lymphocyte cells in the population are CD8⁺ T cells, CD4⁺ T cells, or a combination thereof. In yet other embodiments, the population is characterized by a majority of lymphocyte cells in the population are Tc17 cells, CD4+ Th0 T lymphocyte cells, Th17-polarized CD4+ T lymphocyte cells, CD8+ Tc17 T lymphocyte cells, or a combination thereof.

In each of the above aspects and embodiments, lymphocyte cells may be characterized according to whether they are a tumor infiltrating lymphocyte, naïve T lymphocyte, memory T lymphocyte, effector T lymphocyte, CD8⁺ T cell, CD4⁺ T cell, CD4⁺/CD8⁺ double positive T lymphocyte, CD28⁺CD8⁺ T cell, or T_{H}17 cell. CD8⁺ T cells can be separated into naïve CD8⁺ T cells, memory CD8⁺ T cells, and effector CD8⁺ T cells, according to cell surface antigens characteristic to each type of cell. Whether a cell or cell population is positive for a particular cell surface marker can be determined by flow cytometry using staining with a specific antibody for the surface marker and an isotype matched control antibody. A cell population negative for a marker refers to the absence of significant staining of the cell population with the specific antibody above the isotype control, and positive refers to uniform staining of the cell population above the isotype control. For instance, CD4⁺ T helper cells can be sorted into naive, central memory, and effector cells by identifying cell populations that have cell surface antigens. In certain embodiments, central memory CD4+ T cells are CD62L positive and CD45RO positive. In certain embodiments, effector CD4⁺ T cells are CD62L and CD45RO negative. In yet other embodiments, the lymphocyte cell is a Th1 cell, Tc1 cell, Th0 cell, or Tc0 cell. In certain embodiments, the lymphocyte cell is a CD8⁺ T cell, which is optionally further characterized according to the whether the CD8⁺ T cell is a naïve CD8⁺ T cell, a memory CD8⁺ T cell, or an effector CD8⁺ T cell. In certain embodiments, the lymphocyte cell is a memory CD8⁺ T cell, which may be further characterized according to whether the cell is CD62L positive or CD45RO positive. In certain other embodiments, the lymphocyte cell is an effector CD8⁺ T cell, which may be further characterized according to whether the cell is CD62L negative or CD45RO negative. In yet other embodiments, the lymphocyte cell is a CD4+ Th0 T lymphocyte, Th17-polarized CD4+ T lymphocyte, or CD8+ Tc17 T lymphocyte. In still other embodiments, the lymphocyte cell is a memory T cell present in CD62L+ or CD62L− subsets of CD8+ peripheral blood lymphocytes. In certain embodiments, the central memory T cells may be CD45RO+, CD62L+, CD8+ T cells. In certain embodiments, effector T cells are negative for CD62L, CCR7, CD28, and CD127, and positive for granzyme B and perforin.

T cells can be characterized according to the identity of a T cell receptor located on the surface of the T cell. The T cell receptor is a disulfide-linked membrane-anchored heterodimer that normally consists of highly variable alpha (a) and beta (j) chains expressed as part of a complex with the invariant CD3 chain molecules. T cells expressing this receptor are referred to as α:β (or αβ) T cells. A minority of T cells express an alternate receptor, formed by variable gamma (γ) and delta (δ) chains, and such T cells are referred as γδ T cells. One subtype of T cells is natural killer T (NKT) cells. NKT cells are a heterogeneous group of T cells that share properties of both T cells and natural killer NK cells. Many NKT cells recognize the non-polymorphic CD1d molecule, an antigen-presenting molecule that binds self- and foreign lipids and glycolipids. Other subtypes of T cells include, for example, CD8⁺ T cells, CD4⁺ T cells, Tc17 cells, natural killer T cells, and γδ T cells. Still other subtypes of T cells include, for example, CD4⁻CD8⁻ T cells and CD28⁺CD8⁺ T cells.

Preferably the lymphocyte cell comprises a receptor specific for an antigen of a medical condition. The receptor can be the endogenous lymphocyte cell receptor, i.e., the antigen-specific lymphocyte cell receptor that is endogenous (i.e., native to) the lymphocyte. In such instances, the lymphocyte comprising the endogenous lymphocyte cell receptor can be a lymphocyte cell that was isolated from the patient, which is known to express the particular medical condition-specific antigen. Alternatively, the lymphocyte comprising the endogenous lymphocyte cell receptor can be a lymphocyte cell that was isolated from a subject that produces allogenic lymphocyte cells (i.e., lymphocyte cells that are histocompatible with the patient that will receive the lymphocyte cells). In certain embodiments, the subject from which lymphocyte cells are obtained may be immunized prior to obtaining the lymphocyte cells, so that the lymphocyte cells to be administered to the patient will have specificity for the medical disorder to be treated.

The antigen of a disease recognized by the endogenous lymphocyte cell receptor can be any antigen which is characteristic of the disease. For example, the antigen may be, for example, a tumor antigen, such as gp100, MART-1, TRP-1, TRP-2, tyrosinase, NY-ESO-1, MAGE-1, or MAGE-3.

Lymphocyte cells may also be characterized according to the presence of a phenotypic marker of activation for tumor reactivity, such as the presence of 4-1BBL. Populations of lymphocyte cells enriched for such a phenotypic marker may provide therapeutic advantages. Lymphocyte cells may also be characterized according to the level of expression of the RORγ. In certain embodiments, the lymphocyte cell may be induced to express or engineered to express RORγ, thereby increasing the amount of RORγ.

The lymphocyte cell may be a genetically modified lymphocyte cell, such as a genetically modified lymphocyte cell described in, for example, International Patent Application Publication No. WO 2012/129514, which is hereby incorporated by reference. Genetic modification of the lymphocyte may improve the efficacy of therapy by promoting the viability and/or function of transferred lymphocyte cells, provide a genetic marker to permit selection and/or evaluation of in vivo survival or migration, or may incorporate functions that improve the safety of immunotherapy, for example, by making the cell susceptible to negative selection in vivo. The lymphocyte may be genetically modified so that the lymphocyte cell expresses certain proteins, such as a survival cytokine (e.g., granulocyte-macrophage colony-stimulating factor) and/or receptor for an antigen (e.g., a tumor antigen).

Accordingly, in embodiments, lymphocyte cells are modified with chimeric antigen receptors (CAR). The CARs may comprise a single-chain antibody fragment (scFv) that is derived from the variable heavy (VH) and variable light (VL) chains of a monoclonal antibody (mAb) linked to the TCR CD3⁺ chain that mediates T-cell activation and cytotoxicity. Costimulatory signals can also be provided through the CAR by fusing the costimulatory domain of CD28 or 4-1 BB to the CD3⁺ chain. CARs are specific for cell surface molecules independent from HLA, thus overcoming the limitations of TCR-recognition including HLA-restriction and low levels of HLA-expression on tumor cells.

### III. Combination Therapy

Another aspect of the invention provides for combination therapy. Aryl indolinyl sulfonamide, aryl indolyl sulfone, and related compounds (e.g., a compound of Formula I, I-A, I-A′, I-B, I-B′, I-C, I-D, II, II-A, II-B, or other compounds in Section I) or their pharmaceutically acceptable salts may be used in combination with additional therapeutic agents to treat medical disorders, such as a cancer, bacterial infection, fungal infection, and immune deficiency disorder.

Exemplary therapeutic agents that may be used as part of a combination therapy in treating cancer, include, for example, mitomycin, tretinoin, ribomustin, gemcitabine, vincristine, etoposide, cladribine, mitobronitol, methotrexate, doxorubicin, carboquone, pentostatin, nitracrine, zinostatin, cetrorelix, letrozole, raltitrexed, daunorubicin, fadrozole, fotemustine, thymalfasin, sobuzoxane, nedaplatin, cytarabine, bicalutamide, vinorelbine, vesnarinone, aminoglutethimide, amsacrine, proglumide, elliptinium acetate, ketanserin, doxifluridine, etretinate, isotretinoin, streptozocin, nimustine, vindesine, flutamide, drogenil, butocin, carmofur, razoxane, sizofilan, carboplatin, mitolactol, tegafur, ifosfamide, prednimustine, picibanil, levamisole, teniposide, improsulfan, enocitabine, lisuride, oxymetholone, tamoxifen, progesterone, mepitiostane, epitiostanol, formestane, interferon-alpha, interferon-2 alpha, interferon-beta, interferon-gamma, colony stimulating factor-1, colony stimulating factor-2, denileukin diftitox, interleukin-2, and leutinizing hormone releasing factor.

An additional class of agents that may be used as part of a combination therapy in treating cancer is immune checkpoint inhibitors (also referred to as immune checkpoint blockers). Immune checkpoint inhibitors are a class of therapeutic agents that have the effect of blocking immune checkpoints. See, for example, Pardoll in Nature Reviews Cancer (2012) vol. 12, pages 252-264. Exemplary immune checkpoint inhibitors include agents that inhibit one or more of (i) cytotoxic T-lymphocyte-associated antigen 4 (CTLA4), (ii) programmed cell death protein 1 (PD1), (iii) PDL1, (iv) LAB3, (v) B7-H3, (vi) B7-H4, and (vii) TIM3. The CTLA4 inhibitor Ipilumumab has been approved by the United States Food and Drug Administration for treating melanoma.

Yet other agents that may be used as part of a combination therapy in treating cancer are monoclonal antibody agents that target non-checkpoint targets (e.g., herceptin) and non-cytoxic agents (e.g., tyrosine-kinase inhibitors).

Accordingly, another aspect of the invention provides a method of treating cancer in a patient, where the method comprises administering to the patient in need thereof (i) a therapeutically effective amount of a RORγ agonist compound described herein and (ii) a second anti-cancer agent, in order to treat the cancer, where the second therapeutic agent may be one of the additional therapeutic agents described above (e.g., mitomycin, tretinoin, ribomustin, gemcitabine, an immune checkpoint inhibitor, or a monoclonal antibody agent that targets non-checkpoint targets) or one of the following:
an inhibitor selected from an ALK Inhibitor, an ATR Inhibitor, an A2A Antagonist, a Base Excision Repair Inhibitor, a Bcr-Abl Tyrosine Kinase Inhibitor, a Bruton's Tyrosine Kinase Inhibitor, a CDC7 Inhibitor, a CHK1 Inhibitor, a Cyclin-Dependent Kinase Inhibitor, a DNA-PK Inhibitor, an Inhibitor of both DNA-PK and mTOR, a DNMT1 Inhibitor, a DNMT1 Inhibitor plus 2-chloro-deoxyadenosine, an HDAC Inhibitor, a Hedgehog Signaling Pathway Inhibitor, an IDO Inhibitor, a JAK Inhibitor, a mTOR Inhibitor, a MEK Inhibitor, a MELK Inhibitor, a MTH1 Inhibitor, a PARP Inhibitor, a Phosphoinositide 3-Kinase Inhibitor, an Inhibitor of both PARP1 and DHODH, a Proteasome Inhibitor, a Topoisomerase-II Inhibitor, a Tyrosine Kinase Inhibitor, a VEGFR Inhibitor, and a WEE1 Inhibitor;
an agonist of OX40, CD137, CD40, GITR, CD27, HVEM, TNFRSF25, or ICOS;
a therapeutic antibody targeting one of the following: CD20, CD30, CD33, CD52, EpCAM, CEA, gpA33, a mucin, TAG-72, CAIX, PSMA, a folate-binding protein, a ganglioside, Le, VEGF, VEGFR, VEGFR2, integrin aV33, integrin a5131, EGFR, ERBB2, ERBB3, MET, IGF1R, EPHA3, TRAILR1, TRAILR2, RANKL, FAP, tenascin, CD19, KIR, NKG2A, CD47, CEACAM1, c-MET, VISTA, CD73, CD38, BAFF, interleukin-1 beta, B4GALNT1, interleukin-6, and interleukin-6 receptor;
a cytokine selected from IL-12, IL-15, GM-CSF, and G-CSF;
a therapeutic agent selected from sipuleucel-T, aldesleukin (a human recombinant interleukin-2 product having the chemical name des-alanyl-1, serine-125 human interleukin-2), dabrafenib (a kinase inhibitor having the chemical name N-{3-[5-(2-aminopyrimidin-4-yl)-2-tert-butyl-1,3-thiazol-4-yl]-2-fluorophenyl}-2,6-difluorobenzenesulfonamide), vemurafenib (a kinase inhibitor having the chemical name propane-1-sulfonic acid {3-[5-(4-chlorophenyl)-1H-pyrrolo[2,3-b]pyridine-3-carbonyl]-2,4-difluoro-phenyl}-amide), and 2-chloro-deoxyadenosine; or
a placental growth factor, an antibody-drug conjugate, an oncolytic virus, or an anti-cancer vaccine.

In certain embodiments, the second anti-cancer agent is an ALK Inhibitor. In certain embodiments, the second anti-cancer agent is an ALK Inhibitor comprising ceritinib or crizotinib. In certain embodiments, the second anti-cancer agent is an ATR Inhibitor. In certain embodiments, the second anti-cancer agent is an ATR Inhibitor comprising AZD6738 or VX-970. In certain embodiments, the second anti-cancer agent is an A2A Antagonist. In certain embodiments, the second anti-cancer agent is a Base Excision Repair Inhibitor comprising methoxyamine. In certain embodiments, the second anti-cancer agent is a Base Excision Repair Inhibitor, such as methoxyamine. In certain embodiments, the second anti-cancer agent is a Bcr-Abl Tyrosine Kinase Inhibitor. In certain embodiments, the second anti-cancer agent is a Bcr-Abl Tyrosine Kinase Inhibitor comprising dasatinib or nilotinib. In certain embodiments, the second anti-cancer agent is a Bruton's Tyrosine Kinase Inhibitor. In certain embodiments, the second anti-cancer agent is a Bruton's Tyrosine Kinase Inhibitor comprising ibrutinib. In certain embodiments, the second anti-cancer agent is a CDC7 Inhibitor. In certain embodiments, the second anti-cancer agent is a CDC7 Inhibitor comprising RXDX-103 or AS-141.

In certain embodiments, the second anti-cancer agent is a CHK1 Inhibitor. In certain embodiments, the second anti-cancer agent is a CHK1 Inhibitor comprising MK-8776, ARRY-575, or SAR-020106. In certain embodiments, the second anti-cancer agent is a Cyclin-Dependent Kinase Inhibitor. In certain embodiments, the second anti-cancer agent is a Cyclin-Dependent Kinase Inhibitor comprising palbociclib. In certain embodiments, the second anti-cancer agent is a DNA-PK Inhibitor. In certain embodiments, the second anti-cancer agent is a DNA-PK Inhibitor comprising MSC2490484A. In certain embodiments, the second anti-cancer agent is Inhibitor of both DNA-PK and mTOR. In certain embodiments, the second anti-cancer agent comprises CC-115.

In certain embodiments, the second anti-cancer agent is a DNMT1 Inhibitor. In certain embodiments, the second anti-cancer agent is a DNMT1 Inhibitor comprising decitabine, RX-3117, guadecitabine, NUC-8000, or azacytidine. In certain embodiments, the second anti-cancer agent comprises a DNMT1 Inhibitor and 2-chloro-deoxyadenosine. In certain embodiments, the second anti-cancer agent comprises ASTX-727.

In certain embodiments, the second anti-cancer agent is a HDAC Inhibitor. In certain embodiments, the second anti-cancer agent is a HDAC Inhibitor comprising OBP-801, CHR-3996, etinostate, resminostate, pracinostat, CG-200745, panobinostat, romidepsin, mocetinostat, belinostat, AR-42, ricolinostat, KA-3000, or ACY-241.

In certain embodiments, the second anti-cancer agent is a Hedgehog Signaling Pathway Inhibitor. In certain embodiments, the second anti-cancer agent is a Hedgehog Signaling Pathway Inhibitor comprising sonidegib or vismodegib. In certain embodiments, the second anti-cancer agent is an IDO Inhibitor. In certain embodiments, the second anti-cancer agent is an IDO Inhibitor comprising INCB024360. In certain embodiments, the second anti-cancer agent is a JAK Inhibitor. In certain embodiments, the second anti-cancer agent is a JAK Inhibitor comprising ruxolitinib or tofacitinib. In certain embodiments, the second anti-cancer agent is a mTOR Inhibitor. In certain embodiments, the second anti-cancer agent is a mTOR Inhibitor comprising everolimus or temsirolimus. In certain embodiments, the second anti-cancer agent is a MEK Inhibitor. In certain embodiments, the second anti-cancer agent is a MEK Inhibitor comprising cobimetinib or trametinib. In certain embodiments, the second anti-cancer agent is a MELK Inhibitor. In certain embodiments, the second anti-cancer agent is a MELK Inhibitor comprising ARN-7016, APTO-500, or OTS-167. In certain embodiments, the second anti-cancer agent is a MTH1 Inhibitor. In certain embodiments, the second anti-cancer agent is a MTH1 Inhibitor comprising (S)-crizotinib, TH287, or TH588.

In certain embodiments, the second anti-cancer agent is a PARP Inhibitor. In certain embodiments, the second anti-cancer agent is a PARP Inhibitor comprising MP-124, olaparib, BGB-290, talazoparib, veliparib, niraparib, E7449, rucaparb, or ABT-767. In certain embodiments, the second anti-cancer agent is a Phosphoinositide 3-Kinase Inhibitor. In certain embodiments, the second anti-cancer agent is a Phosphoinositide 3-Kinase Inhibitor comprising idelalisib. In certain embodiments, the second anti-cancer agent is an inhibitor of both PARP1 and DHODH (i.e., an agent that inhibits both poly ADP ribose polymerase 1 and dihydroorotate dehydrogenase).

In certain embodiments, the second anti-cancer agent is a Proteasome Inhibitor. In certain embodiments, the second anti-cancer agent is a Proteasome Inhibitor comprising bortezomib or carfilzomib. In certain embodiments, the second anti-cancer agent is a Topoisomerase-II Inhibitor. In certain embodiments, the second anti-cancer agent is a Topoisomerase-II Inhibitor comprising vosaroxin.

In certain embodiments, the second anti-cancer agent is a Tyrosine Kinase Inhibitor. In certain embodiments, the second anti-cancer agent is a Tyrosine Kinase Inhibitor comprising bosutinib, cabozantinib, imatinib or ponatinib. In certain embodiments, the second anti-cancer agent is a VEGFR Inhibitor. In certain embodiments, the second anti-cancer agent is a VEGFR Inhibitor comprising regorafenib. In certain embodiments, the second anti-cancer agent is a WEE1 Inhibitor. In certain embodiments, the second anti-cancer agent is a WEE1 Inhibitor comprising AZD1775.

In certain embodiments, the second anti-cancer agent is an agonist of OX40, CD137, CD40, GITR, CD27, HVEM, TNFRSF25, or ICOS. In certain embodiments, the second anti-cancer agent is a therapeutic antibody selected from the group consisting of rituximab, ibritumomab tiuxetan, tositumomab, obinutuzumab, ofatumumab, brentuximab vedotin, gemtuzumab ozogamicin, alemtuzumab, IGN101, adecatumumab, labetuzumab, huA33, pemtumomab, oregovomab, minetumomab, cG250, J591, Mov18, farletuzumab, 3F8, ch14.18, KW-2871, hu3S193, lgN311, bevacizumab, IM-2C6, pazopanib, sorafenib, axitinib, CDP791, lenvatinib, ramucirumab, etaracizumab, volociximab, cetuximab, panitumumab, nimotuzumab, 806, afatinib, erlotinib, gefitinib, osimertinib, vandetanib, trastuzumab, pertuzumab, MM-121, AMG 102, METMAB, SCH 900105, AVE1642, IMC-A12, MK-0646, R1507, CP 751871, KB004, IIIA-4, mapatumumab, HGS-ETR2, CS-1008, denosumab, sibrotuzumab, F19, 81C6, MEDI551, lirilumab, MEDI9447, daratumumab, belimumab, canakinumab, dinutuximab, siltuximab, and tocilizumab.

In certain embodiments, the second anti-cancer agent is a placental growth factor. In certain embodiments, the second anti-cancer agent is a placental growth factor comprising ziv-aflibercept. In certain embodiments, the second anti-cancer agent is an antibody-drug conjugate. In certain embodiments, the second anti-cancer agent is an antibody-drug conjugate selected from the group consisting of brentoxumab vedotin and trastuzumab emtransine.

In certain embodiments, the second anti-cancer agent is an oncolytic virus. In certain embodiments, the second anti-cancer agent is the oncolytic virus talimogene laherparepvec. In certain embodiments, the second anti-cancer agent is an anti-cancer vaccine. In certain embodiments, the second anti-cancer agent is an anti-cancer vaccine selected from the group consistint of a GM-CSF tumor vaccine, a STING/GM-CSF tumor vaccine, and NY-ESO-1. In certain embodiments, the second anti-cancer agent is a cytokine selected from IL-12, IL-15, GM-CSF, and G-CSF.

In certain embodiments, the second anti-cancer agent is a therapeutic agent selected from sipuleucel-T, aldesleukin (a human recombinant interleukin-2 product having the chemical name des-alanyl-1, serine-125 human interleukin-2), dabrafenib (a kinase inhibitor having the chemical name N-{3-[5-(2-aminopyrimidin-4-yl)-2-tert-butyl-1,3-thiazol-4-yl]-2-fluorophenyl}-2,6-difluorobenzenesulfonamide), vemurafenib (a kinase inhibitor having the chemical name propane-1-sulfonic acid {3-[5-(4-chlorophenyl)-1H-pyrrolo[2,3-b]pyridine-3-carbonyl]-2,4-difluoro-phenyl}-amide), and 2-chloro-deoxyadenosine.

Exemplary therapeutic agents that may be used as part of a combination therapy in treating a bacterial infection, include, for example, amoxicillin, azithromycin, cefazolin, ceftriaxone, cefuroxime, cephalexin, ciprofloxacin, clindamycin, doxycycline, levofloxacin, linezolid, metronidazole, moxifloxacin, and penicillin.

Exemplary therapeutic agents that may be used as part of a combination therapy in treating a fungal infection, include, for example, 2-phenylphenol; 8-hydroxyquinoline sulfate; acibenzolar-S-methyl; aldimorph; amidoflumet; ampropylfos; ampropylfos-potassium; andoprim; anilazine; azaconazole; azoxystrobin; benalaxyl; benodanil; benomyl; benthiavalicarb-isopropyl; benzamacril; benzamacril-isobutyl; bilanafos; binapacryl; biphenyl; bitertanol; blasticidin-S; bromuconazole; butylamine; calcium polysulphide; capsimycin; captafol; captan; carbendazim; carboxin; carpropamid; carvone; chinomethionat; chlobenthiazone; chlorfenazole; chloroneb; chlorothalonil; chlozolinate; clozylacon; cyazofamid; cyflufenamid; cymoxanil; cyproconazole; cyprodinil; cyprofuram; Dagger G; debacarb; dichlofluanid; dichlone; dichlorophen; diclocymet; diclomezine; dicloran; diethofencarb; difenoconazole; diflumetorim; dimethirimol; dimethomorph; dimoxystrobin; diniconazole; diniconazole-M; dinocap; diphenylamine; dipyrithione; ditalimfos; dithianon; dodine; drazoxolon; edifenphos; epoxiconazole; ethaboxam; ethirimol; etridiazole; famoxadone; fenamidone; fenapanil; fenarimol; fenbuconazole; fenfuram; fenhexamid; fenitropan; fenoxanil; fenpiclonil; fenpropidin; fenpropimorph; ferbam; fluazinam; flubenzimine; fludioxonil; flumetover; flumorph; fluoromide; fluoxastrobin; fluquinconazole; flurprimidol; flusilazole; flusulphamide, hexaconazole; hymexazole; imazalil; imibenconazole; iminoctadine triacetate; iminoctadine tris(albesil); iodocarb; ipconazole; iprobenfos; iprodione; iprovalicarb; irumamycin; isoprothiolane; isovaledione; kasugamycin; kresoxim-methyl; oxyfenthiin; paclobutrazole; pefurazoate; penconazole; pencycuron; phosdiphen; phthalide; picoxystrobin; piperalin; polyoxins; polyoxorim; probenazole; prochloraz; procymidone; propamocarb; propanosine-sodium; propiconazole; propineb; proquinazid; prothioconazole; pyraclostrobin; pyrazophos; pyrifenox; pyrimethanil; pyroquilon; pyroxyfur; pyrrolenitrine; tetraconazole; thiabendazole; thicyofen; thifluzamide; thiophanate-methyl; thiram; tioxymid; tricyclazole; tridemorph; trifloxystrobin; triflumizole; triforine; triticonazole; uniconazole; validamycin A; vinclozolin; zineb; ziram; and zoxamide.

The amount of aryl indolinyl sulfonamide, aryl indolyl sulfone, or related compound (e.g., a compound of Formula I, I-A, I-A′, I-B, I-B′, I-C, I-D, II, II-A, II-B, or other compounds in Section I) and additional therapeutic agent and the relative timing of administration may be selected in order to achieve a desired combined therapeutic effect. For example, when administering a combination therapy to a patient in need of such administration, the therapeutic agents in the combination, or a pharmaceutical composition or compositions comprising the therapeutic agents, may be administered in any order such as, for example, sequentially, concurrently, together, simultaneously and the like. Further, for example, an aryl indolinyl sulfonamide, aryl indolyl sulfone, or related compound (e.g., a compound of any one of Formula I, I-A, I-A′, I-B, I-B′, I-C, I-D, II, II-A, II-B, or other compounds in Section I) may be administered during a time when the additional therapeutic agent(s) exerts its prophylactic or therapeutic effect, or vice versa.

The doses and dosage regimen of the active ingredients used in the combination therapy may be determined by an attending clinician. In certain embodiments, the aryl indolinyl sulfonamide, aryl indolyl sulfone, or related compound (e.g., a compound of any one of Formula I, I-A, I-A′, I-B, I-B′, I-C, I-D, II, II-A, II-B, or other compounds in Section I) and the additional therapeutic agent(s) are administered in doses commonly employed when such agents are used as monotherapy for treating the disorder. In other embodiments, the aryl indolinyl sulfonamide, aryl indolyl sulfone, or related compound (e.g., a compound of any one of Formula I, I-A, I-A′, I-B, I-B′, I-C, I-D, II, II-A, II-B, or other compounds in Section I) and the additional therapeutic agent(s) are administered in doses lower than the doses commonly employed when such agents are used as monotherapy for treating the disorder. In certain embodiments, the aryl indolinyl sulfonamide, aryl indolyl sulfone, or related compound (e.g., a compound of any one of Formula I, I-A, I-A′, I-B, I-B′, I-C, I-D, II, II-A, II-B, or other compounds in Section I) and the additional therapeutic agent(s) are present in the same composition, which is suitable for oral administration.

In certain embodiments, the aryl indolinyl sulfonamide, aryl indolyl sulfone, or related compound (e.g., a compound of any one of Formula I, I-A, I-A′, I-B, I-B′, I-C, I-D, II, II-A, II-B, or other compounds in Section I) and the additional therapeutic agent(s) may act additively or synergistically. A synergistic combination may allow the use of lower dosages of one or more agents and/or less frequent administration of one or more agents of a combination therapy. A lower dosage or less frequent administration of one or more agents may lower toxicity of the therapy without reducing the efficacy of the therapy.

Another aspect of this invention is a kit comprising a therapeutically effective amount of the aryl indolinyl sulfonamide, aryl indolyl sulfone, or related compound (e.g., a compound of any one of Formula I, I-A, I-A′, I-B, I-B′, I-C, I-D, II, II-A, II-B, or other compounds in Section I), a pharmaceutically acceptable carrier, vehicle or diluent, and optionally at least one additional therapeutic agent listed above.

### IV. Pharmaceutical Compositions and Dosing Considerations

As indicated above, the invention provides pharmaceutical compositions, which comprise a therapeutically-effective amount of one or more of the compounds described above, formulated together with one or more pharmaceutically acceptable carriers (additives) and/or diluents. The pharmaceutical compositions may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally.

The phrase “therapeutically-effective amount” as used herein means that amount of a compound, material, or composition comprising a compound of the present invention which is effective for producing some desired therapeutic effect in at least a sub-population of cells in an animal at a reasonable benefit/risk ratio applicable to any medical treatment.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 0.1 percent to about ninety-nine percent of active ingredient, preferably from about 5 percent to about 70 percent, most preferably from about 10 percent to about 30 percent.

In certain embodiments, a formulation of the present invention comprises an excipient selected from the group consisting of cyclodextrins, celluloses, liposomes, micelle forming agents, e.g., bile acids, and polymeric carriers, e.g., polyesters and polyanhydrides; and a compound of the present invention. In certain embodiments, an aforementioned formulation renders orally bioavailable a compound of the present invention.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules, trouches and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the compounds of the present invention are administered as pharmaceuticals, to humans and animals, they can be given per se or as a pharmaceutical composition containing, for example, 0.1 to 99% (more preferably, 10 to 30%) of active ingredient in combination with a pharmaceutically acceptable carrier.

The preparations of the present invention may be given orally, parenterally, topically, or rectally. They are of course given in forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Oral administrations are preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracistemally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically-acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above. Preferably, the compounds are administered at about 0.01 mg/kg to about 200 mg/kg, more preferably at about 0.1 mg/kg to about 100 mg/kg, even more preferably at about 0.5 mg/kg to about 50 mg/kg. When the compounds described herein are co-administered with another agent (e.g., as sensitizing agents), the effective amount may be less than when the agent is used alone.

If desired, the effective daily dose of the active compound may be administered as two, three, four, five, six or more sub-doses administered separately at appropriate intervals throughout the day, optionally, in unit dosage forms. Preferred dosing is one administration per day.

The invention further provides a unit dosage form (such as a tablet or capsule) comprising an aryl indolinyl sulfonamide, aryl indolyl sulfone, or related compound described herein in a therapeutically effective amount for the treatment of a medical disorder described herein.

### EXAMPLES

The invention now being generally described, will be more readily understood by reference to the following examples, which are included merely for purposes of illustration of certain aspects and embodiments of the present invention, and are not intended to limit the invention. Starting materials described herein can be obtained from commercial sources or may be readily prepared from commercially available materials using transformations known to those of skill in the art.

### Example 1—Synthesis of 5-(2,5-difluorophenyl)-3-((3-(trifluoromethyl)phenyl)sulfonyl)-2,3-dihydrobenzo[d]oxazole

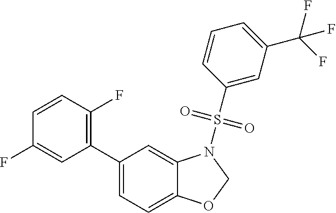 [SMILES: O=S(=O)(c1cccc(C(F)(F)F)c1)N1COc2ccc(-c3cc(F)ccc3F)cc21]

### Part I—Synthesis of 3-amino-2′,5′-difluoro-[1,1′-biphenyl]-4-ol

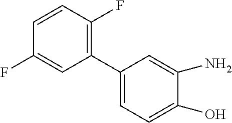 [SMILES: Nc1cc(-c2cc(F)ccc2F)ccc1O]

In a vial was combined (2,5-difluorophenyl)boronic acid (1.9 g, 12 mmol), [1,1′-bis(diphenylphosphino)ferrocene]dichloropalladium(II) complex with dichloromethane (0.61 g, 0.80 mmol), and 3M potassium carbonate (8 mL, 16 mmol) in 1,4-dioxane (50 mL). 2-Amino-4-bromophenol (1.5 g, 8.0 mmol) was added and the mixture was heated at 80° C. for 4 hours. After allowing to cool, the reaction mixture was partitioned between ethyl acetate and brine, the organic phase was dried with sodium sulfate, filtered and concentrated. The crude mixture was purified by silica gel column chromatography eluting with a gradient of 20-100% ethyl acetate in hexanes to yield 3-amino-2′,5′-difluoro-[1,1′-biphenyl]-4-ol (1.09 g, 62%).

### Part II—Synthesis of N-(2′,5′-difluoro-4-hydroxy-[1,1′-biphenyl]-3-yl)-3-(trifluoromethyl)benzenesulfonamide

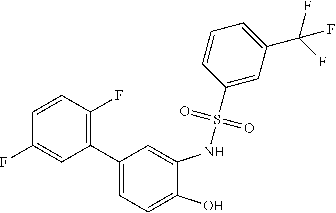 [SMILES: O=S(=O)(Nc1cc(-c2cc(F)ccc2F)ccc1O)c1cccc(C(F)(F)F)c1]

To a solution of 3-amino-2′,5′-difluoro-[1,1′-biphenyl]-4-ol (1.09 g, 4.93 mmol) in anhydrous pyridine (10 mL) under a nitrogen atmosphere was added 3-(trifluoromethyl)benzenesulfonyl chloride (0.82 mL, 5.2 mmol). This mixture was heated at 50° C. overnight. The volume of pyridine was reduced under low pressure then the remaining material was diluted with ethyl acetate (100 mL), washed with 10% citric acid (3×50 mL), then brine, and dried with sodium sulfate, decanted and concentrated onto silica gel. The crude mixture was purified by silica gel column chromatography eluting with a gradient of ethyl acetate in hexanes to yield N-(2′,5′-difluoro-4-hydroxy-[1,1′-biphenyl]-3-yl)-3-(trifluoromethyl)benzenesulfonamide (0.58 g, 27%).

### Part III—Synthesis of 5-(2,5-difluorophenyl)-3-((3-(trifluoromethyl)phenyl)sulfonyl)-2,3-dihydrobenzo[d]oxazole

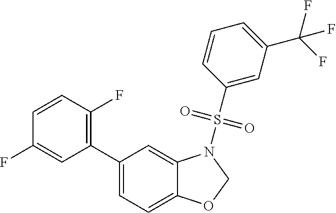 [SMILES: O=S(=O)(c1cccc(C(F)(F)F)c1)N1COc2ccc(-c3cc(F)ccc3F)cc21]

To a solution of N-(2′,5′-difluoro-4-hydroxy-[1,1′-biphenyl]-3-yl)-3-(trifluoromethyl)benzenesulfonamide (35 mg, 0.082 mmol) in dimethylsulfoxide (0.3 mL) was added potassium carbonate (34 mg, 0.24 mmol) followed by diiodomethane (66 mg, 0.24 mmol). This mixture was stirred at 90° C. overnight. The solvent was removed by evaporation and the crude product was purified on a silica gel column eluting with a gradient of 0-40% ethyl acetate in hexanes to yield 5-(2,5-difluorophenyl)-3-((3-(trifluoromethyl)phenyl)sulfonyl)-2,3-dihydrobenzo[d]oxazole (18 mg, 50%). ¹H NMR (400 MHz, DMSO-d₆) δ ppm 8.12 (1H, d, J=7.4 Hz), 8.07 (1H, d, J=8.3 Hz), 7.73-7.85 (2H, m), 7.63-7.72 (1H, m), 7.35-7.46 (2H, m), 7.24-7.35 (2H, m), 6.89 (1H, d, J=8.4 Hz), 5.95 (2H, s). MS m/z 464.19 (M+Na)⁺.

### Example 2—Synthesis of ethyl 2-(6-(2,5-difluorophenyl)-1-((3-(trifluoromethyl)phenyl)sulfonyl)indolin-3-yl)acetate

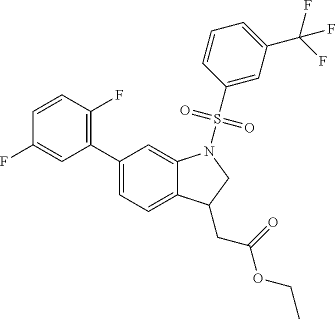 [SMILES: CCOC(=O)CC1CN(S(=O)(=O)c2cccc(C(F)(F)F)c2)c2cc(-c3cc(F)ccc3F)ccc21]

### Part I—Synthesis of ethyl 2-(6-(2,5-difluorophenyl)-1H-indol-3-yl)acetate

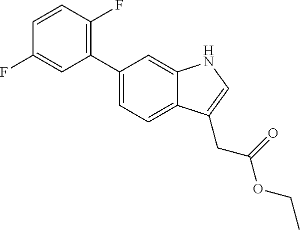 [SMILES: CCOC(=O)Cc1c[nH]c2cc(-c3cc(F)ccc3F)ccc12]

Ethyl 2-(6-bromo-1H-indol-3-yl)acetate (0.30 g, 1.1 mmol), 2,5-difluorophenylboronic acid (0.20 g, 1.3 mmol), and [1,1′-bis(diphenylphosphino)ferrocene]dichloropalladium(II) complex with dichloromethane (0.08 g, 0.11 mmol) were combined in a vial and 1,4-dioxane (3 mL) was added followed by 2M potassium carbonate (1.06 mL, 2.1 mmol). This mixture was heated at 80° C. for 4 hours then cooled, diluted with ethyl acetate, dried with sodium sulfate, filtered and concentrated onto silica gel. The crude mixture was purified by column chromatography eluting with a gradient of ethyl acetate in hexanes to yield ethyl 2-(6-(2,5-difluorophenyl)-1H-indol-3-yl)acetate (230 mg, 69%).

### Part II—Synthesis of ethyl 2-(6-(2,5-difluorophenyl)indolin-3-yl)acetate

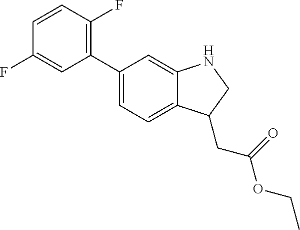 [SMILES: CCOC(=O)CC1CNc2cc(-c3cc(F)ccc3F)ccc21]

To a solution ethyl 2-(6-(2,5-difluorophenyl)-1H-indol-3-yl)acetate (0.1 g, 0.32 mmol) in acetic acid (2 mL) was added sodium cyanoborohydride (60 mg, 0.95 mmol) portionwise. The reaction mixture was stirred at room temperature overnight. Water was then added to the mixture, and the mixture was basified with aqueous sodium hydroxide. The product was extracted into dichloromethane, and the organic extract was dried over sodium sulfate to yield ethyl 2-(6-(2,5-difluorophenyl)indolin-3-yl)acetate (0.1 g).

### Part III—Synthesis of ethyl 2-(6-(2,5-difluorophenyl)-1-((3-(trifluoromethyl)phenyl)sulfonyl)indolin-3-yl)acetate

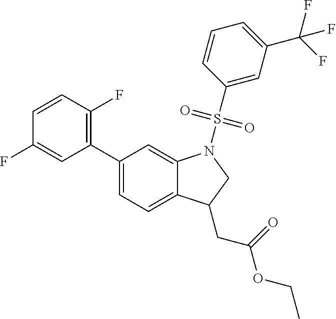 [SMILES: CCOC(=O)CC1CN(S(=O)(=O)c2cccc(C(F)(F)F)c2)c2cc(-c3cc(F)ccc3F)ccc21]

To a solution of ethyl 2-(6-(2,5-difluorophenyl)indolin-3-yl)acetate (0.10 g, 0.32 mmol) in pyridine (2 mL) was added 3-(trifluoromethyl)benzenesulfonyl chloride (0.09 g, 0.38 mmol) and this mixture was heated at 50° C. for 4 hours then concentrated in vacuo. The resulting residue was resuspended in ethyl acetate, washed with 10% citric acid, then brine, and dried with sodium sulfate, filtered and concentrated onto silica gel. The crude mixture was purified by column chromatography eluting with a gradient of ethyl acetate in hexanes to yield ethyl 2-(6-(2,5-difluorophenyl)-1-((3-(trifluoromethyl)phenyl)sulfonyl)indolin-3-yl)acetate (78 mg, 38%). ¹H NMR (400 MHz, DMSO-d₆) δ ppm 8.17 (1H, d, J=7.6 Hz), 8.12 (1H, d, J=8.5 Hz), 8.04 (1H, s), 7.87 (1H, t, J=7.4 Hz), 7.65 (1H, s), 7.58 (1H, s), 7.18-7.46 (4H, m), 4.26 (1H, m), 3.97-4.12 (2H, m), 3.68-3.84 (1H, m), 3.37-3.60 (1H, m), 2.66 (1H, m), 2.32 (1H, m), 1.09-1.23 (3H, m). MS m/z 548.12 (M+Na)⁺.

### Example 3—Synthesis of 2-(6-(2,5-difluorophenyl)-1-((3-(trifluoromethyl)phenyl)sulfonyl)indolin-3-yl)acetic acid

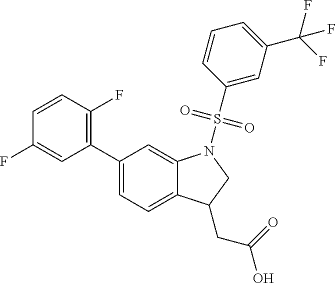 [SMILES: O=C(O)CC1CN(S(=O)(=O)c2cccc(C(F)(F)F)c2)c2cc(-c3cc(F)ccc3F)ccc21]

To a solution of ethyl 2-(6-(2,5-difluorophenyl)-1-((3-(trifluoromethyl)phenyl)sulfonyl)indolin-3-yl)acetate (16 mg, 0.03 mmol) in ethanol (0.4 mL) was added 2M sodium hydroxide (46 μL, 0.09 mmol) and this mixture was stirred at ambient temperature for 4 hours then acidified with 1M hydrogen chloride and extracted with ethyl acetate. The organic extracts were washed with brine, dried with sodium sulfate, filtered and concentrated to provide 2-(6-(2,5-difluorophenyl)-1-((3-(trifluoromethyl)phenyl)sulfonyl)indolin-3-yl)acetic acid (13 mg, 70%). ¹H NMR (400 MHz, DMSO-d₆) δ ppm 12.38 (1H, br s), 8.00-8.21 (3H, m), 7.82-7.94 (1H, m), 7.50-7.68 (1H, m), 7.17-7.46 (5H, m), 4.18-4.31 (1H, m), 3.56-3.81 (1H, m), 3.42-3.55 (1H, m), 2.55-2.72 (1H, m), 2.14-2.36 (1H, m). MS m/z 520.06 (M+Na)⁺.

### Example 4—Synthesis of 5-(2,5-difluorophenyl)-3-((3-(trifluoromethyl)phenyl)sulfonyl)-1H-indole

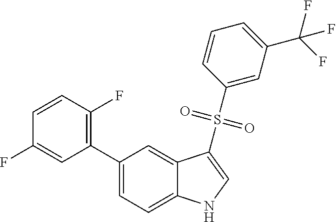 [SMILES: O=S(=O)(c1cccc(C(F)(F)F)c1)c1c[nH]c2ccc(-c3cc(F)ccc3F)cc12]

### Part I—Synthesis of 5-bromo-3-((3-(trifluoromethyl)phenyl)sulfonyl)-1H-indole

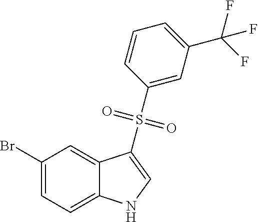 [SMILES: O=S(=O)(c1cccc(C(F)(F)F)c1)c1c[nH]c2ccc(Br)cc12]

5-Bromoindole (0.50 g, 2.6 mmol), [3-(trifluoromethyl)phenyl]sulfinyloxysodium (0.59 g, 2.6 mmol), 1,10-phenanthroline (0.069 g, 0.38 mmol), potassium carbonate (0.71 g, 5.1 mmol) and copper(I) iodide (0.073 g, 0.38 mmol) were combined in anhydrous dimethylsulfoxide (5 mL) in a flask and heated at 70° C. overnight. The resulting solution was cooled, diluted with ethyl acetate, washed with saturated ammonium chloride, then brine, and dried with sodium sulfate, then filtered and concentrated onto silica gel. The crude mixture was purified by column chromatography eluting with a gradient of 0-100% ethyl acetate in hexanes to provide 5-bromo-3-((3-(trifluoromethyl)phenyl)sulfonyl)-1H-indole (0.275 g, 27%).

### Part II—Synthesis of 5-bromo-3-((3-(trifluoromethyl)phenyl)sulfonyl)-1-((2-(trimethylsilyl)ethoxy)methyl)-1H-indole

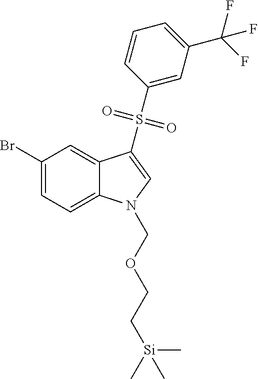 [SMILES: C[Si](C)(C)CCOCn1cc(S(=O)(=O)c2cccc(C(F)(F)F)c2)c2cc(Br)ccc21]

To a solution of 5-bromo-3-((3-(trifluoromethyl)phenyl)sulfonyl)-1H-indole (0.15 g, 0.37 mmol) in anhydrous N,N-dimethylformamide (2 mL) at ambient temperature was added a 60% dispersion of sodium hydride in mineral oil (0.022 g, 0.56 mmol). This mixture was stirred for 5 minutes, then (2-(chloromethoxy)ethyl)trimethylsilane (0.074 g, 0.45 mmol) was added. Stirring was continued at ambient temperature for 4 hours. The reaction was quenched with water and extracted with ethyl acetate. The extracts were washed with brine, dried with sodium sulfate and concentrated onto silica gel. The crude mixture was purified by column chromatography eluting with a gradient of ethyl acetate in hexanes to yield 5-bromo-3-((3-(trifluoromethyl)phenyl)sulfonyl)-1-((2-(trimethylsilyl)ethoxy)methyl)-1H-indole (164 mg, 83%).

### Part III—Synthesis of 5-(2,5-difluorophenyl)-3-((3-(trifluoromethyl)phenyl)sulfonyl)-1-((2-(trimethylsilyl)ethoxy)methyl)-1H-indole

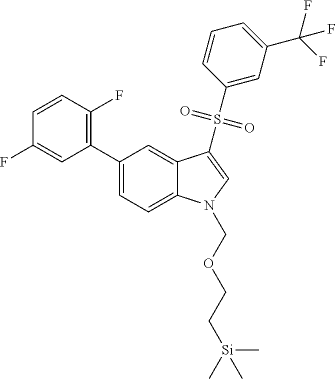 [SMILES: C[Si](C)(C)CCOCn1cc(S(=O)(=O)c2cccc(C(F)(F)F)c2)c2cc(-c3cc(F)ccc3F)ccc21]

To 5-bromo-3-((3-(trifluoromethyl)phenyl)sulfonyl)-1-((2-(trimethylsilyl)ethoxy)methyl)-1H-indole (0.13 g, 0.24 mmol), 2,5-difluorophenylboronic acid (0.058 g, 0.364 mmol), and [1,1′-bis(diphenylphosphino)ferrocene]dichloropalladium(II) complex with dichloromethane (0.019 g, 0.024 mmol) was added 1,4-dioxane (2 mL) and 2M potassium carbonate (0.24 mL, 0.49 mmol). The reaction mixture was heated at 80° C. for 18 hours. The mixture was cooled, then diluted with ethyl acetate and dried with anhydrous sodium sulfate. The solution was decanted and concentrated onto silica gel. The crude product was purified by column chromatography eluting with a gradient of 0-30% ethyl acetate in hexanes to yield 5-(2,5-difluorophenyl)-3-((3-(trifluoromethyl)phenyl)sulfonyl)-1-((2-(trimethylsilyl)ethoxy)methyl)-1H-indole (38 mg, 28%).

### Part IV—Synthesis of 5-(2,5-difluorophenyl)-3-((3-(trifluoromethyl)phenyl)sulfonyl)-1H-indole

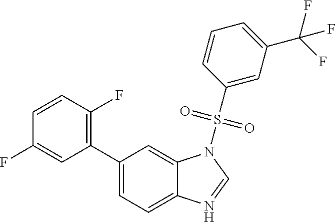 [SMILES: O=S(=O)(c1cccc(C(F)(F)F)c1)n1cnc2ccc(-c3cc(F)ccc3F)cc21]

To a solution of 5-(2,5-difluorophenyl)-3-((3-(trifluoromethyl)phenyl)sulfonyl)-1-((2-(trimethylsilyl)ethoxy)methyl)-1H-indole (14 mg, 0.025 mmol) in anhydrous tetrahydrofuran (1 mL) was added 1M tetrabutylammonium fluoride in tetrahydrofuran (0.12 mL, 0.12 mmol) and the resulting mixture was stirred at ambient temperature overnight. The solvents were removed under reduced pressure and the resulting residue was purified by preparatory HPLC to provide 5-(2,5-difluorophenyl)-3-((3-(trifluoromethyl)phenyl)sulfonyl)-1H-indole (3 mg, 28%). ¹H NMR (400 MHz, CDCl₃-d) 6 ppm 8.87 (1H, br s), 8.33 (1H, s), 8.27 (1H, d, J=8.0 Hz), 8.11 (1H, s), 8.01 (1H, d, J=3.1 Hz), 7.79 (1H, br d, J=7.8 Hz), 7.65 (1H, t, J=7.9 Hz), 7.29-7.57 (2H, m), 7.13-7.24 (2H, m), 7.03 (1H, ddd, J=7.4, 5.3, 3.7 Hz). MS m/z 435.99 (M-H)⁻.

### Example 5—Synthesis of methyl 3-(6-(2,5-difluorophenyl)-1-((3-(trifluoromethyl)phenyl)sulfonyl)indolin-2-yl)propanoate

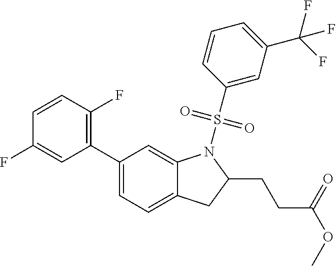 [SMILES: COC(=O)CCC1Cc2ccc(-c3cc(F)ccc3F)cc2N1S(=O)(=O)c1cccc(C(F)(F)F)c1]

### Part I—Synthesis of methyl (E)-3-(6-(2,5-difluorophenyl)-1H-indol-2-yl)acrylate

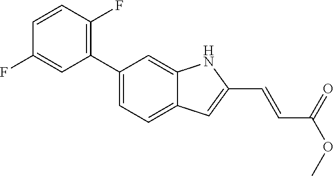 [SMILES: COC(=O)/C=C/c1cc2ccc(-c3cc(F)ccc3F)cc2[nH]1]

Methyl (E)-3-(6-bromo-1H-indol-2-yl)acrylate (1.79 g, 6.4 mmol), 2,5-difluorophenylboronic acid (1.2 g, 7.7 mmol), and 2M potassium carbonate (2.2 g, 17 mmol) were combined in toluene (10 mL), ethanol (2 mL), and water (2 mL). Tetrakis(triphenylphosphine)palladium(0) (0.08 g, 0.11 mmol) was added and the reaction mixture was heated at 100° C. for 18 hours. The crude material was concentrated and purified by column chromatography eluting with a gradient of ethyl acetate in hexanes to yield methyl (E)-3-(6-(2,5-difluorophenyl)-1H-indol-2-yl)acrylate (0.97 g, 48%).

### Part II—Synthesis of methyl 3-(6-(2,5-difluorophenyl)-1H-indol-2-yl)propanoate

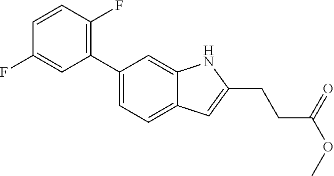 [SMILES: COC(=O)CCc1cc2ccc(-c3cc(F)ccc3F)cc2[nH]1]

To methyl (E)-3-(6-(2,5-difluorophenyl)-1H-indol-2-yl)acrylate (0.97 g, 3.1 mmol) dissolved in methanol (10 mL) was added magnesium metal (0.82 g, 31 mmol). The reaction mixture was stirred at room temperature overnight then concentrated onto silica gel and purified by column chromatography eluting with a gradient of ethyl acetate in hexanes to yield methyl 3-(6-(2,5-difluorophenyl)-1H-indol-2-yl)propanoate (0.66 g, 68%).

### Part III—Synthesis of methyl 3-(6-(2,5-difluorophenyl)indolin-2-yl)propanoate

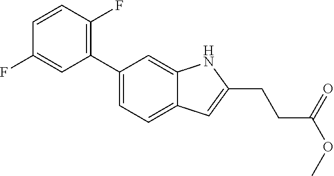 [SMILES: COC(=O)CCc1cc2ccc(-c3cc(F)ccc3F)cc2[nH]1]

To a solution of methyl 3-(6-(2,5-difluorophenyl)-1H-indol-2-yl)propanoate (0.45 g, 1.4 mmol) in acetic acid (4 mL) was added sodium cyanoborohydride (0.27 g, 4.3 mmol) portionwise. The reaction mixture was stirred at room temperature overnight. Water was added, and the mixture was basified with aqueous sodium hydroxide to pH 12. The product was extracted into dichloromethane and the organic extract was dried over sodium sulfate then concentrated onto silica gel and purified by column chromatography eluting with a gradient of ethyl acetate in hexanes to yield methyl 3-(6-(2,5-difluorophenyl)indolin-2-yl)propanoate (0.3 g, 66%).

### Part IV—Synthesis of methyl 3-(6-(2,5-difluorophenyl)-1-((3-(trifluoromethyl)phenyl)sulfonyl)indolin-2-yl)propanoate

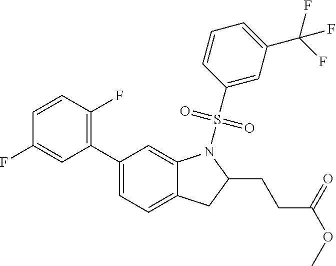 [SMILES: COC(=O)CCC1Cc2ccc(-c3cc(F)ccc3F)cc2N1S(=O)(=O)c1cccc(C(F)(F)F)c1]

To methyl 3-(6-(2,5-difluorophenyl)indolin-2-yl)propanoate (0.3 g, 0.95 mmol) in dichloromethane was added 3-(trifluoromethyl)benzenesulfonyl chloride (0.167 mL, 1.04 mmol) followed by N,N-diisopropylethylamine (0.25 mL, 1.4 mmol) and 4-dimethylaminopyridine (12 mg, 0.1 mmol). The reaction mixture was stirred at room temperature overnight then concentrated onto silica gel and purified by chromatography eluting with a gradient of ethyl acetate in hexanes to yield methyl 3-(6-(2,5-difluorophenyl)-1-((3-(trifluoromethyl)phenyl)sulfonyl)indolin-2-yl)propanoate (0.32 g, 64%). ¹H NMR (400 MHz, DMSO-d₆) δ ppm 7.99-8.12 (2H, m), 7.71-7.86 (2H, m), 7.64 (1H, s), 7.32-7.46 (2H, m), 7.18-7.32 (3H, m), 4.39-4.72 (1H, m), 3.50-3.67 (3H, m), 2.61-2.79 (2H, m), 2.21-2.46 (2H, m), 1.80-1.94 (2H, m). MS m/z 548.18 (M+Na)⁺.

### Example 6—Synthesis of 3-(6-(2,5-difluorophenyl)-1-((3-(trifluoromethyl)phenyl)sulfonyl)indolin-2-yl)propanoic acid

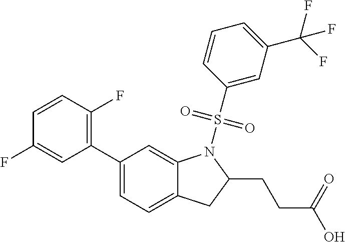 [SMILES: O=C(O)CCC1Cc2ccc(-c3cc(F)ccc3F)cc2N1S(=O)(=O)c1cccc(C(F)(F)F)c1]

Methyl 3-(6-(2,5-difluorophenyl)-1-((3-(trifluoromethyl)phenyl)sulfonyl)indolin-2-yl)propanoate (0.32 g, 0.61 mmol) was dissolved in ethanol and an aqueous solution of sodium hydroxide (0.18 mL, 1.8 mmol) was added. This mixture was stirred at room temperature overnight. When the reaction was complete the mixture was partitioned between ethyl acetate and dilute hydrochloric acid. The organic phase was dried over sodium sulfate then concentrated in vacuo. Purification by chromatography eluting with a gradient of 0-10% methanol in dichloromethane provided 3-(6-(2,5-difluorophenyl)-1-((3-(trifluoromethyl)phenyl)sulfonyl)indolin-2-yl)propanoic acid (0.24 g, 70%). ¹H NMR (400 MHz, DMSO-d₆) δ ppm 7.93-8.08 (2H, m), 7.70-7.89 (2H, m), 7.65 (1H, s), 7.33-7.46 (2H, m), 7.17-7.32 (3H, m), 4.58 (1H, m), 2.56-2.76 (2H, m), 2.29-2.45 (2H, m), 1.71-1.90 (2H, m). MS m/z 534.21 (M+Na)⁺.

### Example 7—Synthesis of methyl 3-(6-(2,5-difluorophenyl)-1-((3-(trifluoromethyl)phenyl)sulfonyl)-1H-indol-2-yl)propanoate

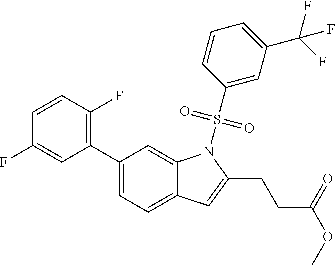 [SMILES: COC(=O)CCc1cc2ccc(-c3cc(F)ccc3F)cc2n1S(=O)(=O)c1cccc(C(F)(F)F)c1]

Methyl 3-(6-(2,5-difluorophenyl)-1H-indol-2-yl)propanoate (90 mg, 0.29 mmol) was dissolved in tetrahydrofuran and the solution was cooled in an ice bath. Sodium hydride (60% dispersion in mineral oil, 13 mg, 0.34 mmol) was added and the mixture was stirred for 30 minutes. 3-(Trifluoromethyl)benzenesulfonyl chloride (84 mg, 0.34 mmol) was added, the reaction mixture was warmed to room temperature, then it was stirred at room temperature for several hours. The reaction was quenched with water, and the pH was adjusted to neutral. The mixture was extracted with ethyl acetate, and the extracts were dried over sodium sulfate, then concentrated onto silica gel. Purification by column chromatography eluting with a gradient of ethyl acetate in hexanes provided methyl 3-(6-(2,5-difluorophenyl)-1-((3-(trifluoromethyl)phenyl)sulfonyl)-1H-indol-2-yl)propanoate (40 mg, 24%). ¹H NMR (400 MHz, CDCl₃-d) 6 ppm 8.64 (1H, m), 8.19 (1H, s), 8.12 (1H, br d, J=7.83 Hz), 7.93 (1H, br d, J=8.61 Hz), 7.73 (1H, br t, J=7.83 Hz), 7.53-7.61 (2H, m), 7.14-7.23 (2H, m), 6.89-6.99 (1H, m), 6.27 (1H, br s), 3.73 (3H, s), 3.04-3.15 (2H, m), 2.75 (2H, t, J=6.65 Hz).

### Example 8—Synthesis of 3-(6-(2,5-difluorophenyl)-1-((3-(trifluoromethyl)phenyl)sulfonyl)-1H-indol-2-yl)propanoic acid

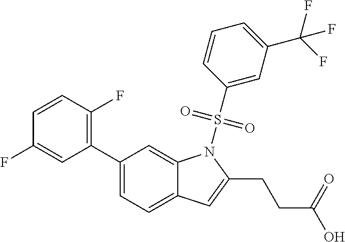 [SMILES: O=C(O)CCc1cc2ccc(-c3cc(F)ccc3F)cc2n1S(=O)(=O)c1cccc(C(F)(F)F)c1]

### Part I—Synthesis of 3-(6-(2,5-difluorophenyl)-1H-indol-2-yl)propanoic acid

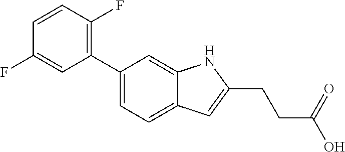 [SMILES: O=C(O)CCc1cc2ccc(-c3cc(F)ccc3F)cc2[nH]1]

To methyl 3-(6-(2,5-difluorophenyl)-1H-indol-2-yl)propanoate (40 mg, 0.089 mmol) in ethanol was added an aqueous solution of sodium hydroxide (9 mg, 0.23 mmol). The reaction mixture was stirred at room temperature for an hour then acidified with aqueous citric acid to pH 2 and extracted with ethyl acetate. The organic solution was dried over sodium sulfate, then concentrated in vacuo. Purification by silica gel column chromatography eluting with a gradient of 0-10% methanol in dichloromethane provided 3-(6-(2,5-difluorophenyl)-1H-indol-2-yl)propanoic acid (17 mg).

### Part II—Synthesis of 3-(6-(2,5-difluorophenyl)-1-((3-(trifluoromethyl)phenyl)sulfonyl)-1H-indol-2-yl)propanoic acid

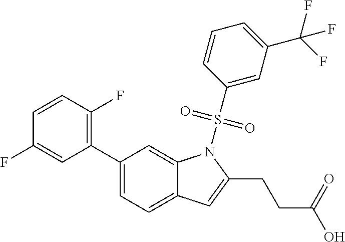 [SMILES: O=C(O)CCc1cc2ccc(-c3cc(F)ccc3F)cc2n1S(=O)(=O)c1cccc(C(F)(F)F)c1]

To 3-(6-(2,5-difluorophenyl)-1H-indol-2-yl)propanoic acid (17 mg, 0.06 mmol) in anhydrous tetrahydrofuran (1 mL) was added a 60% dispersion of sodium hydride in mineral oil (5 mg, 0.12 mmol). This mixture was stirred for 20 minutes at 50° C. then cooled to room temperature. 3-(Trifluoromethyl)benzenesulfonyl chloride (21 mg, 0.09 mmol) was added and the mixture was stirred at room temperature for 3 hours. When the reaction was complete, it was quenched with water, then acidified (pH<4) and extracted with ethyl acetate. The combined extracts were dried with anhydrous sodium sulfate and concentrated. Purification by preparatory HPLC yielded 3-(6-(2,5-difluorophenyl)-1-((3-(trifluoromethyl)phenyl)sulfonyl)-1H-indol-2-yl)propanoic acid (1 mg, 2%). ¹H NMR (400 MHz, DMSO-d₆) δ ppm 12.5 (1H, br s), 8.25 (1H, s), 8.04-8.18 (2H, m), 7.82 (2H, br t, J=8.31 Hz), 7.59 (2H, s), 7.38-7.48 (2H, m), 7.24-7.36 (2H, m), 3.34-3.40 (2H, m), 2.60-2.67 (2H, m). MS m/z 532.01 (M+Na)⁺.

### Example 9—Biological Assays for Agonist Activity Towards RORγ

Exemplary compounds from the above Examples were tested for ability to increase RORγ activity using (i) a RORγ-Ligand Binding Domain (LBD) TR-FRET Assay, and (ii) a Gal4-RORγ Luciferase Reporter Assay in HEK-293T Cells. Assay procedures and results are described below.

### Part I—Procedures for RORγ-Ligand Binding Domain TR-FRET Assay

HIS-tagged RORγ-LBD protein was expressed in SF9 cells using a baculovirus expression system. The lysate was diluted in assay buffer (50 mM Tris pH 7.0, 50 mM KCl, 1 mM EDTA, 0.1 mM DTT, 0.01% BSA) to obtain RORγ-LBD final concentration of −3 nM in a 384-well assay plate (need to titrate for each batch of protein).

A stock of biotinylated-LXXLL peptide from coactivator SRC1 (Biotin-CPSSHSSLTERHKILHRLLQEGSPS) was prepared in assay buffer and added to each well (200 nM final concentration). A solution of Europium tagged anti-HIS antibody (0.6 nM final concentration) and APC-conjugated streptavidin (30 nM final concentration) were also added to each well. RORγ antagonist ursolic acid was also included at a final concentration of 2 μM. Compounds were diluted in DMSO and further diluted in assay buffer with a final DMSO concentration at 1%. The highest concentration of test compound analyzed was 10 μM.

The final assay mixture was incubated overnight at 4° C. or 2 hours at room temperature, and the fluorescence signal was measured on an Envision plate reader: (Excitation filter=340 nm; APC emission=665 nm; Europium emission=615 nm; dichroic mirror=D400/D630; delay time=100 μs, integration time=200 μs). 50% Effective concentration (EC₅₀) values for test compounds were calculated from the quotient of the fluorescence signal at 665 nm divided by the fluorescence signal at 615 nm. The quotient of the fluorescence signals in the absence of ursolic acid or test compound is set as 100. Max Response is defined as the upper plateau in the signal as determined by line-fit using a 4-parameter logistic model in PRISM (GraphPad).

### Part II—Procedures for Gal4-RORγ Luciferase Reporter Assay in HEK-293T Cells

### Transfection of HEK-293 Cells

In the following protocol, HEK-293 cells were transfected with a construct comprising the Gal4 DNA binding domain fused to the ligand binding domain of RORγ (Gal4-RORγ-LBD) in a pcDNA3.1neo plasmid, and also with a reporter construct comprising pGL4.31 Gal4-luciferase (Promega). Control cells were prepared similarly using empty pcDNA3.1neo and pGL4.31 vectors.

Trans-IT reagent (Mirus, 60 μL) at room temperature was added drop wise to OptiMEM (Invitrogen, 1.5 ml). This reagent mixture was mixed by inversion then incubated for 5 to 30 minutes at room temperature. It then was added to a solution of both expression vectors (5 μg each), mixed, and incubated at room temperature for about 20 minutes. HEK-293 cells were harvested from incubation flasks by removing the media, treating with TrypLE Express (Invitrogen), and incubating until the cells detached from the bottom of the flask (approximately 2-5 minutes). 10 Million cells were collected by centrifugation and re-suspended in 10 mL of Dulbecco's Modified Eagle Medium, High Glucose (DMEM, Invitrogen) containing 10% Fetal Bovine Serum and 100 IU each of penicillin and streptomycin. The re-suspended cells and the transfection mixture were added to a T75 flask, mixed and incubated overnight at 37° C. and 5% CO₂.

### Assay for RORγ Activity

The cells were harvested as described above, counted, and centrifuged to obtain the desired number of cells, then re-suspended in complete growth media at 0.75×10⁶ cells/mL. The RORγ antagonist, ursolic acid, was added to the cells at a final concentration of 2 μM. Cells were plated at 20 μL of cell suspension/well (10,000-15,000 cells/well) in white tissue culture treated 384 well plates. Test compounds were dissolved at 10 mM in DMSO then diluted into complete growth medium to 5× the final intended test concentration. These drug stock solutions, 5 μL/well were added to the tissue culture plate. The final DMSO concentration was 0.2%. The plates were briefly centrifuged then incubated overnight at 37° C. and 5% CO₂. To conduct the assay, the tissue culture plates were allowed to equilibrate to room temperature and One-Glo luciferase reagent (Promega, 25 μL/well) was added. The plates were briefly centrifuged then incubated at room temperature for 10 minutes. The luciferase intensity was read on an Envision plate reader (Perkin Elmer). RORγ activity was determined relative to controls and plotted as a function of test compound concentration using PRISM (GraphPad) to determine a 50% effective concentration (EC₅₀). The luciferase signal in the absence of ursolic acid or test compound is defined at 100. The Max Response is the upper plateau in the signal as determined by line-fit using a 4-parameter logistic model in PRISM (GraphPad).

### Part III—Results

Experimental results are provided in Table 10 below. The symbol “++++” indicates an EC₅₀ less than 0.5 μM. The symbol “+++” indicates an EC₅₀ in the range of 0.5 μM to 5 μM. The symbol “++” indicates an EC₅₀ in the range of greater than 5 μM to 10 μM. The symbol “+” indicates an EC₅₀ greater than 10 μM. The symbol “N/A” indicates that no data was available. The symbol “****” indicates a value greater than 200. The symbol “***” indicates a value in the range of greater than 150 to 200. The symbol “**” indicates a value in the range of greater than 90 to 150. The symbol “*” indicates a value in the range of 25 to 90.

TABLE 10

| | TR-FRET Assay | | Gal4-RORγ Assay | |
|---|---|---|---|---|
| Title Compound | | Max | | Max |
| from Example No. | EC₅₀ | Response | EC₅₀ | Response |
| 1 | ++++ | *** | ++++ | ** |
| 2 | ++++ | *** | ++++ | ** |
| 3 | ++++ | ** | ++ | ** |
| 4 | N/A | N/A | + | * |
| 5 | ++++ | *** | ++++ | ** |
| 6 | ++++ | *** | +++ | *** |
| 7 | +++ | *** | +++ | * |
| 8 | +++ | *** | +++ | ** |

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. A compound represented by Formula I: 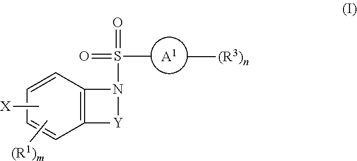
or a pharmaceutically acceptable salt thereof; wherein:
A¹ is phenylene, 5-6 membered heteroarylene, or 3-6 membered heterocycloalkylene;
Y is —C(R^{2B})₂—C(R^{2A})(R^{2B})-ψ, —C(R^{2A})(R^{2B})—C(R^{2B})₂-ψ, —C(R^{2A})═C(R^{2B})-ψ, —C(R^{2B})═C(R^{2A})-ψ, —O—C(R^{2A})(R^{2B})—W, —C(R^{2A})═N-ψ, or —N═C(R^{2A})—; wherein ψ is a bond to the sulfonamide ring nitrogen atom in Formula I;
X is phenyl or 5-10 membered heteroaryl, each of which is optionally substituted by 1, 2, 3, or 4 substituents independently selected from the group consisting of C₁₋₆ alkyl, C₃₋₆ cycloalkyl, halogen, C₁₋₆ haloalkyl, hydroxyl, C₁₋₆ alkoxy, C₁₋₆ haloalkoxy, —O—C₃₋₆ cycloalkyl, —O—(C₁₋₆ alkylene)-OH, cyano, —N(R⁴)(R⁵), —S(O)₂—R⁶, acetyl, and C₆₋₁₀ aryl;
R¹ represents independently for each occurrence halogen, C₁₋₆ alkyl, C₁₋₆ haloalkyl, or C₃₋₆ cycloalkyl;
R^{2A} is —(C₁₋₆ alkylene)-A², —(C₃₋₆ cycloalkylene)-A², -(2-6 membered heteroalkylene)-A², —(C₁₋₃ alkylene)-(C₃₋₆ cycloalkylene)-(C₀₋₃ alkylene)-A², —(C₁₋₃ alkylene)-(3-6 membered heterocycloalkylene)-(C₀₋₃ alkylene)-A², —(C₆₋₁₀ arylene)-A², C₂₋₆ hydroxyalkyl, —CO₂R⁴, or hydrogen; wherein:
A² is —CO₂R⁴, —C(O)-A³, —C(O)R⁶, —C(O)N(R⁴)(R⁵), —C(O)N(R⁴)—(C₁₋₄ alkylene)-CO₂R⁴, —C(O)N(R⁴)SO₂R⁴, —C(O)N(R⁴)SO₂A³, —C(O)N(R⁴)—(C₁₋₆ alkylene)-N(R⁷)C(O)R⁶, —C(O)N(R⁴)—(C₁₋₆ alkylene)-SO₂N(R⁷)₂, —C(O)N(R⁴)—(C₁₋₆ alkylene)-CN, —C(O)N(R⁴)—(C₁₋₆ alkylene)-OC(O)R⁶, —N(R⁴)C(O)R⁷, —N(R⁴)C(O)A³, —N(R⁴)C(O)—(C₁₋₆ alkylene)-CO₂R⁴, —N(R⁴)C(O)—(C₁₋₆ alkylene)-N(R⁷)C(O)R⁶, —N(R⁴)C(O)—(C₁₋₆ alkylene)-SO₂N(R⁷)₂, —N(R⁴)C(O)—(C₁₋₆ alkylene)-CN, —N(R⁴)C(O)—(C₁₋₆ alkylene)-OC(O)R⁶, —N(R⁴)C(O)N(R⁴)—(C₁₋₆ alkylene)-CO₂R⁴, —N(R⁴)C(O)N(R⁷)₂, —N(R⁴)CO₂R⁶, —N(R⁴)S(O)₂R⁷, —N(R⁴)S(O)₂N(R⁴)(R⁵), —N(R⁴)(R⁵), hydroxyl, or A³;
A³ is aryl, C₃₋₆ cycloalkyl, or a 5-8 membered heterocyclic group, each of which is optionally substituted with 1, 2, or 3 substituents independently selected from the group consisting of C₁₋₆ alkyl, C₁₋₆ haloalkyl, C₃₋₆ cycloalkyl, halogen, C₁₋₆ alkoxy, C₁₋₆ haloalkoxy, oxo, —C(O)R⁶, —CO₂R⁷, —C(O)N(R⁴)(R⁵), —N(R⁴)C(O)(R⁶), and —N(R⁴)(R⁵); and
any alkylene, cycloalkylene, or heteroalkylene within the definition of R^{2A} is optionally substituted by 1, 2, or 3 substitutents independently selected from the group consisting of hydroxyl and C₁₋₆ alkoxy;
R^{2B} represents independently for each occurrence hydrogen, C₁₋₆ alkyl, or C₁₋₃ haloalkyl;
R³ represents independently for each occurrence hydrogen, C₁₋₆ haloalkyl, halogen, hydroxyl, C₁₋₆ alkyl, C₃₋₆ cycloalkyl, C₁₋₆ alkoxy, C₁₋₆ haloalkoxy, —O—(C₁₋₆ alkylene)-OH, or —O—(C₁₋₆ alkylene)-CO₂R⁴; or two vicinal occurrences of R³ are taken together with intervening atoms to form a 4-6 membered ring optionally substituted with 1, 2, or 3 substituents independently selected from the group consisting of C₁₋₆ alkyl, C₁₋₆ haloalkyl, C₃₋₆ cycloalkyl, halogen, C₁₋₆ alkoxy, C₁₋₆ haloalkoxy, —C(O)R⁶, and —CO₂R⁷;
R⁴ and R⁵ each represent independently for each occurrence hydrogen, C₁₋₆ alkyl, C₁₋₆ haloalkyl, C₁₋₆ hydroxyalkyl, or C₃₋₆ cycloalkyl; or an occurrence of R⁴ and R⁵ attached to the same nitrogen atom are taken together with the nitrogen atom to which they are attached to form a 3-7 membered heterocyclic ring;
R⁶ represents independently for each occurrence C₁₋₆ alkyl, C₃₋₆ cycloalkyl, or aralkyl;
R⁷ represents independently for each occurrence hydrogen, C₁₋₆ alkyl, C₁₋₆ hydroxyalkyl, C₁₋₆ haloalkyl, C₁₋₆ hydroxyhaloalkyl, C₃₋₆ cycloalkyl, C₃₋₆ hydroxycycloalkyl, —(C₁₋₆ alkylene)-(C₃₋₆ cycloalkyl), —(C₁₋₆ alkylene)-(C₂₋₄ alkenyl), or aralkyl;
m is 0, 1, or 2; and
n is 1, 2, or 3.

2. The compound of claim 1, wherein A¹ is phenylene or 5-6 membered heteroarylene.

3. The compound of claim 1, wherein Y is —C(R^{2B})₂—C(R^{2A})(R^{2B})—ψ or —C(R^{2A})(R^{2B})—C(R^{2B})₂-ψ.

4. (canceled)

5. The compound of claim 1, wherein the compound is represented by Formula I-A or I-A′: 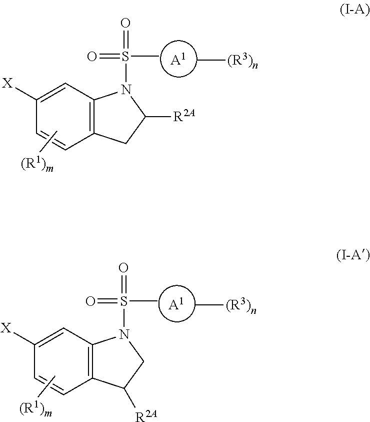
or a pharmaceutically acceptable salt thereof; wherein:
A¹ is phenylene or a 5-6 membered heteroarylene;
X is phenyl or 5-6 membered heteroaryl, each of which is optionally substituted by 1, 2, or 3 substituents independently selected from the group consisting of C₁₋₆ alkyl, C₃₋₆ cycloalkyl, halogen, C₁₋₆ haloalkyl, hydroxyl, C₁₋₆ alkoxy, C₁₋₆ haloalkoxy, —O—C₃₋₆ cycloalkyl, —O—(C₁₋₆ alkylene)-OH, cyano, —N(R⁴)(R⁵), and C₆₋₁₀ aryl;
R¹ represents independently for each occurrence halogen, C₁₋₆ alkyl, or C₁₋₆ haloalkyl;
R^{2A} is —(C₁₋₆ alkylene)-A², —(C₃₋₆ cycloalkylene)-A², -(2-6 membered heteroalkylene)-A², —(C₁₋₃ alkylene)-(C₃₋₆ cycloalkylene)-(C₀₋₃ alkylene)-A², —(C₁₋₃ alkylene)-(3-6 membered heterocycloalkylene)-(C₀₋₃ alkylene)-A², or —CO₂R⁴; wherein A² is —CO₂R⁴, —C(O)-A³, —C(O)N(R⁴)(R⁵), —C(O)N(R⁴)—(C₁₋₄ alkylene)-CO₂R⁴, —N(R⁴)C(O)R⁷, —N(R⁴)C(O)-A³, —N(R⁴)C(O)—(C₁₋₆ alkylene)-CO₂R⁴, —N(R⁴)C(O)N(R⁴)—(C₁₋₆ alkylene)-CO₂R⁴, —N(R⁴)CO₂R⁶, or A³; and A³ is a 5-8 membered heterocyclic group optionally substituted with 1, 2, or 3 substituents independently selected from the group consisting of C₁₋₆ alkyl, C₁₋₆ haloalkyl, C₃₋₆ cycloalkyl, halogen, C₁₋₆ alkoxy, C₁₋₆ haloalkoxy, oxo, —C(O)R⁶, —CO₂R⁷, and —C(O)N(R⁴)(R⁵);
R³ represents independently for each occurrence hydrogen, C₁₋₆ haloalkyl, halogen, hydroxyl, C₁₋₆ alkyl, C₃₋₆ cycloalkyl, C₁₋₆ alkoxy, C₁₋₆ haloalkoxy, —O—(C₁₋₆ alkylene)-OH, or —O—(C₁₋₆ alkylene)-CO₂R⁴;
R⁴ and R⁵ each represent independently for each occurrence hydrogen, C₁₋₆ alkyl, or C₃₋₆ cycloalkyl; or an occurrence of R⁴ and R⁵ attached to the same nitrogen atom are taken together with the nitrogen atom to which they are attached to form a 3-7 membered heterocyclic ring;
R⁶ represents independently for each occurrence C₁₋₆ alkyl, C₃₋₆ cycloalkyl, or aralkyl;
R⁷ represents independently for each occurrence hydrogen, C₁₋₆ alkyl, C₁₋₆ hydroxyalkyl, C₃₋₆ cycloalkyl, C₃₋₆ hydroxycycloalkyl, or aralkyl;
m is 0, 1, or 2; and
n is 1, 2, or 3.

6. (canceled)

7. The compound of claim 1, wherein the compound is represented by Formula I-B or I-B′: 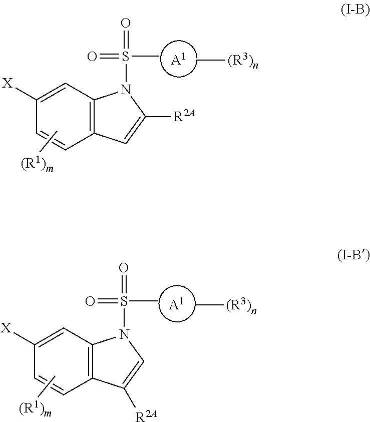
or a pharmaceutically acceptable salt thereof; wherein:
A¹ is phenylene or a 5-6 membered heteroarylene;
X is phenyl or 5-6 membered heteroaryl, each of which is optionally substituted by 1, 2, or 3 substituents independently selected from the group consisting of C₁₋₆ alkyl, C₃₋₆ cycloalkyl, halogen, C₁₋₆ haloalkyl, hydroxyl, C₁₋₆ alkoxy, C₁₋₆ haloalkoxy, —O—C₃₋₆ cycloalkyl, —O—(C₁₋₆ alkylene)-OH, cyano, —N(R⁴)(R⁵), and C₆₋₁₀ aryl;
R¹ represents independently for each occurrence halogen, C₁₋₆ alkyl, or C₁₋₆ haloalkyl;
R^{2A} is —(C₁₋₆ alkylene)-A², —(C₃₋₆ cycloalkylene)-A², -(2-6 membered heteroalkylene)-A², —(C₁₋₃ alkylene)-(C₃₋₆ cycloalkylene)-(C₀₋₃ alkylene)-A², —(C₁₋₃ alkylene)-(3-6 membered heterocycloalkylene)-(C₀₋₃ alkylene)-A², or —CO₂R⁴; wherein A² is —CO₂R⁴, —C(O)-A³, —C(O)N(R⁴)(R⁵), —C(O)N(R⁴)—(C₁₋₄ alkylene)-CO₂R⁴, —N(R⁴)C(O)R⁷, —N(R⁴)C(O)-A³, —N(R⁴)C(O)—(C₁₋₆ alkylene)-CO₂R⁴, —N(R⁴)C(O)N(R⁴)—(C₁₋₆ alkylene)-CO₂R⁴, —N(R⁴)CO₂R⁶, or A³; and A³ is a 5-8 membered heterocyclic group optionally substituted with 1, 2, or 3 substituents independently selected from the group consisting of C₁₋₆ alkyl, C₁₋₆ haloalkyl, C₃₋₆ cycloalkyl, halogen, C₁₋₆ alkoxy, C₁₋₆ haloalkoxy, oxo, —C(O)R⁶, —CO₂R⁷, and —C(O)N(R⁴)(R⁵);
R³ represents independently for each occurrence hydrogen, C₁₋₆ haloalkyl, halogen, hydroxyl, C₁₋₆ alkyl, C₃₋₆ cycloalkyl, C₁₋₆ alkoxy, C₁₋₆ haloalkoxy, —O—(C₁₋₆ alkylene)-OH, or —O—(C₁₋₆ alkylene)-CO₂R⁴;
R⁴ and R⁵ each represent independently for each occurrence hydrogen, C₁₋₆ alkyl, or C₃₋₆ cycloalkyl; or an occurrence of R⁴ and R⁵ attached to the same nitrogen atom are taken together with the nitrogen atom to which they are attached to form a 3-7 membered heterocyclic ring;
R⁶ represents independently for each occurrence C₁₋₆ alkyl, C₃₋₆ cycloalkyl, or aralkyl;
R⁷ represents independently for each occurrence hydrogen, C₁₋₆ alkyl, C₁₋₆ hydroxyalkyl, C₃₋₆ cycloalkyl, C₃₋₆ hydroxycycloalkyl, or aralkyl;
m is 0, 1, or 2; and
n is 1, 2, or 3.

8. (canceled)

9. The compound of claim 1, wherein the compound is represented by Formula I-C: 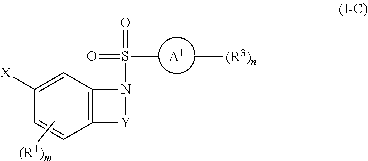
or a pharmaceutically acceptable salt thereof; wherein:
A¹ is phenylene or a 5-6 membered heteroarylene;
Y is —O—C(R^{2A})(R^{2B})-ψ, —C(R^{2A})═N-ψ, or —N═C(R^{2A})-ψ; wherein ψ is a bond to the sulfonamide ring nitrogen atom in Formula I-C;
X is phenyl or 5-6 membered heteroaryl, each of which is optionally substituted by 1, 2, or 3 substituents independently selected from the group consisting of C₁₋₆ alkyl, C₃₋₆ cycloalkyl, halogen, C₁₋₆ haloalkyl, hydroxyl, C₁₋₆ alkoxy, C₁₋₆ haloalkoxy, —O—C₃₋₆ cycloalkyl, —O—(C₁₋₆ alkylene)-OH, cyano, —N(R⁴)(R⁵), and C₆₋₁₀ aryl;
R¹ represents independently for each occurrence halogen, C₁₋₆ alkyl, or C₁₋₆ haloalkyl;
R^{2A} is —(C₁₋₆ alkylene)-A², —(C₃₋₆ cycloalkylene)-A², -(2-6 membered heteroalkylene)-A², —(C₁₋₃ alkylene)-(C₃₋₆ cycloalkylene)-(C₀₋₃ alkylene)-A², —(C₁₋₃ alkylene)-(3-6 membered heterocycloalkylene)-(C₀₋₃ alkylene)-A², or —CO₂R⁴; wherein A² is —CO₂R⁴, —C(O)-A³, —C(O)N(R⁴)(R⁵), —C(O)N(R⁴)—(C₁₋₄ alkylene)-CO₂R⁴, —N(R⁴)C(O)R⁷, —N(R⁴)C(O)-A³, —N(R⁴)C(O)—(C₁₋₆ alkylene)-CO₂R⁴, —N(R⁴)C(O)N(R⁴)—(C₁₋₆ alkylene)-CO₂R⁴, —N(R⁴)CO₂R⁶, or A³; and A³ is a 5-8 membered heterocyclic group optionally substituted with 1, 2, or 3 substituents independently selected from the group consisting of C₁₋₆ alkyl, C₁₋₆ haloalkyl, C₃₋₆ cycloalkyl, halogen, C₁₋₆ alkoxy, C₁₋₆ haloalkoxy, oxo, —C(O)R⁶, —CO₂R⁷, and —C(O)N(R⁴)(R⁵);
R^{2B} is hydrogen, C₁₋₆ alkyl, or C₁₋₃ haloalkyl;
R³ represents independently for each occurrence hydrogen, C₁₋₆ haloalkyl, halogen, hydroxyl, C₁₋₆ alkyl, C₃₋₆ cycloalkyl, C₁₋₆ alkoxy, C₁₋₆ haloalkoxy, —O—(C₁₋₆ alkylene)-OH, or —O—(C₁₋₆ alkylene)-CO₂R⁴;
R⁴ and R⁵ each represent independently for each occurrence hydrogen, C₁₋₆ alkyl, or C₃₋₆ cycloalkyl; or an occurrence of R⁴ and R⁵ attached to the same nitrogen atom are taken together with the nitrogen atom to which they are attached to form a 3-7 membered heterocyclic ring;
R⁶ represents independently for each occurrence C₁₋₆ alkyl, C₃₋₆ cycloalkyl, or aralkyl;
R⁷ represents independently for each occurrence hydrogen, C₁₋₆ alkyl, C₁₋₆ hydroxyalkyl, C₃₋₆ cycloalkyl, C₃₋₆ hydroxycycloalkyl, or aralkyl;
m is 0, 1, or 2; and
n is 1, 2, or 3.

10-17. (canceled)

18. The compound of claim 1, wherein R^{2A} is —(C₁₋₆ alkylene)-A².

19. The compound of claim 9, wherein R^{2A} is —(C₂₋₃ alkylene)-A².

20-21. (canceled)

22. The compound of claim 19, wherein A² is —CO₂R⁴.

23-36. (canceled)

37. The compound of claim 1, wherein X is phenyl substituted by 1, 2, or 3 substituents independently selected from the group consisting of halogen, C₁₋₃ haloalkyl, C₁₋₃ alkoxy, and C₁₋₃ haloalkoxy.

38-42. (canceled)

43. The compound of claim 1, wherein the compound is represented by Formula I-D: 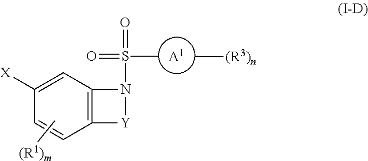
or a pharmaceutically acceptable salt thereof; wherein:
A¹ is phenylene or a 5-6 membered heteroarylene;
Y is —O—C(R^{2A})(R^{2B})-ψ; wherein ψ is a bond to the sulfonamide ring nitrogen atom in Formula I-D;
X is:
(i) phenyl substituted by 1, 2, or 3 substituents independently selected from the group consisting of C₁₋₆ alkyl, C₃₋₆ cycloalkyl, halogen, C₁₋₆ haloalkyl, hydroxyl, C₁₋₆ alkoxy, C₁₋₆ haloalkoxy, —O—C₃₋₆ cycloalkyl, —O—(C₁₋₆ alkylene)-OH, cyano, —N(R⁴)(R⁵), and C₆₋₁₀ aryl; or
(ii) 5-6 membered heteroaryl optionally substituted by 1, 2, or 3 substituents independently selected from the group consisting of C₁₋₆ alkyl, C₃₋₆ cycloalkyl, halogen, C₁₋₆ haloalkyl, hydroxyl, C₁₋₆ alkoxy, C₁₋₆ haloalkoxy, —O—C₃₋₆ cycloalkyl, —O—(C₁₋₆ alkylene)-OH, cyano, —N(R⁴)(R⁵), and C₆₋₁₀ aryl;
R¹ represents independently for each occurrence halogen, C₁₋₆ alkyl, or C₁₋₆ haloalkyl;
R^{2A} is hydrogen;
R^{2B} is hydrogen, C₁₋₆ alkyl, or C₁₋₃ haloalkyl;
R³ represents independently for each occurrence C₁₋₆ haloalkyl, halogen, hydroxyl, C₁₋₆ alkyl, C₃₋₆ cycloalkyl, C₁₋₆ alkoxy, C₁₋₆ haloalkoxy, —O—(C₁₋₆ alkylene)-OH, or —O—(C₁₋₆ alkylene)-CO₂R⁴;
R⁴ and R⁵ each represent independently for each occurrence hydrogen, C₁₋₆ alkyl, or C₃₋₆ cycloalkyl; or an occurrence of R⁴ and R⁵ attached to the same nitrogen atom are taken together with the nitrogen atom to which they are attached to form a 3-7 membered heterocyclic ring;
m is 0, 1, or 2; and
n is 1, 2, or 3.

44. (canceled)

45. (canceled)

46. A compound represented by Formula II: 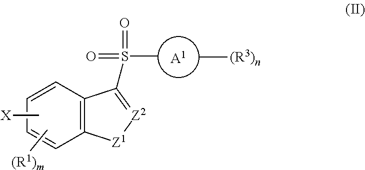
or a pharmaceutically acceptable salt thereof; wherein:
A¹ is phenylene, 5-6 membered heteroarylene, or 3-6 membered heterocycloalkylene;
Z¹ and Z² are defined by:
(i) Z¹ is O or —N(R^{2B})—; and Z² is C(R^{2A}); or
(ii) Z¹ is —N(R^{2A})—; and Z² is N;
X is phenyl or 5-10 membered heteroaryl, each of which is optionally substituted by 1, 2, 3, or 4 substituents independently selected from the group consisting of C₁₋₆ alkyl, C₃₋₆ cycloalkyl, halogen, C₁₋₆ haloalkyl, hydroxyl, C₁₋₆ alkoxy, C₁₋₆ haloalkoxy, —O—C₃₋₆ cycloalkyl, —O—(C₁₋₆ alkylene)-OH, cyano, —N(R⁴)(R⁵), —S(O)₂—R⁶, acetyl, and C₆₋₁₀ aryl;
R¹ represents independently for each occurrence halogen, C₁₋₆ alkyl, C₁₋₆ haloalkyl, or C₃₋₆ cycloalkyl;
R^{2A} is —(C₁₋₆ alkylene)-A², —(C₃₋₆ cycloalkylene)-A², -(2-6 membered heteroalkylene)-A², —(C₁₋₃ alkylene)-(C₃₋₆ cycloalkylene)-(C₀₋₃ alkylene)-A², —(C₁₋₃ alkylene)-(3-6 membered heterocycloalkylene)-(C₀₋₃ alkylene)-A², —(C₆₋₁₀ arylene)-A², C₂₋₆ hydroxyalkyl, —CO₂R⁴, or hydrogen; wherein
A² is —CO₂R⁴, —C(O)-A³, —C(O)R⁶, —C(O)N(R⁴)(R⁵), —C(O)N(R⁴)—(C₁₋₄ alkylene)-CO₂R⁴, —C(O)N(R⁴)SO₂R⁴, —C(O)N(R⁴)SO₂A³, —C(O)N(R⁴)—(C₁₋₆ alkylene)-N(R⁷)C(O)R⁶, —C(O)N(R⁴)—(C₁₋₆ alkylene)-SO₂N(R)₂, —C(O)N(R⁴)—(C₁₋₆ alkylene)-CN, —C(O)N(R⁴)—(C₁₋₆ alkylene)-OC(O)R⁶, —N(R⁴)C(O)R⁷, —N(R⁴)C(O)A³, —N(R⁴)C(O)—(C₁₋₆ alkylene)-CO₂R⁴, —N(R⁴)C(O)—(C₁₋₆ alkylene)-N(R⁷)C(O)R⁶, —N(R⁴)C(O)—(C₁₋₆ alkylene)-SO₂N(R⁷)₂, —N(R⁴)C(O)—(C₁₋₆ alkylene)-CN, —N(R⁴)C(O)—(C₁₋₆ alkylene)-OC(O)R⁶, —N(R⁴)C(O)N(R⁴)—(C₁₋₆ alkylene)-CO₂R⁴, —N(R⁴)C(O)N(R⁷)₂, —N(R⁴)CO₂R⁶, —N(R⁴)S(O)₂R⁷, —N(R⁴)S(O)₂N(R⁴)(R⁵), —N(R⁴)(R⁵), hydroxyl, or A³;
A³ is aryl, C₃₋₆ cycloalkyl, or a 5-8 membered heterocyclic group, each of which is optionally substituted with 1, 2, or 3 substituents independently selected from the group consisting of C₁₋₆ alkyl, C₁₋₆ haloalkyl, C₃₋₆ cycloalkyl, halogen, C₁₋₆ alkoxy, C₁₋₆ haloalkoxy, oxo, —C(O)R⁶, —CO₂R⁷, —C(O)N(R⁴)(R⁵), —N(R⁴)C(O)(R⁶), and —N(R⁴)(R⁵); and
any alkylene, cycloalkylene, or heteroalkylene within the definition of R^{2A} is optionally substituted by 1, 2, or 3 substitutents independently selected from the group consisting of hydroxyl and C₁₋₆ alkoxy;
R^{2B} represents independently for each occurrence hydrogen, C₁₋₆ alkyl, or C₁₋₃ haloalkyl;
R³ represents independently for each occurrence hydrogen, C₁₋₆ haloalkyl, halogen, hydroxyl, C₁₋₆ alkyl, C₃₋₆ cycloalkyl, C₁₋₆ alkoxy, C₁₋₆ haloalkoxy, —O—(C₁₋₆ alkylene)-OH, or —O—(C₁₋₆ alkylene)-CO₂R⁴; or two vicinal occurrences of R³ are taken together with intervening atoms to form a 4-6 membered ring optionally substituted with 1, 2, or 3 substituents independently selected from the group consisting of C₁₋₆ alkyl, C₁₋₆ haloalkyl, C₃₋₆ cycloalkyl, halogen, C₁₋₆ alkoxy, C₁₋₆ haloalkoxy, —C(O)R⁶, and —CO₂R⁷;
R⁴ and R⁵ each represent independently for each occurrence hydrogen, C₁₋₆ alkyl, C₁₋₆ haloalkyl, C₁₋₆ hydroxyalkyl, or C₃₋₆ cycloalkyl; or an occurrence of R⁴ and R⁵ attached to the same nitrogen atom are taken together with the nitrogen atom to which they are attached to form a 3-7 membered heterocyclic ring;
R⁶ represents independently for each occurrence C₁₋₆ alkyl, C₃₋₆ cycloalkyl, or aralkyl;
R⁷ represents independently for each occurrence hydrogen, C₁₋₆ alkyl, C₁₋₆ hydroxyalkyl, C₁₋₆ haloalkyl, C₁₋₆ hydroxyhaloalkyl, C₃₋₆ cycloalkyl, C₃₋₆ hydroxycycloalkyl, —(C₁₋₆ alkylene)-(C₃₋₆ cycloalkyl), —(C₁₋₆ alkylene)-(C₂₋₄ alkenyl), or aralkyl;
m is 0, 1, or 2; and
n is 1, 2, or 3.

47-85. (canceled)

86. A compound in Table 10 herein or a pharmaceutically acceptable salt thereof.

87. A pharmaceutical composition comprising a compound of claim 1 and a pharmaceutically acceptable carrier.

88. A method of treating a disorder selected from the group consisting of cancer, bacterial infection, fungal infection, and immune deficiency disorder, comprising administering a therapeutically effective amount of a compound of claim 1 to a subject in need thereof to treat the disorder.

89. The method of claim 88, wherein the disorder is cancer.

90. The method of claim 88, wherein the disorder is colon cancer, pancreatic cancer, breast cancer, ovarian cancer, prostate cancer, squamous cell carcinoma, basal cell carcinoma, adenocarcinoma, sweat gland carcinoma, sebaceous gland carcinoma, lung cancer, leukemia, bladder cancer, stomach cancer, cervical cancer, testicular cancer, skin cancer, rectal cancer, thyroid cancer, kidney cancer, uterus cancer, espophagus cancer, liver cancer, an acoustic neuroma, oligodendroglioma, meningioma, neuroblastoma, or retinoblastoma.

91. A method of increasing the amount of IL-17 in a subject, comprising administering to a subject an effective amount of a compound of claim 1 to increase the amount of IL-17 in the subject.

92. The method of claim 88, wherein the subject is a human.

93. A method of promoting the activity of RORγ, comprising exposing a RORγ to an effective amount of a compound of claim 1 to promote the activity of said RORγ.
